(19) United States
(12) Patent Application Publication
(10) Pub. No.: US 2005/0070712 A1
(43) Pub. Date: Mar. 31, 2005

(54) Pyrimidine derivatives as ghrelin receptor modulators

(76) Inventors: Christi Kosogof, Crystal Lake, IL (US); Bo Liu, Libertyville, IL (US); Gang Liu, Gurnee, IL (US); Mei Liu, Gurnee, IL (US); Lissa T.J. Nelson, Highland Park, IL (US); Michael D. Serby, Mundelein, IL (US); Hing L. Sham, Vernon Hills, IL (US); Bruce G. Szczepankiewicz, Lindenhurst, IL (US); Zhili Xin, Lake Bluff, IL (US); Hongyu Zhao, Buffalo Grove, IL (US)

Correspondence Address: ROBERT DEBERARDINE;ABBOTT LABORATORIES, 100 ABBOTT PARK ROAD, DEPT. 377/AP6A, ABBOTT PARK, IL, 60064-6008, US

(21) Appl. No.: 10/671,723

(22) Filed: Sep. 26, 2003

(51) Int. Cl. A61K 31/513; C07D 43/04

(52) U.S. Cl. 544/310; 544/333; 514/256; 514/269

(57) ABSTRACT

The present invention is related to compounds of formula (I), 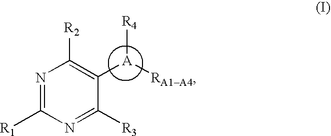 or a therapeutically suitable salt or prodrug thereof, the preparation of the compounds, compositions containing the compounds and the use of the compounds in the prevention or treatment of disorders regulated by ghrelin including anorexia, cancer cachexia, eating disorders, age-related decline in body composition, weight gain, obesity, and diabetes mellitus.

### TECHNICAL FIELD

The present invention is directed to compounds that are modulators of the ghrelin receptor, the preparation of the compounds, compositions containing the compounds and the use of the compounds in the prevention or treatment of disorders regulated by ghrelin including anorexia, cancer cachexia, eating disorders, age-related decline in body composition, weight gain, obesity, and diabetes mellitus.

### BACKGROUND OF THE INVENTION

Stimulation of food intake is important in connection with patients suffering from anorexia due to chronic medical conditions, eating disorders, age-related decline in body composition, and other conditions in which excessive weight loss has produced a detrimental effect on the patients' health.

Obesity is a common and very serious public health problem as it increases a person's risk for a number of serious conditions, including diabetes, heart disease, stroke, high blood pressure, and some types of cancers. Considerable increase in the number of obese individuals over the past two decades has created profound public health implications. Although studies have demonstrated that reduction in obesity by diet and exercise reduces the associated risk factors dramatically, these treatments are largely unsuccessful considering obesity is strongly associated with genetically inherited factors that contribute to increased appetite, preferences for highly caloric foods, reduced physical activity, and increased lipogenic metabolism.

Growth hormone (GH) is not only of importance for linear body growth but is also of major importance for the maintenance of body composition, metabolism and heart function in adult life. GH release from the anterior pituitary is regulated by the stimulatory peptide GH-releasing hormone (GHRH) and the inhibitory peptide somatostatin, Frohman, L., Jansson, J.-O., Endocr. Rev. (1986) 7:223-253. Early research identified small GH-releasing peptides (GHRPs) derived from the pentapeptide met-enkephalin, Momany, F., et. al., Endocrinology (1981) 108:31-39. Further efforts led to the development of a number of peptidyl and non-peptidyl growth hormone secrectgogues (GHSs), including the orally-active, non-peptidyl GH secretagogue MK677, Svensson, J., et. al., J. Clin. Endocrinol. Metab. (1998) 83:362-369. Later efforts cloned a seven-transmembrane G-protein coupled receptor (GPCR) that was a target for the GHSs, Howard, A., et. al., Science (1996) 273:974-977.

This GHS-receptor (GHS-R) is localized in the hypothalamus and in the pituitary, but also in other brain areas such as the hippocampus as well as the pancreas. Recently, an endogenous ligand for the GHS-R, ghrelin, an acylated peptide consisting of 28 amino acids was isolated, Kojima, M., et. al., Nature (1999) 402:656-660. Since then, ghrelin has been found to be localized in the hypothalamic-pituitary area where it stimulates the release of GH to the circulation, but is also found in the highest concentration in the stomach.

Biological evidence indicates that ghrelin has an important role in the regulation of metabolism and energy expenditure. Ghrelin was found to stimulate food intake and weight gain when administered either systemically or intraventricularly in rodents, Nakazato M, et. al., Nature 2001 ;409: 194-198; Asakawa A, et. al., Gastroenterology (2001) 120:337-345. Ghrelin was also found to be more potent than any other orexigenic peptide except neuropeptide Y (NPY). The orexigenic activity of centrally administered ghrelin is thought to be mediated by brain NPY and AGRP, two neuropeptides with potent orexigenic actions, Kamegai, J., et. al., Endocrinology (2000) 141:4797-4800. It was also recognized that the appetite activity of centrally administered ghrelin can be blocked by co-administration of a NPY-Y1 receptor antagonist. In addition, ghrelin was found to reverse leptin-induced inhibition of food intake, Shintani, M., et. al., Diabetes (2001) 50:227-232. Ghrelin exerts its actions in the arcuate nucleus and paraventricular nucleus to influence the interplay of NPY, AGRP and a-MSH circuits. Ghrelin may also act via afferent vagal pathways that terminate in the hypothalamus. In obese patients, the increase in the plasma ghrelin level with diet-induced weight loss is consistent with the hypothesis that ghrelin has a role in the long-term regulation of body weight. Gastric bypass in obese patients is associated with markedly suppressed ghrelin levels, possibly contributing to the weight-reducing effect of the procedure, Cummings, D. E., et. al., N Engl J Med (2002) 346:1623-30.

Intracerebroventricular treatment with the anti-ghrelin antiserum against the N-terminal region twice a day for 5 days in rats decreased significantly both daily food intake and body weight, Murakami, N., et. al., Journal of Endocrinology (2002) 174, 283-288. Transgenic (Tg) rats expressing an antisense ghrelin receptor mRNA under the control of the promoter for tyrosine hydroxylase (TH) selectively attenuated ghrelin receptor protein expression in the arcuate nucleus (Arc). Tg rats had lower body weight and less adipose tissue than did control rats. Daily food intake was reduced, and the stimulatory effect of GHS treatment on feeding was abolished in Tg rats, Shuto, Y., et. al., J. Clin. Invest. (2002) 109:1429-1436. More recently, a peptide-based GHS-R antagonist, [D-Lys-3]-GHRP, was found to decrease energy intake in lean mice, in mice with diet induced obesity, and in ob/ob obese mice. It also reduced the rate of gastric emptying. Repeated aministration of this GHS-R antagonist decreased body weight and improved glycemic control in ob/ob mice, Asakawa, A. et. al., Gut, (2003), 52:947-952. These data suggest that ghrelin receptor modulators may be beneficial in the treatment of anorexia, cancer cachexia, eating disorders, age-related decline in body composition, weight gain, obesity and disorders associated with obesity such as diabetes mellitus.

### SUMMARY OF THE INVENTION

The principle embodiment of the present invention is directed to a compound of formula (I), 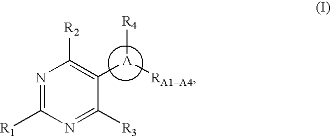
or a therapeutically suitable salt or prodrug thereof, wherein
R₁ is a member selected from the group consisting of hydrogen, alkyl, alkoxy, aryl, arylalkyl, cyano, cycloalkyl, cycloalkylalkyl, haloalkoxy, haloalkyl, halogen, heteroaryl, heteroarylalkyl, heterocycle, heterocyclealkyl, hydroxy, mercapto, nitro, and —NR_{A}R_{B};
R_{A} and R_{B} are each independently a member selected from the group consisting of hydrogen, alkoxycarbonyl, alkyl, alkylcarbonyl, alkoxysulfonyl, alkylsulfonyl, aryl, arylalkyl, and formyl;
R₂ is a member selected from the group consisting of hydrogen, alkyl, alkoxy, alkoxycarbonyl, aryl, arylalkyl, cyano, cycloalkyl, cycloalkylalkyl, haloalkoxy, haloalkyl, halogen, heteroaryl, heteroarylalkyl, heterocycle, heterocyclealkyl, hydroxy, mercapto, nitro, —NR_{C}R_{D}, and (NR_{C}R_{D})alkyl;
R_{C} and R_{D} are each independently a member selected from the group consisting of hydrogen, alkoxycarbonyl, alkenyl, alkyl, alkylcarbonyl, alkoxysulfonyl, alkylsulfonyl, aryl, arylalkyl, cycloalkyl, cycloalkylalkyl, formyl, and hydroxyalkyl;
R₃ is a member selected from the group consisting of alkenyl, alkenylalkoxyalkyl, alkenyloxy, alkenyloxyalkyl, alkoxy, alkoxyalkoxy, alkoxyalkoxyalkyl, alkoxyalkyl, alkoxycarbony, alkoxycarbonylalkyl, alkoxysulfonyl, alkylcarbonyl, alkylcarbonylalkyl, alkylcarbonyloxy, alkylsulfinyl, alkylsulfinylalkyl, alkylsulfonyl, alkylsulfonylalkyl, alkylthio, alkylthioalkyl, alkynyl, alkynylalkoxyalkyl, alkynyloxy, alkynyloxyalkyl, aryl, arylalkoxy, arylalkoxyalkyl, arylalkyl, arylalkylthio, arylalkylthioalkyl, aryloxy, aryloxyalkyl, arylthio, arylthioalkyl, carboxy, carboxyalkyl, cyanoalkyl, cycloalkenyl, cycloalkenylalkoxy, cycloalkenylalkoxyalkyl, cycloalkenylalkyl, cycloalkenylalkylthio, cycloalkenylalkylthioalkyl, cycloalkenyloxy, cycloalkenyloxyalkyl, cycloalkenylthio, cycloalkenylthioalkyl, cycloalkyl, cycloalkylalkoxy, cycloalkylalkoxyalkyl, cycloalkylalkyl, cycloalkylalkylthio, cycloalkylalkylthioalkyl, cycloalkyloxy, cycloalkyloxyalkyl, cycloalkylthio, cycloalkylthioalkyl, formyl, haloalkoxy, halogen, heteroaryl, heteroarylalkoxy, heteroarylalkoxyalkyl, heteroarylalkyl, heteroarylalkylthio, heteroarylalkylthioalkyl, heteroaryloxy, heteroaryloxyalkyl, heteroarylthio, heteroarylthioalkyl, heterocycle, heterocyclealkoxy, heterocyclealkoxyalkyl, heterocyclealkyl, heterocyclealkylthio, heterocyclealkylthioalkyl, heterocycleoxy, heterocycleoxyalkyl, heterocyclethio, heterocyclethioalkyl, hydroxy, hydroxyalkyl, mercapto, nitro, —NR_{E}R_{H}, (NR_{E}R_{H})alkyl, (NR_{E}R_{F})carbonylalkenyl, (NR_{E}R_{F})carbonylalkyl, (NR_{E}R_{F})sulfonyl, and (NR_{E}R_{F})sulfonylalkyl;
R_{E} and R_{F} are each independently a member selected from the group consisting of hydrogen, alkoxyalkyl, alkoxyalkylcarbonyl, alkoxycarbonyl, alkoxysulfonyl, alkyl, alkylcarbonyl, alkylsulfonyl, alkylthioalkyl, alkylthioalkylcarbonyl, alkylthiocarbonyl, aryl, arylalkoxyalkyl, arylalkyl, arylcarbonyl, cycloalkyl, cycloalkylalkyl, cycloalkylcarbonyl, formyl, heteroaryl, heteroarylalkyl, heteroarylcarbonyl, heterocycle, heterocyclealkyl, heterocyclecarbonyl, (NZ₁Z₂)alkyl, and (NZ₁Z₂)carbonyl;
Z₁ and Z₂ are each independently a member selected from the group consisting of hydrogen, alkoxycarbonyl, alkyl, alkylcarbonyl, alkoxysulfonyl, alkylsulfonyl, aryl, arylalkyl, arylcarbonyl, cycloalkyl, cycloalkylalkyl, cycloalkylcarbonyl, formyl, heteroaryl, heteroarylalkyl, heteroarylcarbonyl, heterocycle, heterocyclealkyl, and heterocyclecarbonyl;
R₄ is a member selected from the group consisting of alkenyl, alkenyloxy, alkenyloxyalkyl, alkoxy, alkoxyalkoxy, alkoxyalkoxyalkyl, alkoxyalkyl, alkoxycarbonyl, alkoxycarbonylalkyl, alkoxysulfonyl, alkyl, alkylcarbonyl, alkylcarbonylalkyl, alkylcarbonyloxy, alkylsulfinyl, alkylsulfinylalkyl, alkylsulfonyl, alkylsulfonylalkyl, alkylthio, alkylthioalkyl, alkynyl, alkynyloxy, alkynyloxyalkyl, aryl, arylalkoxy, arylalkoxyalkyl, arylalkyl, arylalkylthio, arylalkylthioalkyl, aryloxy, aryloxyalkyl, arylthio, arylthioalkyl, carboxy, carboxyalkyl, cyano, cyanoalkyl, cycloalkenyl, cycloalkenylalkoxy, cycloalkenylalkoxyalkyl, cycloalkenylalkyl, cycloalkenylalkylthio, cycloalkenylalkylthioalkyl, cycloalkenyloxy, cycloalkenyloxyalkyl, cycloalkenylthio, cycloalkenylthioalkyl, cycloalkyl, cycloalkylalkoxy, cycloalkylalkoxyalkyl, cycloalkylalkyl, cycloalkylalkylthio, cycloalkylalkylthioalkyl, cycloalkyloxy, cycloalkyloxyalkyl, cycloalkylthio, cycloalkylthioalkyl, formyl, formylalkyl, haloalkoxy, haloalkyl, halogen, heteroaryl, heteroarylalkoxy, heteroarylalkoxyalkyl, heteroarylalkyl, heteroarylalkylthio, heteroarylalkylthioalkyl, heteroaryloxy, heteroaryloxyalkyl, heteroarylthio, heteroarylthioalkyl, heterocycle, heterocyclealkoxy, heterocyclealkoxyalkyl, heterocyclealkyl, heterocyclealkylthio, heterocyclealkylthioalkyl, heterocycleoxy, heterocycleoxyalkyl, heterocyclethio, heterocyclethioalkyl, hydroxy, hydroxyalkyl, mercapto, nitro, —NR_{G}R_{H}, (NR_{G}R_{H})alkyl, (NR_{G}R_{H})carbonyl, and (NR_{G}R_{H})sulfonyl;
R_{G} and R_{H} are each independently a member selected from the group consisting of hydrogen, alkoxyalkyl, alkoxyalkylcarbonyl, alkoxycarbonyl, alkoxysulfonyl, alkyl, alkylcarbonyl, alkylsulfonyl, alkylthioalkyl, alkylthioalkylcarbonyl, alkylthiocarbonyl, aryl, arylalkoxyalkyl, arylalkyl, arylcarbonyl, cycloalkyl, cycloalkylalkoxyalkyl, cycloalkylalkyl, cycloalkylcarbonyl, formyl, heteroaryl, heteroarylalkoxyalkyl, heteroarylalkyl, heteroarylcarbonyl, heterocycle, heterocyclealkoxyalkyl, heterocyclealkyl, heterocyclecarbonyl, (NZ₃Z₄)alkyl, and (NZ₃Z₄)carbonyl;
Z₃ and Z₄ are each independently a member selected from the group consisting of hydrogen, alkoxycarbonyl, alkoxysulfonyl, alkyl, alkylcarbonyl, alkylsulfonyl, aryl, arylalkyl, arylcarbonyl, cycloalkyl, cycloalkylalkyl, cycloalkylcarbonyl, formyl, heteroaryl, heteroarylalkyl, heteroarylcarbonyl, heterocycle, heterocyclealkyl, and heterocyclecarbonyl;
A is a member selected from the group consisting of aryl, cycloalkyl, cycloalkenyl, heteroaryl, and heterocycle;
R_{A1}, R_{A2}, R_{A3}, and R_{A4} are each independently a member selected from the group consisting of hydrogen, alkenyl, alkenyloxy, alkoxy, alkoxyalkoxy, alkoxyalkoxyalkyl, alkoxyalkyl, alkoxycarbonyl, alkoxycarbonylalkyl, alkoxysulfonyl, alkyl, alkylcarbonyl, alkylcarbonylalkyl, alkylcarbonyloxy, alkylsulfinyl, alkylsulfinylalkyl, alkylsulfonyl, alkylsulfonylalkyl, alkylthio, alkylthioalkyl, alkynyl, aryl, carboxy, carboxyalkyl, cyano, cyanoalkyl, cycloalkyl, formyl, haloalkoxy, haloalkyl, halogen, heteroaryl, heterocycle,hydroxy, hydroxyalkyl, mercapto, nitro, —NR_{J}R_{K}, (NR_{J}R_{K})alkyl, (NR_{J}R_{K})carbonyl, and (NR_{J}R_{K})sulfonyl; and
R_{J} and R_{K} are each independently a member selected from the group consisting of hydrogen, alkoxycarbonyl, alkyl, alkylcarbonyl, alkoxysulfonyl, alkylsulfonyl, aryl, arylalkyl, and formyl.

According to another embodiment, the present invention is directed to a pharmaceutical composition comprising a therapeutically effective amount of a compound of formula (I) and a pharmaceutically suitable carrier.

According to another embodiment, the present invention is directed to a method of treating a disorder regulated by ghrelin receptors in a mammal, comprising administring of a compound of formula (I).

According to another embodiment, the present invention is directed to a method of treating anorexia, cancer cachexia, eating disorders, age-related decline in body composition, weight gain, obesity, or diabetes mellitus in a mammal comprising administring a compound of formula (I).

### DETAILED DESCRIPTION OF THE INVENTION

According to an embodiment of the present invention, there is disclosed a compound of formula (I), or a therapeutically suitable salt or prodrug thereof, wherein R₁ is —NR_{A}R_{B}; R₂ is a member selected from the group consisting of —NR_{C}R_{D} and (NR_{C}R_{D})alkyl; R_{A} and R_{B} are hydrogen; R_{C}, and R_{D} are each independently a member selected from the group consisting of hydrogen, alkyl, aryl, and arylalkyl; R₃ is a member selected from the group consisting of alkoxyalkoxyalkyl, alkoxyalkyl, arylalkoxyalkyl, arylalkyl, aryloxyalkyl, cycloalkylalkoxyalkyl, heterocycle, heterocyclealkoxyalkyl, and (NR_{E}R_{F})carbonylalkyl; R_{E} and R_{F} are each independently a member selected from the group consisting of hydrogen, alkyl, aryl, and arylalkyl; R₄ is a member selected from the group consisting of arylalkoxy, heteroarylalkoxy, —NR_{G}R_{H}, (NR_{G}R_{H})alkyl; R_{G} is a member selected from the group consisting of hydrogen, alkyl, and alkylcarbonyl; R_{H} is a member selected from the group consisting of alkoxyalkylcarbonyl, alkyl, alkylcarbonyl, aryl, arylalkoxyalkyl, arylalkyl, arylcarbonyl, cycloalkyl, cycloalkylalkyl, heteroaryl, heteroarylalkyl, heterocyclealkyl, (NZ₃Z₄)alkyl, and (NZ₃Z₄)carbonyl; Z₃ and Z₄ are each independently a member selected from the group consisting of hydrogen and, alkoxycarbonyl; A is aryl; and R_{A1}, R_{A2}, R_{A3}, and R_{A4} are each independently a member selected from the group consisting of hydrogen and halogen.

According to another embodiment, the present invention is directed to a compound of formula (I), or a therapeutically suitable salt or prodrug thereof, wherein R₁ is —NR_{A}R_{B}; R₂ is a member selected from the group consisting of —NR_{C}R_{D} and (NR_{C}R_{D})alkyl; R_{A}, R_{B}, R_{C}, and R_{D} are each hydrogen; R₃ is a member selected from the group consisting of alkoxyalkoxyalkyl, alkoxyalkyl, arylalkoxyalkyl, arylalkyl, aryloxyalkyl, cycloalkylalkoxyalkyl, heterocycle, heterocyclealkoxyalkyl, and (NR_{E}R_{F})carbonylalkyl; R_{E} and R_{F} are each independently a member selected from the group consisting of hydrogen, alkyl, aryl, and arylalkyl; R₄ is a member selected from the group consisting of arylalkoxy, heteroarylalkoxy, —NR_{G}R_{H}, (NR_{G}R_{H})alkyl; R_{G} is a member selected from the group consisting of hydrogen, alkyl, and alkylcarbonyl; R_{H} is a member selected from the group consisting of alkoxyalkylcarbonyl, alkyl, alkylcarbonyl, aryl, arylalkoxyalkyl, arylalkyl, arylcarbonyl, cycloalkyl, cycloalkylalkyl, heteroaryl, heteroarylalkyl, heterocyclealkyl, (NZ₃Z₄)alkyl, and (NZ₃Z₄)carbonyl; Z₃ and Z₄ are each independently a member selected from the group consisting of hydrogen and alkoxycarbonyl; A is aryl; and R_{A1}, R_{A2}, R_{A3}, and R_{A4} are each hydrogen and halogen.

According to another embodiment of the present invention, there is disclosed a compound of formula (Ia), 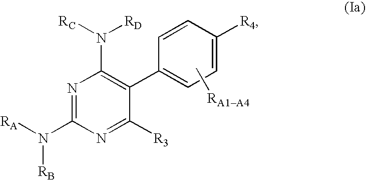
or a therapeutically suitable salt or prodrug thereof, wherein
R_{A} and R_{B} are each independently a member selected from the group consisting of hydrogen, alkoxycarbonyl, alkyl, alkylcarbonyl, alkoxysulfonyl, alkylsulfonyl, aryl, arylalkyl, and formyl;
R_{C} and R_{D} are each independently a member selected from the group consisting of hydrogen, alkoxycarbonyl, alkenyl, alkyl, alkylcarbonyl, alkoxysulfonyl, alkylsulfonyl, aryl, arylalkyl, cycloalkyl, cycloalkylalkyl, formyl, and hydroxyalkyl;
R₃ is a member selected from the group consisting of alkenyl, alkenylalkoxyalkyl, alkenyloxy, alkenyloxyalkyl, alkoxy, alkoxyalkoxy, alkoxyalkoxyalkyl, alkoxyalkyl, alkoxycarbony, alkoxycarbonylalkyl, alkoxysulfonyl, alkylcarbonyl, alkylcarbonylalkyl, alkylcarbonyloxy, alkylsulfinyl, alkylsulfinylalkyl, alkylsulfonyl, alkylsulfonylalkyl, alkylthio, alkylthioalkyl, alkynyl, alkynylalkoxyalkyl, alkynyloxy, alkynyloxyalkyl, aryl, arylalkoxy, arylalkoxyalkyl, arylalkyl, arylalkylthio, arylalkylthioalkyl, aryloxy, aryloxyalkyl, arylthio, arylthioalkyl, carboxy, carboxyalkyl, cyanoalkyl, cycloalkenyl, cycloalkenylalkoxy, cycloalkenylalkoxyalkyl, cycloalkenylalkyl, cycloalkenylalkylthio, cycloalkenylalkylthioalkyl, cycloalkenyloxy, cycloalkenyloxyalkyl, cycloalkenylthio, cycloalkenylthioalkyl, cycloalkyl, cycloalkylalkoxy, cycloalkylalkoxyalkyl, cycloalkylalkyl, cycloalkylalkylthio, cycloalkylalkylthioalkyl, cycloalkyloxy, cycloalkyloxyalkyl, cycloalkylthio, cycloalkylthioalkyl, formyl, haloalkoxy, halogen, heteroaryl, heteroarylalkoxy, heteroarylalkoxyalkyl, heteroarylalkyl, heteroarylalkylthio, heteroarylalkylthioalkyl, heteroaryloxy, heteroaryloxyalkyl, heteroarylthio, heteroarylthioalkyl, heterocycle, heterocyclealkoxy, heterocyclealkoxyalkyl, heterocyclealkyl, heterocyclealkylthio, heterocyclealkylthioalkyl, heterocycleoxy, heterocycleoxyalkyl, heterocyclethio, heterocyclethioalkyl, hydroxy, hydroxyalkyl, mercapto, nitro, —NR_{E}R_{H}, (NR_{E}R_{H})alkyl, (NR_{E}R_{F})carbonylalkenyl, (NR_{E}R_{F})carbonylalkyl, (NR_{E}R_{F})sulfonyl, and (NR_{E}R_{F})sulfonylalkyl;
R_{E} and R_{F} are each independently a member selected from the group consisting of hydrogen, alkoxyalkyl, alkoxyalkylcarbonyl, alkoxycarbonyl, alkoxysulfonyl, alkyl, alkylcarbonyl, alkylsulfonyl, alkylthioalkyl, alkylthioalkylcarbonyl, alkylthiocarbonyl, aryl, arylalkoxyalkyl, arylalkyl, arylcarbonyl, cycloalkyl, cycloalkylalkyl, cycloalkylcarbonyl, formyl, heteroaryl, heteroarylalkyl, heteroarylcarbonyl, heterocycle, heterocyclealkyl, heterocyclecarbonyl, (NZ₁Z₂)alkyl, and (NZ₁Z₂)carbonyl;
Z₁ and Z₂ are each independently a member selected from the group consisting of hydrogen, alkoxycarbonyl, alkyl, alkylcarbonyl, alkoxysulfonyl, alkylsulfonyl, aryl, arylalkyl, arylcarbonyl, cycloalkyl, cycloalkylalkyl, cycloalkylcarbonyl, formyl, heteroaryl, heteroarylalkyl, heteroarylcarbonyl, heterocycle, heterocyclealkyl, and heterocyclecarbonyl;
R₄ is a member selected from the group consisting of alkenyl, alkenyloxy, alkenyloxyalkyl, alkoxy, alkoxyalkoxy, alkoxyalkoxyalkyl, alkoxyalkyl, alkoxycarbonyl, alkoxycarbonylalkyl, alkoxysulfonyl, alkyl, alkylcarbonyl, alkylcarbonylalkyl, alkylcarbonyloxy, alkylsulfinyl, alkylsulfinylalkyl, alkylsulfonyl, alkylsulfonylalkyl, alkylthio, alkylthioalkyl, alkynyl, alkynyloxy, alkynyloxyalkyl, aryl, arylalkoxy, arylalkoxyalkyl, arylalkyl, arylalkylthio, arylalkylthioalkyl, aryloxy, aryloxyalkyl, arylthio, arylthioalkyl, carboxy, carboxyalkyl, cyano, cyanoalkyl, cycloalkenyl, cycloalkenylalkoxy, cycloalkenylalkoxyalkyl, cycloalkenylalkyl, cycloalkenylalkylthio, cycloalkenylalkylthioalkyl, cycloalkenyloxy, cycloalkenyloxyalkyl, cycloalkenylthio, cycloalkenylthioalkyl, cycloalkyl, cycloalkylalkoxy, cycloalkylalkoxyalkyl, cycloalkylalkyl, cycloalkylalkylthio, cycloalkylalkylthioalkyl, cycloalkyloxy, cycloalkyloxyalkyl, cycloalkylthio, cycloalkylthioalkyl, formyl, formylalkyl, haloalkoxy, haloalkyl, halogen, heteroaryl, heteroarylalkoxy, heteroarylalkoxyalkyl, heteroarylalkyl, heteroarylalkylthio, heteroarylalkylthioalkyl, heteroaryloxy, heteroaryloxyalkyl, heteroarylthio, heteroarylthioalkyl, heterocycle, heterocyclealkoxy, heterocyclealkoxyalkyl, heterocyclealkyl, heterocyclealkylthio, heterocyclealkylthioalkyl, heterocycleoxy, heterocycleoxyalkyl, heterocyclethio, heterocyclethioalkyl, hydroxy, hydroxyalkyl, mercapto, nitro, —NR_{G}R_{H}, (NR_{G}R_{H})alkyl, (NR_{G}R_{H})carbonyl, and (NR_{G}R_{H})sulfonyl;
R_{G} and R_{H} are each independently a member selected from the group consisting of hydrogen, alkoxyalkyl, alkoxyalkylcarbonyl, alkoxycarbonyl, alkoxysulfonyl, alkyl, alkylcarbonyl, alkylsulfonyl, alkylthioalkyl, alkylthioalkylcarbonyl, alkylthiocarbonyl, aryl, arylalkoxyalkyl, arylalkyl, arylcarbonyl, cycloalkyl, cycloalkylalkoxyalkyl, cycloalkylalkyl, cycloalkylcarbonyl, formyl, heteroaryl, heteroarylalkoxyalkyl, heteroarylalkyl, heteroarylcarbonyl, heterocycle, heterocyclealkoxyalkyl, heterocyclealkyl, heterocyclecarbonyl, (NZ₃Z₄)alkyl, and (NZ₃Z₄)carbonyl;
Z₃ and Z₄ are each independently a member selected from the group consisting of hydrogen, alkoxycarbonyl, alkoxysulfonyl, alkyl, alkylcarbonyl, alkylsulfonyl, aryl, arylalkyl, arylcarbonyl, cycloalkyl, cycloalkylalkyl, cycloalkylcarbonyl, formyl, heteroaryl, heteroarylalkyl, heteroarylcarbonyl, heterocycle, heterocyclealkyl, and heterocyclecarbonyl;
R_{A1}, R_{A2}, R_{A3}, and R_{A4} are each independently a member selected from the group consisting of hydrogen, alkenyl, alkenyloxy, alkoxy, alkoxyalkoxy, alkoxyalkoxyalkyl, alkoxyalkyl, alkoxycarbonyl, alkoxycarbonylalkyl, alkoxysulfonyl, alkyl, alkylcarbonyl, alkylcarbonylalkyl, alkylcarbonyloxy, alkylsulfinyl, alkylsulfinylalkyl, alkylsulfonyl, alkylsulfonylalkyl, alkylthio, alkylthioalkyl, alkynyl, aryl, carboxy, carboxyalkyl, cyano, cyanoalkyl, cycloalkyl, formyl, haloalkoxy, haloalkyl, halogen, heteroaryl, heterocyle, hydroxy, hydroxyalkyl, mercapto, nitro, —NR_{J}R_{K}, (NR_{J}R_{K})alkyl, (NR_{J}R_{K})carbonyl, and (NR_{J}R_{K})sulfonyl; and
R_{J} and R_{K} are each independently a member selected from the group consisting of hydrogen, alkoxycarbonyl, alkyl, alkylcarbonyl, alkoxysulfonyl, alkylsulfonyl, aryl, arylalkyl, and formyl.

According to another embodiment, the present invention is directed to a compound of formula (Ia) wherein R_{A} and R_{B} are each hydrogen; R_{C} and R_{D} are each independently a member selected from the group consisting of hydrogen, alkyl, aryl, and arylalkyl; R₃ is a member selected from the group consisting of alkoxyalkoxyalkyl, alkoxyalkyl, arylalkoxyalkyl, arylalkyl, aryloxyalkyl, cycloalkylalkoxyalkyl, heterocycle, heterocyclealkoxyalkyl, and (NR_{E}R_{F})carbonylalkyl; R_{E} and R_{F} are each independently a member selected from the group consisting of hydrogen, alkyl, aryl, and arylalkyl; R₄ is a member selected from the group consisting of arylalkoxy, heteroarylalkoxy, —NR_{G}R_{H}, (NR_{G}R_{H})alkyl; R_{G} is a member selected from the group consisting of hydrogen, alkyl, and alkylcarbonyl; R_{H} is a member selected from the group consisting of alkoxyalkylcarbonyl, alkyl, alkylcarbonyl, aryl, arylalkoxyalkyl, arylalkyl, arylcarbonyl, cycloalkyl, cycloalkylalkyl, heteroaryl, heteroarylalkyl, heterocyclealkyl, (NZ₃Z₄)alkyl, and (NZ₃Z₄)carbonyl; Z₃ and Z₄ are each independently a member selected from the group consisting of hydrogen and alkoxycarbonyl; and R_{A1}, R_{A2}, R_{A3,} and R_{A4} are each independently a member selected from the group consisting of hydrogen and halogen.

According to another embodiment, the present invention is directed to a compound of formula (Ia) wherein R_{A}, R_{B}, R_{C}, and R_{D} are each hydrogen; R₃ is a member selected from the group consisting of alkoxyalkoxyalkyl, alkoxyalkyl, arylalkoxyalkyl, arylalkyl, aryloxyalkyl, cycloalkylalkoxyalkyl, heterocycle, heterocyclealkoxyalkyl, and (NR_{E}R_{F})carbonylalkyl; R_{E} and R_{F} are each independently a member selected from the group consisting of hydrogen, alkyl, aryl, and arylalkyl; R₄ is a member selected from the group consisting of arylalkoxy, heteroarylalkoxy, —NR_{G}R_{H}, (NR_{G}R_{H})alkyl; R_{G} is a member selected from the group consisting of hydrogen, alkyl, and alkylcarbonyl; R_{H} is a member selected from the group consisting of alkoxyalkylcarbonyl, alkyl, alkylcarbonyl, aryl, arylalkoxyalkyl, arylalkyl, arylcarbonyl, cycloalkyl, cycloalkylalkyl, heteroaryl, heteroarylalkyl, heterocyclealkyl, (NZ₃Z₄)alkyl, and (NZ₃Z₄)carbonyl; Z₃ and Z₄ are each independently a member selected from the group consisting of hydrogen and alkoxycarbonyl; and R_{A1}, R_{A2}, R_{A3}, and R_{A4} are each independently a member selected from the group consisting of hydrogen and halogen.

According to another embodiment, the present invention is directed to a pharmaceutical composition comprising a therapeutically effective amount of a compound of formula (Ia) in combination with a pharmaceutically suitable carrier.

According to another embodiment, the present invention is directed to a method of treating a disorder regulated by ghrelin receptors in a mammal, comprising administration of a compound of formula (Ia).

According to another embodiment, the present invention is directed to a method of treating anorexia, cancer cachexia, eating disorders, age-related decline in body composition, weight gain, obesity, or diabetes mellitus in a mammal comprising administration of a compound of formula (Ia).

According to another embodiment of the present invention, there is disclosed a compound of formula (Ib), 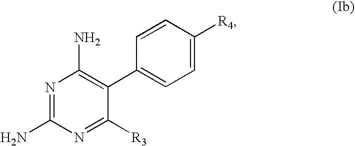
or a therapeutically suitable salt or prodrug thereof, wherein
R₃ is a member selected from the group consisting of alkenyl, alkenylalkoxyalkyl, alkenyloxy, alkenyloxyalkyl, alkoxy, alkoxyalkoxy, alkoxyalkoxyalkyl, alkoxyalkyl, alkoxycarbony, alkoxycarbonylalkyl, alkoxysulfonyl, alkylcarbonyl, alkylcarbonylalkyl, alkylcarbonyloxy, alkylsulfinyl, alkylsulfinylalkyl, alkylsulfonyl, alkylsulfonylalkyl, alkylthio, alkylthioalkyl, alkynyl, alkynylalkoxyalkyl, alkynyloxy, alkynyloxyalkyl, aryl, arylalkoxy, arylalkoxyalkyl, arylalkyl, arylalkylthio, arylalkylthioalkyl, aryloxy, aryloxyalkyl, arylthio, arylthioalkyl, carboxy, carboxyalkyl, cyanoalkyl, cycloalkenyl, cycloalkenylalkoxy, cycloalkenylalkoxyalkyl, cycloalkenylalkyl, cycloalkenylalkylthio, cycloalkenylalkylthioalkyl, cycloalkenyloxy, cycloalkenyloxyalkyl, cycloalkenylthio, cycloalkenylthioalkyl, cycloalkyl, cycloalkylalkoxy, cycloalkylalkoxyalkyl, cycloalkylalkyl, cycloalkylalkylthio, cycloalkylalkylthioalkyl, cycloalkyloxy, cycloalkyloxyalkyl, cycloalkylthio, cycloalkylthioalkyl, formyl, haloalkoxy, halogen, heteroaryl, heteroarylalkoxy, heteroarylalkoxyalkyl, heteroarylalkyl, heteroarylalkylthio, heteroarylalkylthioalkyl, heteroaryloxy, heteroaryloxyalkyl, heteroarylthio, heteroarylthioalkyl, heterocycle, heterocyclealkoxy, heterocyclealkoxyalkyl, heterocyclealkyl, heterocyclealkylthio, heterocyclealkylthioalkyl, heterocycleoxy, heterocycleoxyalkyl, heterocyclethio, heterocyclethioalkyl, hydroxy, hydroxyalkyl, mercapto, nitro, —NR_{E}R_{H}, (NR_{E}R_{H})alkyl, (NR_{E}R_{F})carbonylalkenyl, (NR_{E}R_{F})carbonylalkyl, (NR_{E}R_{F})sulfonyl, and (NR_{E}R_{F})sulfonylalkyl;
R_{E} and R_{F} are each independently a member selected from the group consisting of hydrogen, alkoxyalkyl, alkoxyalkylcarbonyl, alkoxycarbonyl, alkoxysulfonyl, alkyl, alkylcarbonyl, alkylsulfonyl, alkylthioalkyl, alkylthioalkylcarbonyl, alkylthiocarbonyl, aryl, arylalkoxyalkyl, arylalkyl, arylcarbonyl, cycloalkyl, cycloalkylalkyl, cycloalkylcarbonyl, formyl, heteroaryl, heteroarylalkyl, heteroarylcarbonyl, heterocycle, heterocyclealkyl, heterocyclecarbonyl, (NZ₁Z₂)alkyl, and (NZ₁Z₂)carbonyl;
Z₁ and Z₂ are each independently a member selected from the group consisting of hydrogen, alkoxycarbonyl, alkyl, alkylcarbonyl, alkoxysulfonyl, alkylsulfonyl, aryl, arylalkyl, arylcarbonyl, cycloalkyl, cycloalkylalkyl, cycloalkylcarbonyl, formyl, heteroaryl, heteroarylalkyl, heteroarylcarbonyl, heterocycle, heterocyclealkyl, and heterocyclecarbonyl;
R₄ is a member selected from the group consisting of alkenyl, alkenyloxy, alkenyloxyalkyl, alkoxy, alkoxyalkoxy, alkoxyalkoxyalkyl, alkoxyalkyl, alkoxycarbonyl, alkoxycarbonylalkyl, alkoxysulfonyl, alkyl, alkylcarbonyl, alkylcarbonylalkyl, alkylcarbonyloxy, alkylsulfinyl, alkylsulfinylalkyl, alkylsulfonyl, alkylsulfonylalkyl, alkylthio, alkylthioalkyl, alkynyl, alkynyloxy, alkynyloxyalkyl, aryl, arylalkoxy, arylalkoxyalkyl, arylalkyl, arylalkylthio, arylalkylthioalkyl, aryloxy, aryloxyalkyl, arylthio, arylthioalkyl, carboxy, carboxyalkyl, cyano, cyanoalkyl, cycloalkenyl, cycloalkenylalkoxy, cycloalkenylalkoxyalkyl, cycloalkenylalkyl, cycloalkenylalkylthio, cycloalkenylalkylthioalkyl, cycloalkenyloxy, cycloalkenyloxyalkyl, cycloalkenylthio, cycloalkenylthioalkyl, cycloalkyl, cycloalkylalkoxy, cycloalkylalkoxyalkyl, cycloalkylalkyl, cycloalkylalkylthio, cycloalkylalkylthioalkyl, cycloalkyloxy, cycloalkyloxyalkyl, cycloalkylthio, cycloalkylthioalkyl, formyl, formylalkyl, haloalkoxy, haloalkyl, halogen, heteroaryl, heteroarylalkoxy, heteroarylalkoxyalkyl, heteroarylalkyl, heteroarylalkylthio, heteroarylalkylthioalkyl, heteroaryloxy, heteroaryloxyalkyl, heteroarylthio, heteroarylthioalkyl, heterocycle, heterocyclealkoxy, heterocyclealkoxyalkyl, heterocyclealkyl, heterocyclealkylthio, heterocyclealkylthioalkyl, heterocycleoxy, heterocycleoxyalkyl, heterocyclethio, heterocyclethioalkyl, hydroxy, hydroxyalkyl, mercapto, nitro, —NR_{G}R_{H}, (NR_{G}R_{H})alkyl, (NR_{G}R_{H})carbonyl, and (NR_{G}R_{H})sulfonyl;
R_{G} and R_{H} are each independently a member selected from the group consisting of hydrogen, alkoxyalkyl, alkoxyalkylcarbonyl, alkoxycarbonyl, alkoxysulfonyl, alkyl, alkylcarbonyl, alkylsulfonyl, alkylthioalkyl, alkylthioalkylcarbonyl, alkylthiocarbonyl, aryl, arylalkoxyalkyl, arylalkyl, arylcarbonyl, cycloalkyl, cycloalkylalkoxyalkyl, cycloalkyl, cycloalkylalkyl, cycloalkylcarbonyl, formyl, heteroaryl, heteroarylalkoxyalkyl, heteroarylalkyl, heteroarylcarbonyl, heterocycle, heterocyclealkoxyalkyl, heterocyclealkyl, heterocyclecarbonyl, (NZ₃Z₄)alkyl, and (NZ₃Z₄)carbonyl; and
Z₃ and Z₄ are each independently a member selected from the group consisting of hydrogen, alkoxycarbonyl, alkoxysulfonyl, alkyl, alkylcarbonyl, alkylsulfonyl, aryl, arylalkyl, arylcarbonyl, cycloalkyl, cycloalkylalkyl, cycloalkylcarbonyl, formyl, heteroaryl, heteroarylalkyl, heteroarylcarbonyl, heterocycle, heterocyclealkyl, and heterocyclecarbonyl.

According to another embodiment, the present invention is directed to a compound of formula (Ib) wherein R₃ is a member selected from the group consisting of alkoxyalkoxyalkyl, alkoxyalkyl, arylalkoxyalkyl, arylalkyl, aryloxyalkyl, cycloalkylalkoxyalkyl, heterocycle, heterocyclealkoxyalkyl, and (NR_{E}R_{F})carbonylalkyl; R_{E} is a member selected from the group consisting of hydrogen and alkyl; R_{F} is a member selected from the group consisting of hydrogen, alkyl, aryl, and arylalkyl; R₄ is a member selected from the group consisting of arylalkoxy, heteroarylalkoxy, —NR_{G}R_{H}, and (NR_{G}R_{H})alkyl; R_{G} is a member selected from the group consisting of hydrogen, alkyl, alkycarbonyl, and formyl; R_{H} is a member selected from the group consisting of alkoxyalkylcarbonyl, alkyl, alkylcarbonyl, aryl, arylalkoxyalkyl, arylalkyl, arylcarbonyl, cycloalkyl, cycloalkyl, cycloalkylalkyl, heteroaryl, heteroarylalkyl, heterocyclealkyl, (NZ₃Z₄)alkyl, and (NZ₃Z₄)carbonyl; Z₃ is hydrogen; and Z₄ is a member selected from the group consisting of hydrogen and alkoxycarbonyl.

According to another embodiment, the present invention is directed to a compound of formula (Ib) wherein R₃ is a member selected from the group consisting of alkoxyalkoxyalkyl and alkoxyalkyl; R₄ is a member selected from the group consisting of arylalkoxy, heteroarylalkoxy, —NR_{G}R_{H}, and (NR_{G}R_{H})alkyl; R_{G} is a member selected from the group consisting of hydrogen, alkyl, alkycarbonyl, and formyl; R_{H} is a member selected from the group consisting of alkoxyalkylcarbonyl, alkyl, alkylcarbonyl, aryl, arylalkoxyalkyl, arylalkyl, arylcarbonyl, cycloalkyl, cycloalkyl, cycloalkylalkyl, heteroaryl, heteroarylalkyl, heterocyclealkyl, (NZ₃Z₄)alkyl, and (NZ₃Z₄)carbonyl; Z₃ is hydrogen; and Z₄ is a member selected from the group consisting of hydrogen and alkoxycarbonyl.

According to another embodiment, the present invention is directed to a compound of formula (Ib) wherein R₃ is a member selected from the group consisting of arylalkoxyalkyl, arylalkyl, and aryloxyalkyl; R₄ is a member selected from the group consisting of arylalkoxy, heteroarylalkoxy, —NR_{G}R_{H}, and (NR_{G}R_{H})alkyl; R_{G} is a member selected from the group consisting of hydrogen, alkyl, alkycarbonyl, and formyl; R_{H} is a member selected from the group consisting of alkoxyalkylcarbonyl, alkyl, alkylcarbonyl, aryl, arylalkoxyalkyl, arylalkyl, arylcarbonyl, cycloalkyl, cycloalkyl, cycloalkylalkyl, heteroaryl, heteroarylalkyl, heterocyclealkyl, (NZ₃Z₄)alkyl, and (NZ₃Z₄)carbonyl; Z₃ is hydrogen; and Z₄ is a member selected from the group consisting of hydrogen and alkoxycarbonyl.

According to another embodiment, the present invention is directed to a compound of formula (Ib) wherein R₃ is cycloalkylalkoxyalkyl; R₄ is a member selected from the group consisting of arylalkoxy, heteroarylalkoxy, —NR_{G}R_{H}, and (NR_{G}R_{H})alkyl; R_{G} is a member selected from the group consisting of hydrogen, alkyl, alkycarbonyl, and formyl; R_{H} is a member selected from the group consisting of alkoxyalkylcarbonyl, alkyl, alkylcarbonyl, aryl, arylalkoxyalkyl, arylalkyl, arylcarbonyl, cycloalkyl, cycloalkyl, cycloalkylalkyl, heteroaryl, heteroarylalkyl, heterocyclealkyl, (NZ₃Z₄)alkyl, and (NZ₃Z₄)carbonyl; Z₃ is hydrogen; and Z₄ is a member selected from the group consisting of hydrogen and alkoxycarbonyl.

According to another embodiment, the present invention is directed to a compound of formula (Ib) wherein R₃ is a member selected from the group consisting of heterocycle and heterocyclealkoxyalkyl; R₄ is a member selected from the group consisting of arylalkoxy, heteroarylalkoxy, —NR_{G}R_{H}, and (NR_{G}R_{H})alkyl; R_{G} is a member selected from the group consisting of hydrogen, alkyl, alkycarbonyl, and formyl; R_{H} is a member selected from the group consisting of alkoxyalkylcarbonyl, alkyl, alkylcarbonyl, aryl, arylalkoxyalkyl, arylalkyl, arylcarbonyl, cycloalkyl, cycloalkyl, cycloalkylalkyl, heteroaryl, heteroarylalkyl, heterocyclealkyl, (NZ₃Z₄)alkyl, and (NZ₃Z₄)carbonyl; Z₃ is hydrogen; and Z₄ is a member selected from the group consisting of hydrogen and alkoxycarbonyl.

According to another embodiment, the present invention is directed to a compound of formula (Ib) wherein R₃ is (NR_{E}R_{F})carbonylalkyl; R_{E} is a member selected from the group consisting of hydrogen and alkyl; R_{F} is a member selected from the group consisting of hydrogen, alkyl, aryl, and arylalkyl; R₄ is a member selected from the group consisting of arylalkoxy, heteroarylalkoxy, —NR_{G}R_{H}, and (NR_{G}R_{H})alkyl; R_{G} is a member selected from the group consisting of hydrogen, alkyl, alkycarbonyl, and formyl; R_{H} is a member selected from the group consisting of alkoxyalkylcarbonyl, alkyl, alkylcarbonyl, aryl, arylalkoxyalkyl, arylalkyl, arylcarbonyl, cycloalkyl, cycloalkyl, cycloalkylalkyl, heteroaryl, heteroarylalkyl, heterocyclealkyl, (NZ₃Z₄)alkyl, and (NZ₃Z₄)carbonyl; Z₃ is hydrogen; and Z₄ is a member selected from the group consisting of hydrogen and alkoxycarbonyl.

According to another embodiment, the present invention is directed to a pharmaceutical composition comprising a therapeutically effective amount of a compound of formula (Ib) and a pharmaceutically suitable carrier.

According to another embodiment, the present invention is directed to a method of treating a disorder regulated by ghrelin receptors in a mammal, comprising administering a compound of formula (Ib).

According to another embodiment, the present invention is directed to a method of treating anorexia, cancer cachexia, eating disorders, age-related decline in body composition, weight gain, obesity, or diabetes mellitus in a mammal comprising administration of a compound of formula (Ib).

According to an embodiment of the present invention, there is disclosed a method of treating a disorder regulated by ghrelin receptors in a mammal comprising administering to the mammal a therapeutically effective amount of a compound of formula (II) 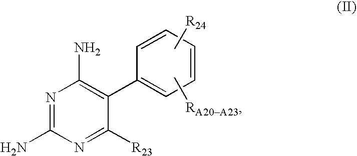
or a therapeutically suitable salt or prodrug thereof, wherein
R₂₃ is a member selected from the group consisting of hydrogen, alkyl, haloalkyl, cyano, and (NR₂₅R₂₆)carbonyl;
R₂₄ is a member selected from the group consisting of alkenyl, alkenyloxy, alkenyloxyalkyl, alkoxy, alkoxyalkoxy, alkoxyalkoxyalkyl, alkoxyalkyl, alkoxycarbonyl, alkoxycarbonylalkyl, alkoxysulfonyl, alkyl, alkylcarbonyl, alkylcarbonylalkyl, alkylcarbonyloxy, alkylsulfinyl, alkylsulfinylalkyl, alkylsulfonyl, alkylsulfonylalkyl, alkylthio, alkylthioalkyl, alkynyl, alkynyloxy, alkynyloxyalkyl, aryl, arylalkoxy, arylalkoxyalkyl, arylalkyl, arylalkylthio, arylalkylthioalkyl, aryloxy, aryloxyalkyl, arylthio, arylthioalkyl, carboxy, carboxyalkyl, cyano, cyanoalkyl, cycloalkenyl, cycloalkenylalkoxy, cycloalkenylalkoxyalkyl, cycloalkenylalkyl, cycloalkenylalkylthio, cycloalkenylalkylthioalkyl, cycloalkenyloxy, cycloalkenyloxyalkyl, cycloalkenylthio, cycloalkenylthioalkyl, cycloalkyl, cycloalkylalkoxy, cycloalkylalkoxyalkyl, cycloalkylalkyl, cycloalkylalkylthio, cycloalkylalkylthioalkyl, cycloalkyloxy, cycloalkyloxyalkyl, cycloalkylthio, cycloalkylthioalkyl, formyl, formylalkyl, haloalkoxy, haloalkyl, halogen, heteroaryl, heteroarylalkoxy, heteroarylalkoxyalkyl, heteroarylalkyl, heteroarylalkylthio, heteroarylalkylthioalkyl, heteroaryloxy, heteroaryloxyalkyl, heteroarylthio, heteroarylthioalkyl, heterocycle, heterocyclealkoxy, heterocyclealkoxyalkyl, heterocyclealkyl, heterocyclealkylthio, heterocyclealkylthioalkyl, heterocycleoxy, heterocycleoxyalkyl, heterocyclethio, heterocyclethioalkyl, hydroxy, hydroxyalkyl, mercapto, nitro, —NR₂₇R₂₈, (NR₂₇R₂₈)alkyl, (NR₂₇R₂₈)carbonyl, and (NR₂₇R₂₈)sulfonyl;
R₂₅ and R₂₆ are each independently a member selected from the group consisting of hydrogen, alkyl, and alkylcarbonyl;
R₂₇ and R₂₈ are each independently a member selected from the group consisting of hydrogen, alkoxyalkyl, alkoxyalkylcarbonyl, alkoxycarbonyl, alkoxysulfonyl, alkyl, alkylcarbonyl, alkylsulfonyl, alkylthioalkyl, alkylthioalkylcarbonyl, alkylthiocarbonyl, aryl, arylalkoxyalkyl, arylalkyl, arylcarbonyl, cycloalkyl, cycloalkylalkoxyalkyl, cycloalkylalkyl, cycloalkylcarbonyl, formyl, heteroaryl, heteroarylalkoxyalkyl, heteroarylalkyl, heteroarylcarbonyl, heterocycle, heterocyclealkoxyalkyl, heterocyclealkyl, heterocyclecarbonyl, (NZ₂₃Z₂₄)alkyl, and (NZ₂₃Z₂₄)carbonyl;
Z₂₃ and Z₂₄ are each independently a member selected from the group consisting of hydrogen, alkoxycarbonyl, alkoxysulfonyl, alkyl, alkylcarbonyl, alkylsulfonyl, aryl, arylalkyl, arylcarbonyl, cycloalkyl, cycloalkylalkyl, cycloalkylcarbonyl, formyl, heteroaryl, heteroarylalkyl, heteroarylcarbonyl, heterocycle, heterocyclealkyl, and heterocyclecarbonyl;

R_{A20}, R_{A21,} R_{A22}, and R_{A23} are each independently a member selected from the group consisting of hydrogen, alkenyl, alkenyloxy, alkoxy, alkoxyalkoxy, alkoxyalkoxyalkyl, alkoxyalkyl, alkoxycarbonyl, alkoxycarbonylalkyl, alkoxysulfonyl, alkyl, alkylcarbonyl, alkylcarbonylalkyl, alkylcarbonyloxy, alkylsulfinyl, alkylsulfinylalkyl, alkylsulfonyl, alkylsulfonylalkyl, alkylthio, alkylthioalkyl, alkynyl, aryl, carboxy, carboxyalkyl, cyano, cyanoalkyl, cycloalkyl, formyl, haloalkoxy, haloalkyl, halogen, heteroaryl, heterocycle, hydroxy, hydroxyalkyl, mercapto, nitro, —NR₃₀R₃₁, (NR₃₀R₃₁)alkyl, (NR₃₀R₃₁)carbonyl, and (NR₃₀R₃₁)sulfonyl; and
R₃₀ and R₃₁ are each independently a member selected from the group consisting of hydrogen, alkoxycarbonyl, alkyl, alkylcarbonyl, alkoxysulfonyl, alkylsulfonyl, aryl, arylalkyl, and formyl.

According to another embodiment, the present invention is directed to a pharmaceutical composition comprising a therapeutically effective amount of a compound of formula (II) and a pharmaceutically suitable carrier.

According to another embodiment, the present invention is directed to a method of treating anorexia, cancer cachexia, eating disorders, age-related decline in body composition, weight gain, obesity, or diabetes mellitus in a mammal comprising administering to the mammal a therapeutically effective amount of a compound of formula (II). 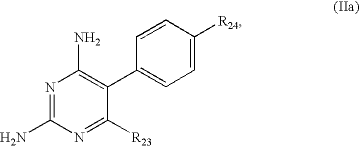
or a therapeutically suitable salt or prodrug thereof, wherein
R₂₃ is a member selected from the group consisting of hydrogen, alkyl, haloalkyl, cyano, and (NR₂₅R₂₆)carbonyl;
R₂₄ is a member selected from the group consisting of alkenyl, alkenyloxy, alkenyloxyalkyl, alkoxy, alkoxyalkoxy, alkoxyalkoxyalkyl, alkoxyalkyl, alkoxycarbonyl, alkoxycarbonylalkyl, alkoxysulfonyl, alkyl, alkylcarbonyl, alkylcarbonylalkyl, alkylcarbonyloxy, alkylsulfinyl, alkylsulfinylalkyl, alkylsulfonyl, alkylsulfonylalkyl, alkylthio, alkylthioalkyl, alkynyl, alkynyloxy, alkynyloxyalkyl, aryl, arylalkoxy, arylalkoxyalkyl, arylalkyl, arylalkylthio, arylalkylthioalkyl, aryloxy, aryloxyalkyl, arylthio, arylthioalkyl, carboxy, carboxyalkyl, cyano, cyanoalkyl, cycloalkenyl, cycloalkenylalkoxy, cycloalkenylalkoxyalkyl, cycloalkenylalkyl, cycloalkenylalkylthio, cycloalkenylalkylthioalkyl, cycloalkenyloxy, cycloalkenyloxyalkyl, cycloalkenylthio, cycloalkenylthioalkyl, cycloalkyl, cycloalkylalkoxy, cycloalkylalkoxyalkyl, cycloalkylalkyl, cycloalkylalkylthio, cycloalkylalkylthioalkyl, cycloalkyloxy, cycloalkyloxyalkyl, cycloalkylthio, cycloalkylthioalkyl, formyl, formylalkyl, haloalkoxy, haloalkyl, halogen, heteroaryl, heteroarylalkoxy, heteroarylalkoxyalkyl, heteroarylalkyl, heteroarylalkylthio, heteroarylalkylthioalkyl, heteroaryloxy, heteroaryloxyalkyl, heteroarylthio, heteroarylthioalkyl, heterocycle, heterocyclealkoxy, heterocyclealkoxyalkyl, heterocyclealkyl, heterocyclealkylthio, heterocyclealkylthioalkyl, heterocycleoxy, heterocycleoxyalkyl, heterocyclethio, heterocyclethioalkyl, hydroxy, hydroxyalkyl, mercapto, nitro, —NR₂₇R₂₈, (NR₂₇R₂₈)alkyl, (NR₂₇R₂₈)carbonyl, and (NR₂₇R₂₈)sulfonyl;
R₂₅ and R₂₆ are each independently a member selected from the group consisting of hydrogen, alkyl, and alkylcarbonyl;
R₂₇ and R₂₈ are each independently a member selected from the group consisting of hydrogen, alkoxyalkyl, alkoxyalkylcarbonyl, alkoxycarbonyl, alkoxysulfonyl, alkyl, alkylcarbonyl; alkylsulfonyl, alkylthioalkyl, alkylthioalkylcarbonyl, alkylthiocarbonyl, aryl, arylalkoxyalkyl, arylalkyl, arylcarbonyl, cycloalkyl, cycloalkylalkoxyalkyl, cycloalkylalkyl, cycloalkylcarbonyl, formyl, heteroaryl, heteroarylalkoxyalkyl, heteroarylalkyl, heteroarylcarbonyl, heterocycle, heterocyclealkoxyalkyl, heterocyclealkyl, heterocyclecarbonyl, (NZ₂₃Z₂₄)alkyl, and (NZ₂₃Z₂₄)carbonyl; and
Z₂₃ and Z₂₄ are each independently a member selected from the group consisting of hydrogen, alkoxycarbonyl, alkoxysulfonyl, alkyl, alkylcarbonyl, alkylsulfonyl, aryl, arylalkyl, arylcarbonyl, cycloalkyl, cycloalkylalkyl, cycloalkylcarbonyl, formyl, heteroaryl, heteroarylalkyl, heteroarylcarbonyl, heterocycle, heterocyclealkyl, and heterocyclecarbonyl.

According to another embodiment, the present invention is directed to a method of treating a disorder regulated by ghrelin receptors in a mammal comprising administering to the mammal a therapeutically effective amount of a compound of formula (IIa) wherein R₂₃ is alkyl; R₂₄ is —NR₂₇R₂₈; R₂₇ is a member selected from the group consisting of hydrogen, alkyl, alkylcarbonyl, and formyl; and R₂₈ is a member selected from the group consisting of arylalkoxyalkyl and arylalkyl;

According to another embodiment, the present invention is directed to a method of treating a disorder regulated by ghrelin receptors in a mammal comprising administering to the mammal a therapeutically effective amount of a compound of formula (IIa) wherein R₂₃ is alkyl wherein the alkyl is selected from the group consisting of ethyl and propyl; R₂₄ is —NR₂₇R₂₈; R₂₇ is hydrogen; and R₂₈ is arylalkyl wherein the arylalkyl is selected from the group consisting of 4-chlorobenzyl, 4-cyanobenzyl, 3,4-dichlorobenzyl, and 4-nitrobenzyl;

According to another embodiment, the present invention is directed to a method of treating a disorder regulated by ghrelin receptors in a mammal comprising administering to the mammal a therapeutically effective amount of a compound of formula (IIa) wherein R₂₃ is alkyl wherein the alkyl is selected from the group consisting of ethyl and propyl; R₂₄ is —NR₂₇R₂₈; R₂₇ is hydrogen; and R₂₈ is arylalkoxyalkyl wherein the arylalkoxyalkyl is 2-(benzyloxy)ethyl.

According to another embodiment, the present invention is directed to a pharmaceutical composition comprising a therapeutically effective amount of a compound of formula (IIa) and a pharmaceutically suitable carrier.

According to another embodiment, the present invention is directed to a method of treating anorexia, cancer cachexia, eating disorders, age-related decline in body composition, weight gain, obesity, or diabetes mellitus in a mammal comprising administering to the mammal a therapeutically effective amount of a compound of formula (IIa).

### Definitions

As used throughout this specification and the appended claims, the following terms have the following meanings:

The term “alkenyl” as used herein, means a straight or branched chain hydrocarbon containing from 2 to 10 carbons and containing at least one carbon-carbon double bond formed by the removal of two hydrogens. Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, and 3-decenyl.

The term “alkenyloxy” as used herein, means an alkenyl group, as defined herein, appended to the parent molecular moiety through an oxygen atom.

The term “alkenyloxyalkyl” as used herein, means an alkenyloxy group, as defined herein, appended to the parent molecular moiety through an alkyl group, as defined herein.

The term “alkoxy” as used herein, means an alkyl group, as defined herein, appended to the parent molecular moiety through an oxygen atom. Representative examples of alkoxy include, but are not limited to, methoxy, ethoxy, propoxy, 2-propoxy, n-butoxy, tert-butoxy, pentyloxy, and hexyloxy.

The term “alkoxyalkoxy” as used herein, means an alkoxy group, as defined herein, appended to the parent molecular moiety through an alkoxy group. Representative example of alkoxyalkoxy include, but are not limited to, 2-(methoxy)ethoxy, 2-(ethoxy)ethoxy, 3-(methoxy)propoxy, and 2-(n-butoxy)ethoxy.

The term “alkoxyalkoxyalkyl” as used herein, means an alkoxyalkoxy group, as defined herein, appended to the parent molecular moiety through an alkyl group, as defined herein. Representative examples of alkoxyalkoxyalkyl include, but are not limited to, 2-(methoxy)ethoxymethyl, 2-(ethoxy)ethoxymethyl, 3-(methoxy)propoxymethyl, 2-(n-butoxy)ethoxymethyl, and 2-(tert-butoxy)ethoxymethyl.

The term “alkoxyalkyl” as used herein, means an alkoxy group, as defined herein, appended to the parent molecular moiety through an alkyl group, as defined herein. Representative examples of alkoxyalkyl include, but are not limited to, n-butoxymethyl, tert-butoxymethyl, 2-(ethoxy)ethyl, 2-methoxyethyl, and methoxymethyl.

The term “alkoxyalkylcarbonyl” as used herein, means an alkoxyalkyl group, as defined herein, appended to the parent molecular moiety through a carbonyl group, as defined herein. Representative examples of alkoxyalkylcarbonyl include, but are not limited to, n-butoxymethylcarbonyl, tert-butoxymethylcarbonyl, 2-(ethoxy)ethylcarbonyl, 2-methoxyethylcarbonyl, and methoxymethylcarbonyl.

The term “alkoxycarbonyl” as used herein, means an alkoxy group, as defined herein, appended to the parent molecular moiety through a carbonyl group, as defined herein. Representative examples of alkoxycarbonyl include, but are not limited to, methoxycarbonyl, ethoxycarbonyl, and tert-butoxycarbonyl.

The term “alkoxycarbonylalkyl” as used herein, means an alkoxycarbonyl group, as defined herein, appended to the parent molecular moiety through a alkyl group, as defined herein.

The term “alkoxysulfonyl” as used herein, means an alkoxy group, as defined herein, appended to the parent molecular moiety through a sulfonyl group, as defined herein. Representative examples of alkoxysulfonyl include, but are not limited to, methoxysulfonyl, ethoxysulfonyl, and tert-butoxysulfonyl.

The term “alkyl” as used herein, means a straight or branched chain hydrocarbon containing from 1 to 10 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl.

The term “alkylcarbonyl” as used herein, means an alkyl group, as defined herein, appended to the parent molecular moiety through a carbonyl group, as defined herein. Representative examples of alkylcarbonyl include, but are not limited to, acetyl, 1-oxopropyl, 2,2-dimethyl-1-oxopropyl, 1-oxobutyl, and 1-oxopentyl.

The term “alkylcarbonylalkyl” as used herein, means an alkylcarbonyl group, as defined herein, appended to the parent molecular moiety through an alkyl group, as defined herein. Representative examples of alkylcarbonylalkyl include, but are not limited to, 2-oxopropyl, 3-oxobutyl, 3-oxopentyl, and 4-oxopentyl.

The term “alkylcarbonyloxy” as used herein, means an alkylcarbonyl group, as defined herein, appended to the parent molecular moiety through an oxygen atom, as defined herein. Representative examples of alkylcarbonyloxy include, but are not limited to, acetyloxy, propionyloxy, 3-oxobutyl, and butyryloxy.

The term “alkylsulfinyl” as used herein, means an alkyl group, as defined herein, appended to the parent molecular moiety through a sulfinyl group, as defined herein. Representative examples of alkylsulfinyl include, but are not limited to, methylsulfinyl and ethylsulfinyl.

The term “alkylsulfinylalkyl” as used herein, means an alkylsulfinyl group, as defined herein, appended to the parent molecular moiety through an alkyl group, as defined herein. Representative examples of alkylsulfinylalkyl include, but are not limited to, methylsulfinylmethyl and ethylsulfinylmethyl.

The term “alkylsulfonyl” as used herein, means an alkyl group, as defined herein, appended to the parent molecular moiety through a sulfonyl group, as defined herein. Representative examples of alkylsulfonyl include, but are not limited to, methylsulfonyl and ethylsulfonyl.

The term “alkylsulfonylalkyl” as used herein, means an alkylsulfonyl group, as defined herein, appended to the parent molecular moiety through an alkyl group, as defined herein. Representative examples of alkylsulfonyl include, but are not limited to, methylsulfonylmethyl and ethylsulfonylmethyl.

The term “alkylthio” as used herein, means an alkyl group, as defined herein, appended to the parent molecular moiety through a sulfur atom. Representative examples of alkylthio include, but are not limited to, methylthio and ethylthio.

The term “alkylthioalkyl” as used herein, means an alkylthio group, as defined herein, appended to the parent molecular moiety through an alkyl group, as defined herein. Representative examples of alkylthioalkyl include, but are not limited to, methylthiomethyl and ethylthiomethyl.

The term “alkylthioalkylcarbonyl” as used herein, means an alkylthioalkyl group, as defined herein, appended to the parent molecular moiety through a carbonyl group, as defined herein. Representative examples of alkylthioalkylcarbonyl include, but are not limited to, methylthiomethylcarbonyl and ethylthiomethylcarbonyl.

The term “alkylthiocarbonyl” as used herein, means an alkylthio group, as defined herein, appended to the parent molecular moiety through a carbonyl group, as defined herein. Representative examples of alkylthiocarbonyl include, but are not limited to, methylthiocarbonyl and ethylthiocarbonyl.

The term “alkynyl” as used herein, means a straight or branched chain hydrocarbon group containing from 2 to 10 carbon atoms and containing at least one carbon-carbon triple bond. Representative examples of alkynyl include, but are not limited, to acetylenyl, 1-propynyl, 2-propynyl, 3-butynyl, 2-pentynyl, and 1-butynyl.

The term “alkynyloxy” as used herein, means an alkynyl group, as defined herein, appended to the parent molecular moiety through an oxygen atom. Representative examples of alkynyloxy include, but are not limited, to but-3-ynyloxy and hex-4-ynyloxy.

The term “alkynyloxyalkyl” as used herein, means an alkynyloxy group, as defined herein, appended to the parent molecular moiety through an alkyl group, as defined herein. Representative examples of alkynyloxyalkyl include, but are not limited, to but-3-ynyloxymethyl and hex-4-ynyloxymethyl.

The term “aryl” as used herein, means a phenyl group, or a bicyclic or a tricyclic fused ring system wherein one or more of the fused rings is a phenyl group. Bicyclic fused ring systems are exemplified by a phenyl group fused to a cycloalkyl group, as defined herein, or another phenyl group. Tricyclic fused ring systems are exemplified by a bicyclic fused ring system fused to a cycloalkyl group, as defined herein, or another phenyl group. Representative examples of aryl include, but are not limited to, anthracenyl, azulenyl, fluorenyl, indanyl, indenyl, naphthyl, phenyl, and tetrahydronaphthyl.

The aryl groups of this invention can be substituted with 0, 1, 2, 3, 4, or 5 substituents independently a member selected from the group consisting of alkenyl, alkoxy, alkoxyalkyl, alkoxycarbonyl, alkoxycarbonylalkyl, alkyl, alkylcarbonyl, alkylcarbonylalkyl, alkylcarbonyloxy, alkylsulfonyl, alkylthio, alkynyl, arylcarbonyl, arylsulfonyl, carboxy, carboxyalkyl, cyano, cyanoalkyl, formyl, haloalkoxy, haloalkyl, haloalkylcarbonyl, haloalkylsulfonyl, halogen, hydroxy, hydroxyalkyl, hydroxyhaloalkyl, mercapto, nitro, —NZ₅Z₆ and (NZ₅Z₆)alkyl. Representative examples include, but are not limited to, 2-bromophenyl, 2-chlorophenyl, 3-chlorophenyl, 4-chlorophenyl, 3-cyanophenyl, 4-cyanophenyl, 2,3-dichlorophenyl, 3,4-dichlorophenyl, 2,5-dichlorophenyl, 2,4-dimethylphenyl, 3,5-dimethylphenyl, 2-fluoro-3-methylphenyl, 2-fluorophenyl, 3-fluorophenyl, 4-fluorophenyl, 2-methoxyphenyl, 3-methoxyphenyl, 4-methoxyphenyl, 2-methylphenyl, 3-methylphenyl, 4-(methylthio)phenyl, 4-nitrophenyl, 4-(trifluoromethoxy)phenyl, and 3-(trifluoromethyl)phenyl.

The term “arylalkoxy” as used herein, means an aryl group, as defined herein, appended to the parent molecular moiety through an alkoxy group, as defined herein. Representative examples of arylalkoxy include, but are not limited to, benzyloxy, 2-bromobenzyloxy, 2-chlorobenzyloxy, 3-chlorobenzyloxy, 4-chlorobenzyloxy, 2-(4-chlorophenyl)ethoxy, 3-cyanobenzyloxy, 4-cyanobenzyloxy, 2,3-dichlorobenzyloxy, 2,5-dichlorobenzyloxy, 2,4-dimethylbenzyloxy, 3,5-dimethylbenzyloxy, 2-fluoro-3-methylbenzyloxy, 2-fluorobenzyloxy, 4-fluorobenzyloxy, 2-methoxybenzyloxy, 3-methoxybenzyloxy, 4-methoxybenzyloxy, 2-methylbenzyloxy, 3-methylbenzyloxy, 4-(methylthio)benzyloxy, 4-nitrobenzyloxy, 4-(trifluoromethoxy)benzyloxy, and 3-(trifluoromethyl)benzyloxy.

The term “arylalkoxyalkyl” as used herein, means an arylalkoxy group, as defined herein, appended to the parent molecular moiety through an alkyl group, as defined herein. Representative examples of arylalkoxyalkyl include, but are not limited to, benzyloxymethyl, 2-bromobenzyloxymethyl, 2-chlorobenzyloxymethyl, 3-chlorobenzyloxymethyl, 4-chlorobenzyloxymethyl, 4-cyanobenzyloxymethyl, 2,3-dichlorobenzyloxymethyl, 2,5-dichlorobenzyloxymethyl, 2,4-dimethylbenzyloxymethyl, 3,5-dimethylbenzyloxymethyl, 2-fluoro-3-methylbenzyloxymethyl, 2-fluorobenzyloxymethyl, 4-fluorobenzyloxymethyl, 2-methoxybenzyloxymethyl, 3-methoxybenzyloxymethyl, 4-methoxybenzyloxymethyl, 2-methylbenzyloxymethyl, 3-methylbenzyloxymethyl, 4-(methylthio)benzyloxymethyl, 4-nitrobenzyloxymethyl, 4-(trifluoromethoxy)benzyloxymethyl, and 3-(trifluoromethyl)benzyloxymethyl.

The term “arylalkyl” as used herein, means an aryl group, as defined herein, appended to the parent molecular moiety through an alkyl group, as defined herein. Representative examples of arylalkyl include, but are not limited to, benzyl, 2-phenylethyl, 1-phenylethyl, 3-phenylpropyl, 4-phenylbutyl, 2-naphth-2-ylethyl, 2-bromobenzyl, 4-cyanobenzyl, 1-(4-cyanophenyl)ethyl, 2-chlorobenzyl, 3-chlorobenzyl, 4-chlorobenzyl, 1-(4-chlorophenyl)ethyl, 2-(4-chlorophenyl)ethyl, 2,3-dichlorobenzyl, 2,5-dichlorobenzyl, 2,4-dimethylbenzyl, 3,5-dimethylbenzyl, 2-fluoro-3-methylbenzyl, 2-fluorobenzyl, 4-fluorobenzyl, 2-methoxybenzyl, 3-methoxybenzyl, 4-methoxybenzyl, 2-methylbenzyl, 3-methylbenzyl, 4-(methylthio)benzyl, 4-nitrobenzyl, 1-(4-nitrophenyl)ethyl, 2-(4-chlorophenyl)ethyl, 4-(trifluoromethoxy)benzyl, and 3-(trifluoromethyl)benzyl.

The term “arylalkylthio” as used herein, means an arylalkyl group, as defined herein, appended to the parent molecular moiety through a sulfur atom. Representative examples of arylalkylthio include, but are not limited to, benzylthio, 2-phenylethylthio, 1-phenylethylthio, 3-phenylpropylthio, 4-phenylbutylthio, 2-naphth-2-ylethylthio, 2-bromobenzylthio, 4-cyanobenzylthio, 1-(4-cyanophenyl)ethyl, 2-chlorobenzylthio, 3-chlorobenzylthio, 4-chlorobenzylthio, 1-(4-chlorophenyl)ethylthio, 2-(4-chlorophenyl)ethylthio, 2,3-dichlorobenzylthio, 2,5-dichlorobenzylthio, 2,4-dimethylbenzylthio, 3,5-dimethylbenzylthio, 2-fluoro-3-methylbenzylthio, 2-fluorobenzylthio, 4-fluorobenzylthio, 2-methoxybenzylthio, 3-methoxybenzylthio, 4-methoxybenzylthio, 2-methylbenzylthio, 3-methylbenzylthio, 4-(methylthio)benzylthio, 4-nitrobenzylthio, 1-(4-nitrophenyl)ethylthio, 2-(4-chlorophenyl)ethylthio, 4-(trifluoromethoxy)benzylthio, and 3-(trifluoromethyl)benzylthio.

The term “arylalkylthioalkyl” as used herein, means an arylalkylthio group, as defined herein, appended to the parent molecular moiety through an alkyl group, as defined herein. Representative examples of arylalkylthio include, but are not limited to, benzylthiomethyl, 2-phenylethylthiomethyl, 1-phenylethylthiomethyl, 3-phenylpropylthiomethyl, 4-phenylbutylthiomethyl, 2-naphth-2-ylethylthiomethyl, 2-bromobenzylthiomethyl, 4-cyanobenzylthiomethyl, 1-(4-cyanophenyl)ethylmethyl, 2-chlorobenzylthiomethyl, 3-chlorobenzylthiomethyl, 4-chlorobenzylthiomethyl, 1-(4-chlorophenyl)ethylthiomethyl, 2-(4-chlorophenyl)ethylthiomethyl, 2,3-dichlorobenzylthiomethyl, 2,5-dichlorobenzylthiomethyl, 2,4-dimethylbenzylthiomethyl, 3,5-dimethylbenzylthiomethyl, 2-fluoro-3-methylbenzylthiomethyl, 2-fluorobenzylthiomethyl, 4-fluorobenzylthiomethyl, 2-methoxybenzylthiomethyl, 3-methoxybenzylthiomethyl, 4-methoxybenzylthiomethyl, 2-methylbenzylthiomethyl, 3-methylbenzylthiomethyl, 4-(methylthio)benzylthiomethyl, 4-nitrobenzylthiomethyl, 1-(4-nitrophenyl)ethylthiomethyl, 2-(4-chlorophenyl)ethylthiomethyl, 4-(trifluoromethoxy)benzylthiomethyl, and 3-(trifluoromethyl)benzylthiomethyl.

The term “arylcarbonyl” as used herein, means an aryl group, as defined herein, appended to the parent molecular moiety through a carbonyl group, as defined herein. Representative examples of arylcarbonyl include, but are not limited to, benzoyl, naphthoyl, 2-bromo benzoyl, 2-chlorobenzoyl, 3-chlorobenzoyl, 4-chlorobenzoyl, 3-cyanobenzoyl, 4-cyanobenzoyl, 2,3-dichlorobenzoyl, 3,4-dichlorobenzoyl, 2,5-dichlorobenzoyl, 2,4-dimethylbenzoyl, 3,5-dimethylbenzoyl, 2-fluoro-3-methylbenzoyl, 2-fluorobenzoyl, 3-fluorobenzoyl, 4-fluorobenzoyl, 2-methoxybenzoyl, 3-methoxybenzoyl, 4-methoxybenzoyl, 2-methylbenzoyl, 3-methylbenzoyl, 4-(methylthio)benzoyl, 4-nitrobenzoyl, 4-(trifluoromethoxy)benzoyl, and 3-(trifluoromethyl)benzoyl.

The term “aryloxy” as used herein, means an aryl group, as defined herein, appended to the parent molecular moiety through an oxygen atom. Representative examples of aryloxy include, but are not limited to, 2-bromophenoxy, 2-chlorophenoxy, 3-chlorophenoxy, 4-chlorophenoxy, 4-cyanophenoxy, 2,3-dichlorophenoxy, 3,4-dichlorophenoxy, 2,5-dichlorophenoxy, 2,4-dimethylphenoxy, 3,5-dimethylphenoxy, 2-fluoro-3-methylphenoxy, 2-fluorophenoxy, 3-fluorophenoxy, 4-fluorophenoxy, 2-methoxyphenoxy, 3-methoxyphenoxy, 4-methoxyphenoxy, 2-methylphenoxy, 3-methylphenoxy, 4-(methylthio)phenoxy, 3-nitrophenoxy, 4-nitrophenoxy, 4-(trifluoromethoxy)phenoxy, and 3-(trifluoromethyl)phenoxy.

The term “aryloxyalkyl” as used herein, means an aryloxy group, as defined herein, appended to the parent molecular moiety through an alkyl group, as defined herein. Representative examples of aryloxyalkyl include, but are not limited to, 2-(2-bromophenoxy)ethyl, 2-(2-chlorophenoxy)ethyl, 3-chlorophenoxymethyl, 4-chlorophenoxymethyl, 4-cyanophenoxymethyl, 2,3-dichlorophenoxymethyl, 3,4-dichlorophenoxymethyl, 2,5-dichlorophenoxymethyl, 2,4-dimethylphenoxymethyl, 3,5-dimethylphenoxymethyl, 2-fluoro-3-methylphenoxymethyl, 2-fluorophenoxymethyl, 3-fluorophenoxymethyl, 4-fluorophenoxymethyl, 2-methoxyphenoxymethyl, 3-methoxyphenoxymethyl, 4-methoxyphenoxymethyl, 2-methylphenoxymethyl, 3-methylphenoxymethyl, 4-(methylthio)phenoxymethyl, 3-nitrophenoxymethyl, 4-nitrophenoxymethyl, 4-(trifluoromethoxy)phenoxymethyl, and 3-(trifluoromethyl)phenoxymethyl.

The term “arylsulfonyl” as used herein, means an aryl group, as defined herein, appended to the parent molecular moiety through a sulfonyl group, as defined herein. Representative examples of arylsulfonyl include, but are not limited to, phenylsulfonyl, naphthylsulfonyl, 2-bromophenylsulfonyl, 2-chlorophenylsulfonyl, 3-chlorophenylsulfonyl, 4-chlorophenylsulfonyl, 3-cyanophenylsulfonyl, 4-cyanophenylsulfonyl, 2,3-dichlorophenylsulfonyl, 3,4-dichlorophenylsulfonyl, 2,5-dichlorophenylsulfonyl, 2,4-dimethylphenylsulfonyl, 3,5-dimethylphenylsulfonyl, 2-fluoro-3-methylphenylsulfonyl, 2-fluorophenylsulfonyl, 3-fluorophenylsulfonyl, 4-fluorophenylsulfonyl, 2-methoxyphenylsulfonyl, 3-methoxyphenylsulfonyl, 4-methoxyphenylsulfonyl, 2-methylphenylsulfonyl, 3-methylphenylsulfonyl, 4-(methylthio)phenylsulfonyl, 4-nitrophenylsulfonyl, 4-(trifluoromethoxy)phenylsulfonyl, and 3-(trifluoromethyl)phenylsulfonyl.

The term “arylthio” as used herein, means an aryl group, as defined herein, appended to the parent molecular moiety through a sulfur atom. Representative examples of arylthio include, but are not limited to, 2-bromophenylthio, 2-chlorophenylthio, 3-chlorophenylthio, 4-chlorophenylthio, 4-cyanophenylthio, 2,3-dichlorophenylthio, 3,4-dichlorophenylthio, 2,5-dichlorophenylthio, 2,4-dimethylphenylthio, 3,5-dimethylphenylthio, 2-fluoro-3-methylphenylthio, 2-fluorophenylthio, 3-fluorophenylthio, 4-fluorophenylthio, 2-methoxyphenylthio, 3-methoxyphenylthio, 4-methoxyphenylthio, 2-methylphenylthio, 3-methylphenylthio, 4-(methylthio)phenylthio, 3-nitrophenylthio, 4-nitrophenylthio, 4-(trifluoromethoxy)phenylthio, and 3-(trifluoromethyl)phenylthio.

The term “arylthioalkyl” as used herein, means an arylthio group, as defined herein, appended to the parent molecular moiety through an alkyl group, as defined herein. Representative examples of arylthioalkyl include, but are not limited to, 2-bromophenylthiomethyl, 2-chlorophenylthiomethyl, 3-chlorophenylthiomethyl, 4-chlorophenylthiomethyl, 4-cyanophenylthiomethyl, 2,3-dichlorophenylthiomethyl, 3,4-dichlorophenylthiomethyl, 2,5-dichlorophenylthiomethyl, 2,4-dimethylphenylthiomethyl, 3,5-dimethylphenylthiomethyl, 2-fluoro-3-methylphenylthiomethyl, 2-fluorophenylthiomethyl, 3-fluorophenylthiomethyl, 4-fluorophenylthiomethyl, 2-methoxyphenylthiomethyl, 3-methoxyphenylthiomethyl, 4-methoxyphenylthiomethyl, 2-methylphenylthiomethyl, 3-methylphenylthiomethyl, 4-(methylthio)phenylthiomethyl, 3-nitrophenylthiomethyl, 4-nitrophenylthiomethyl, 4-(trifluoromethoxy)phenylthiomethyl, and 3-(trifluoromethyl)phenylthiomethyl.

The term “carbonyl” as used herein, means a —C(═O)— group.

The term “carboxy” as used herein, means a —CO₂H group.

The term “carboxyalkyl” as used herein, means a carboxy group, as defined herein, appended to the parent molecular moiety through an alkyl group, as defined herein. Representative examples of carboxyalkyl include, but are not limited to, carboxymethyl, 2-carboxyethyl, and 3-carboxypropyl.

The term “cyano” as used herein, means a —CN group.

The term “cyanoalkyl” as used herein, means a cyano group, as defined herein, appended to the parent molecular moiety through an alkyl group, as defined herein. Representative examples of cyanoalkyl include, but are not limited to, cyanomethyl, 2-cyanoethyl, and 3-cyanopropyl.

The term “cycloalkenyl” as used herein, means a cycloalkyl group, as defined herein, which contains 1 or 2 double bonds. Representative examples of cycloalkenyl include, but are not limited to, cyclopropenyl, cyclobutenyl, cyclopentenyl, cyclohexenyl, cycloheptenyl, and cyclooctenyl.

The cycloalkenyl groups of this invention can be substituted with 0, 1, 2, 3, or 4 substituents independently a member selected from the group consisting of alkenyl, alkoxy, alkoxyalkyl, alkoxycarbonyl, alkoxycarbonylalkyl, alkyl, alkylcarbonyl, alkylcarbonylalkyl, alkylcarbonyloxy, alkylsulfonyl, alkylthio, alkynyl, carboxy, carboxyalkyl, cyano, cyanoalkyl, formyl, halogen, haloalkyl, hydroxy, hydroxyalkyl, mercapto, —NZ₅Z₆ and (NZ₅Z₆)alkyl.

The term “cycloalkenylalkoxy” as used herein, means a cycloalkenyl group, as defined herein, appended to the parent molecular moiety through an alkoxy group, as defined herein. Representative examples of cycloalkenylalkoxy include, but are not limited to, cyclopropenylmethoxy, cyclobutenylmethoxy, cyclopentenylmethoxy, cyclohexenylmethoxy, cycloheptenylmethoxy, and cyclooctenylmethoxy.

The term “cycloalkenylalkoxyalkyl” as used herein, means a cycloalkenylalkoxy group, as defined herein, appended to the parent molecular moiety through an alkyl group, as defined herein. Representative examples of cycloalkenylalkoxyalkyl include, but are not limited to, cyclopropenylmethoxymethyl, cyclobutenylmethoxymethyl, cyclopentenylmethoxymethyl, cyclohexenylmethoxymethyl, cycloheptenylmethoxymethyl, and cyclooctenylmethoxymethyl.

The term “cycloalkenylalkyl” as used herein, means a cycloalkenyl group, as defined herein, appended to the parent molecular moiety through an alkyl group, as defined herein. Representative examples of cycloalkenylalkyl include, but are not limited to, cyclopropenylmethyl, cyclobutenylmethyl, cyclopentenylmethyl, cyclohexenylmethyl, cycloheptenylmethyl, and cyclooctenylmethyl.

The term “cycloalkenylalkylthio” as used herein, means a cycloalkenylalkyl group, as defined herein, appended to the parent molecular moiety through a sulfur atom. Representative examples of cycloalkenylalkylthio include, but are not limited to, cyclopropenylmethylthio, cyclobutenylmethylthio, cyclopentenylmethylthio, cyclohexenylmethylthio, cycloheptenylmethylthio, and cyclooctenylmethylthio.

The term “cycloalkenylalkylthioalkyl” as used herein, means a cycloalkenylalkylthio group, as defined herein, appended to the parent molecular moiety through an alkyl group, as defined herein. Representative examples of cycloalkenylalkylthioalkyl include, but are not limited to, cyclopropenylmethylthiomethyl, cyclobutenylmethylthiomethyl, cyclopentenylmethylthiomethyl, cyclohexenylmethylthiomethyl, cycloheptenylmethylthiomethyl, and cyclooctenylmethylthiomethyl.

The term “cycloalkenyloxy” as used herein, means a cycloalkenyl group, as defined herein, appended to the parent molecular moiety through an oxygen atom. Representative examples of cycloalkenyloxy include, but are not limited to, cyclopropenyloxy, cyclobutenyloxy, cyclopentenyloxy, cyclohexenyloxy, cycloheptenyloxy, and cyclooctenyloxy.

The term “cycloalkenyloxyalkyl” as used herein, means a cycloalkenyloxy group, as defined herein, appended to the parent molecular moiety through an alkyl group, as defined herein. Representative examples of cycloalkenyloxyalkyl include, but are not limited to, cyclopropenyloxymethyl, cyclobutenyloxymethyl, cyclopentenyloxymethyl, cyclohexenyloxymethyl, cycloheptenyloxymethyl, and cyclooctenyloxymethyl.

The term “cycloalkenylthio” as used herein, means a cycloalkenyl group, as defined herein, appended to the parent molecular moiety through a sulfur atom. Representative examples of cycloalkenylthio include, but are not limited to, cyclopropenylthio, cyclobutenylthio, cyclopentenylthio, cyclohexenylthio, cycloheptenylthio, and cyclooctenylthio.

The term “cycloalkenylthioalkyl” as used herein, means a cycloalkenylthio group, as defined herein, appended to the parent molecular moiety through an alkyl group, as defined herein. Representative examples of cycloalkenylthioalkyl include, but are not limited to, cyclopropenylthiomethyl, cyclobutenylthiomethyl, cyclopentenylthiomethyl, cyclohexenylthiomethyl, cycloheptenylthiomethyl, and cyclooctenylthiomethyl.

The term “cycloalkyl” as used herein, means a saturated cyclic hydrocarbon group containing from 3 to 8 carbons, examples of cycloalkyl include cyclopropyl, cyclobutyl, cyclopentyl, cyclohexyl, cycloheptyl, and cyclooctyl.

The cycloalkyl groups of this invention can be substituted with 0, 1, 2, 3, or 4 substituents independently a member selected from the group consisting of alkenyl, alkoxy, alkoxyalkyl, alkoxycarbonyl, alkoxycarbonylalkyl, alkyl, alkylcarbonyl, alkylcarbonylalkyl, alkylcarbonyloxy, alkylsulfonyl, alkylthio, alkynyl, carboxy, carboxyalkyl, cyano, cyanoalkyl, formyl, halogen, haloalkyl, hydroxy, hydroxyalkyl, mercapto, —NZ₅Z₆ and (NZ₅Z₆)alkyl.

The term “cycloalkylalkoxy” as used herein, means a cycloalkyl group, as defined herein, appended to the parent molecular moiety through an alkoxy group, as defined herein. Representative examples of cycloalkylalkoxy include, but are not limited to, cyclopropylmethoxy, cyclobutylmethoxy, cyclopentylmethoxy, cyclohexylmethoxy, 2-cyclohexylethoxy, cycloheptylmethoxy, and cyclooctylmethoxy.

The term “cycloalkylalkoxyalkyl” as used herein, means a cycloalkylalkoxy group, as defined herein appended to the parent molecular moiety through an alkyl group, as defined herein. Representative examples of cycloalkoxyalkyl include, but are not limited to, cyclopropylmethoxymethyl, cyclobutylmethoxymethyl, cyclopentylmethoxymethyl, cyclohexylmethoxymethyl, (2-cyclohexylethoxy)methyl, cycloheptylmethoxymethyl, and cyclooctylmethoxymethyl.

The term “cycloalkylalkyl” as used herein, means a cycloalkyl group, as defined herein, appended to the parent molecular moiety through an alkyl group as defined herein. Representative examples of cycloalkylalkyl include, but are not limited to, cyclopropylmethyl, cyclobutylmethyl, cyclopentylmethyl, cyclohexylmethyl, 2-cyclohexylethyl, cycloheptylmethyl, and cyclooctylmethyl.

The term “cycloalkylalkylthio” as used herein, means a cycloalkylalkyl group, as defined herein, appended to the parent molecular moiety through a sulfur atom. Representative examples of cycloalkylalkylthio include, but are not limited to, cyclopropylmethylthio, cyclobutylmethylthio, cyclopentylmethylthio, cyclohexylmethylthio, 2-cyclohexylethylthio, cycloheptylmethylthio, and cyclooctylmethylthio.

The term “cycloalkylalkylthioalkyl” as used herein, means a cycloalkylalkylthio group, as defined herein, appended to the parent molecular moiety through an alkyl group, as defined herein. Representative examples of cycloalkylalkylthioalkyl include, but are not limited to, cyclopropylmethylthiomethyl, cyclobutylmethylthiomethyl, cyclopentylmethylthiomethyl, cyclohexylmethylthiomethyl, 2-cyclohexylethylthiomethyl, cycloheptylmethylthiomethyl, and cyclooctylmethylthiomethyl.

The term “cycloalkylcarbonyl” as used herein, means a cycloalkyl group, as defined herein, appended to the parent molecular moiety through a carbonyl group as defined herein. Representative examples of cycloalkylcarbonyl include, but are not limited to, cyclopropylcarbonyl, cyclobutylcarbonyl, cyclopentylcarbonyl, cyclohexylcarbonyl, cycloheptylcarbonyl, and cyclooctylcarbonyl.

The term “cycloalkyloxy” as used herein, means a cycloalkyl group, as defined herein, appended to the parent molecular moiety through an oxygen atom, examples of cycloalkyloxy include cyclopropyloxy, cyclobutyloxy, cyclopentyloxy, cyclohexyloxy, cycloheptyloxy, and cyclooctyloxy.

The term “cycloalkyloxyalkyl” as used herein, means a cycloalkyloxy group, as defined herein, appended to the parent molecular moiety through an alkyl group, as defined herein. Representative examples of cycloalkyloxyalkyl include, but are not limited to, cyclopropyloxymethyl, cyclobutyloxymethyl, cyclopentyloxymethyl, cyclohexyloxymethyl, cycloheptyloxymethyl, and cyclooctyloxymethyl.

The term “cycloalkylthio” as used herein, means a cycloalkyl group, as defined herein, appended to the parent molecular moiety through a sulfur atom, examples of cycloalkylthio include cyclopropylthio, cyclobutylthio, cyclopentylthio, cyclohexylthio, cycloheptylthio, and cyclooctylthio.

The term “cycloalkylthioalkyl” as used herein, means a cycloalkylthio group, as defined herein, appended to the parent molecular moiety through an alkyl group, as defined herein. Representative examples of cycloalkylthioalkyl include cyclopropylthiomethyl, cyclobutylthiomethyl, cyclopentylthiomethyl, cyclohexylthiomethyl, cycloheptylthiomethyl, and cyclooctylthiomethyl.

The term “formyl,” as used herein, means a —C(O)H group.

The term “formylalkyl” as used herein, means a formyl group, as defined herein, appended to the parent molecular moiety through an alkyl group as defined herein. Representative examples of formylalkyl include, but are not limited to, cyclopropylmethyl, cyclobutylmethyl, cyclopentylmethyl, cyclohexylmethyl, 2-oxoethyl, 3-oxopropyl, and 4-oxobutyl.

The term “halo” or “halogen,” as used herein, means —Cl, —Br, —I or —F.

The term “haloalkoxy,” as used herein, means at least one halogen, as defined herein, appended to the parent molecular moiety through an alkoxy group, as defined herein. Representative examples of haloalkoxy include, but are not limited to, chloromethoxy, 2-fluoroethoxy, trifluoromethoxy, pentafluoroethoxy, and 2-chloro-3-fluoropentoxy.

The term “haloalkyl,” as used herein, means at least one halogen, as defined herein, appended to the parent molecular moiety through an alkyl group, as defined herein. Representative examples of haloalkyl include, but are not limited to, chloromethyl, 2-fluoroethyl, trifluoromethyl, pentafluoroethyl, and 2-chloro-3-fluoropentyl.

The term “haloalkylcarbonyl,” as used herein, means a haloalkyl group, as defined herein, appended to the parent molecular moiety through a carbonyl group, as defined herein. Representative examples of haloalkylcarbonyl include, but are not limited to, chloromethylcarbonyl, 2-fluoroethylcarbonyl, trifluoromethylcarbonyl, pentafluoroethylcarbonyl, and 2-chloro-3-fluoropentylcarbonyl.

The term “haloalkylsulfonyl,” as used herein, means a haloalkyl group, as defined herein, appended to the parent molecular moiety through a sulfonyl group, as defined herein. Representative examples of haloalkylsulfonyl include, but are not limited to, chloromethylsulfonyl, 2-fluoroethylsulfonyl, trifluoromethylsulfonyl, pentafluoroethylsulfonyl, and 2-chloro-3-fluoropentylsulfonyl.

The term “heteroaryl,” as used herein, means an aromatic monocyclic ring or an aromatic bicyclic ring. The aromatic monocyclic rings are five or six membered rings wherein 1, 2, 3, or 4 atoms are independently a member selected from the group consisting of N, O, and S. The five membered aromatic monocyclic rings have two double bonds and the six membered aromatic monocyclic rings have three double bonds. The aromatic bicyclic rings are composed of an aromatic monocyclic ring fused to a phenyl group, alternatively, an aromatic monocyclic ring is fused to another aromatic monocyclic ring. The aromatic monocyclic rings and the aromatic bicyclic rings are connected to the parent molecular moiety through a carbon or nitrogen atom. Representative examples of heteroaryl include, but are not limited to, benzimidazole, benzothienyl, benzoxadiazolyl, cinnolinyl, dibenzofuranyl, furopyridinyl, furyl, imidazolyl, indazolyl, indolyl, isoxazolyl, isoquinolinyl, isothiazolyl, naphthyridinyl, oxadiazolyl, oxazolyl, pyridinyl, pyridazinyl, pyrimidinyl, pyrazinyl, pyrazolyl, pyrrolyl, quinolinyl, tetrazolyl, thiadiazolyl, thiazolyl, thienopyridinyl, thienyl, triazolyl, and triazinyl.

The heteroaryl groups of the present invention are substituted with 0, 1, 2, 3, or 4 substituents independently a member selected from the group consisting of alkenyl, alkoxy, alkoxyalkyl, alkoxycarbonyl, alkoxycarbonylalkyl, alkyl, alkylcarbonyl, alkylcarbonylalkyl, alkylcarbonyloxy, alkylsulfonyl, alkylthio, alkynyl, arylcarbonyl, arylsulfonyl, carboxy, carboxyalkyl, cyano, cyanoalkyl, formyl, haloalkoxy, haloalkyl, haloalkylcarbonyl, haloalkylsulfonyl, halogen, hydroxy, hydroxyalkyl, hydroxyhaloalkyl, mercapto, nitro, —NZ₅Z₆ and (NZ₅Z₆)alkyl.

The term “heteroarylalkoxy” as used herein, means a heteroaryl group, as defined herein, appended to the parent molecular moiety through an alkoxy group, as defined herein. Representative examples of heteroarylalkoxy include, but are not limited to, fur-3-ylmethoxy, 1H-imidazol-2-ylmethoxy, 1H-imidazol-4-ylmethoxy, 1-(pyridin-4-yl)ethoxy, pyridin-3-ylmethoxy, 6-chloropyridin-3-ylmethoxy, pyridin-4-ylmethoxy, (6-(trifluoromethyl)pyridin-3-yl)methoxy, (6-(cyano)pyridin-3-yl)methoxy, (2-(cyano)pyridin-4-yl)methoxy, (5-(cyano)pyridin-2-yl)methoxy, (2-(chloro)pyridin-4-yl)methoxy, pyrimidin-5-ylmethoxy, 2-(pyrimidin-2-yl)propoxy, thien-2-ylmethoxy, and thien-3-ylmethoxy.

The term “heteroarylalkoxyalkyl” as used herein, means a heteroarylalkoxy group, as defined herein, appended to the parent molecular moiety through an alkyl group, as defined herein. Representative examples of heteroarylalkoxyalkyl include, but are not limited to, fur-3-ylmethoxymethyl, 1H-imidazol-2-ylmethoxymethyl, 1H-imidazol-4-ylmethoxymethyl, pyridin-3-ylmethoxymethyl, 6-chloropyridin-3-ylmethoxymethyl, pyridin-4-ylmethoxymethyl, (6-(trifluoromethyl)pyridin-3-yl)methoxymethyl, (6-(cyano)pyridin-3-yl)methoxymethyl, (2-(cyano)pyridin-4-yl)methoxymethyl, (5-(cyano)pyridin-2-yl)methoxymethyl, (2-(chloro)pyridin-4-yl)methoxymethyl, pyrimidin-5-ylmethoxymethyl, 2-(pyrimidin-2-yl)propoxymethyl, thien-2-ylmethoxymethyl, and thien-3-ylmethoxymethyl.

The term “heteroarylalkyl” as used herein, means a heteroaryl, as defined herein, appended to the parent molecular moiety through an alkyl group, as defined herein. Representative examples of heteroarylalkyl include, but are not limited to, fur-3-ylmethyl, 1H-imidazol-2-ylmethyl, 1H-imidazol-4-ylmethyl, 1-(pyridin-4-yl)ethyl, pyridin-3-ylmethyl, 6-chloropyridin-3-ylmethyl, pyridin-4-ylmethyl, (6-(trifluoromethyl)pyridin-3-yl)methyl, (6-(cyano)pyridin-3-yl)methyl, (2-(cyano)pyridin-4-yl)methyl, (5-(cyano)pyridin-2-yl)methyl, (2-(chloro)pyridin-4-yl)methyl, pyrimidin-5-ylmethyl, 2-(pyrimidin-2-yl)propyl, thien-2-ylmethyl, and thien-3-ylmethyl.

The term “heteroarylalkylthio” as used herein, means a heteroarylalkyl group, as defined herein, appended to the parent molecular moiety through a sulfur atom. Representative examples of heteroarylalkylthio include, but are not limited to, fur-3-ylmethylthio, 1H-imidazol-2-ylmethylthio, 1H-imidazol-4-ylmethylthio, pyridin-3-ylmethylthio, 6-chloropyridin-3-ylmethylthio, pyridin-4-ylmethylthio, (6-(trifluoromethyl)pyridin-3-yl)methylthio, (6-(cyano)pyridin-3-yl)methylthio, (2-(cyano)pyridin-4-yl)methylthio, (5-(cyano)pyridin-2-yl)methylthio, (2-(chloro)pyridin-4-yl)methylthio, pyrimidin-5-ylmethylthio, 2-(pyrimidin-2-yl)propylthio, thien-2-ylmethylthio, and thien-3-ylmethylthio.

The term “heteroarylalkylthioalkyl” as used herein, means a heteroarylalkylthio group, as defined herein, appended to the parent molecular moiety through an alkyl group, as defined herein. Representative examples of heteroarylalkylthioalkyl include, but are not limited to, fur-3-ylmethylthiomethyl, 1H-imidazol-2-ylmethylthiomethyl, 1H-imidazol-4-ylmethylthiomethyl, pyridin-3-ylmethylthiomethyl, 6-chloropyridin-3-ylmethylthiomethyl, pyridin-4-ylmethylthiomethyl, (6-(trifluoromethyl)pyridin-3-yl)methylthiomethyl, (6-(cyano)pyridin-3-yl)methylthiomethyl, (2-(cyano)pyridin-4-yl)methylthiomethyl, (5-(cyano)pyridin-2-yl)methylthiomethyl, (2-(chloro)pyridin-4-yl)methylthiomethyl, pyrimidin-5-ylmethylthiomethyl, 2-(pyrimidin-2-yl)propylthiomethyl, thien-2-ylmethylthiomethyl, and thien-3-ylmethylthiomethyl.

The term “heteroarylcarbonyl” as used herein, means a heteroaryl group, as defined herein, appended to the parent molecular moiety through a carbonyl group, as defined herein. Representative examples of heteroarylcarbonyl include, but are not limited to, fur-3-ylcarbonyl, 1H-imidazol-2-ylcarbonyl, 1H-imidazol-4-ylcarbonyl, pyridin-3-ylcarbonyl, 6-chloropyridin-3-ylcarbonyl, pyridin-4-ylcarbonyl, (6-(trifluoromethyl)pyridin-3-yl)carbonyl, (6-(cyano)pyridin-3-yl)carbonyl, (2-(cyano)pyridin-4-yl)carbonyl, (5-(cyano)pyridin-2-yl)carbonyl, (2-(chloro)pyridin-4-yl)carbonyl, pyrimidin-5-ylcarbonyl, pyrimidin-2-ylcarbonyl, thien-2-ylcarbonyl, and thien-3-ylcarbonyl.

The term “heteroaryloxy” as used herein, means a heteroaryl group, as defined herein, appended to the parent molecular moiety through an oxygen atom. Representative examples of heteroaryloxy include, but are not limited to, fur-3-yloxy, 1H-imidazol-2-yloxy, 1H-imidazol-4-yloxy, pyridin-3-yloxy, 6-chloropyridin-3-yloxy, pyridin-4-yloxy, (6-(trifluoromethyl)pyridin-3-yl)oxy, (6-(cyano)pyridin-3-yl)oxy, (2-(cyano)pyridin-4-yl)oxy, (5-(cyano)pyridin-2-yl)oxy, (2-(chloro)pyridin-4-yl)oxy, pyrimidin-5-yloxy, pyrimidin-2-yloxy, thien-2-yloxy, and thien-3-yloxy.

The term “heteroaryloxyalkyl” as used herein, means a heteroaryloxy group, as defined herein, appended to the parent molecular moiety through an alkyl group, as defined herein. Representative examples of heteroaryloxyalkyl include, but are not limited to, fur-3-yloxymethyl, 1H-imidazol-2-yloxymethyl, 1H-imidazol-4-yloxymethyl, pyridin-3-yloxymethyl, 6-chloropyridin-3-yloxymethyl, pyridin-4-yloxymethyl, (6-(trifluoromethyl)pyridin-3-yl)oxymethyl, (6-(cyano)pyridin-3-yl)oxymethyl, (2-(cyano)pyridin-4-yl)oxymethyl, (5-(cyano)pyridin-2-yl)oxymethyl, (2-(chloro)pyridin-4-yl)oxymethyl, pyrimidin-5-yloxymethyl, pyrimidin-2-yloxymethyl, thien-2-yloxymethyl, and thien-3-yloxymethyl.

The term “heteroarylthio” as used herein, means a heteroaryl group, as defined herein, appended to the parent molecular moiety through a sulfur atom. Representative examples of heteroarylthio include, but are not limited to, fur-3-ylthio, 1H-imidazol-2-ylthio, 1H-imidazol-4-ylthio, pyridin-3-ylthio, 6-chloropyridin-3-ylthio, pyridin4-ylthio, (6-(trifluoromethyl)pyridin-3-yl)thio, (6-(cyano)pyridin-3-yl)thio, (2-(cyano)pyridin-4-yl)thio, (5-(cyano)pyridin-2-yl)thio, (2-(chloro)pyridin-4-yl)thio, pyrimidin-5-ylthio, pyrimidin-2-ylthio, thien-2-ylthio, and thien-3-ylthio.

The term “heteroarylthioalkyl” as used herein, means a heteroarylthio group, as defined herein, appended to the parent molecular moiety through an alkyl group, as defined herein. Representative examples of heteroarylthioalkyl include, but are not limited to, fur-3-ylthiomethyl, 1H-imidazol-2-ylthiomethyl, 1H-imidazol-4-ylthiomethyl, pyridin-3-ylthiomethyl, 6-chloropyridin-3-ylthiomethyl, pyridin-4-ylthiomethyl, (6-(trifluoromethyl)pyridin-3-yl)thiomethyl, (6-(cyano)pyridin-3-yl)thiomethyl, (2-(cyano)pyridin-4-yl)thiomethyl, (5-(cyano)pyridin-2-yl)thiomethyl, (2-(chloro)pyridin-4-yl)thiomethyl, pyrimidin-5-ylthiomethyl, pyrimidin-2-ylthiomethyl, thien-2-ylthiomethyl, and thien-3-ylthiomethyl.

The term “heterocycle,” as used herein, means a non-aromatic monocyclic ring or a non-aromatic bicyclic ring. The non-aromatic monocyclic ring is a three, four, five, six, seven, or eight membered ring containing 1 or 2 heteroatoms independently a member selected from the group consisting of N, O, and S. The three membered rings have zero double bonds. The four and five membered rings have zero or one double bond. The six membered rings have zero, one, or two double bonds. The seven and eight membered rings have zero, one, two, or three double bonds. The bicyclic non-aromatic rings are composed of a non-aromatic monocyclic ring fused to a phenyl group. Alternatively, bicyclic non-aromatic rings are composed of a non-aromatic monocyclic ring fused to another non-aromatic monocyclic ring. The heterocycle groups of the present invention can be attached to the parent molecular moiety through a carbon atom or a nitrogen atom. Representative examples of heterocycle include, but are not limited to, azetidinyl, 1,3-benzodioxolyl, 1,3-benzodioxol-4-yl, hexahydro-1H-azepinyl, hexahydroazocin-(2H)-yl, morpholinyl, piperazinyl, piperidinyl, pyrrolidinyl, tetrahydrofuranyl, tetrahydrofuran-2-yl, tetrahydrofuran-3-yl, tetrahydro-2H-pyranyl, tetrahydro-2H-pyran-2-yl, tetrahydro-2H-pyran-4-yl, tetrahydrothienyl, tetrahydrothien-2-yl, and tetrahydrothien-3-yl, and thiomorpholinyl.

The heterocycles of the present invention are substituted with 0, 1, 2, 3, or 4 substituents independently a member selected from alkenyl, alkoxy, alkoxyalkyl, alkoxycarbonyl, alkoxycarbonylalkyl, alkyl, alkylcarbonyl, alkylcarbonylalkyl, alkylcarbonyloxy, alkylsulfonyl, alkylthio, alkynyl, carboxy, carboxyalkyl, cyano, cyanoalkyl, formyl, haloalkoxy, haloalkyl, halogen, hydroxy, hydroxyalkyl, mercapto, nitro, —NZ₅Z₆ and (NZ₅Z₆)alkyl.

The term “heterocyclealkoxy” as used herein, means a heterocycle group, as defined herein, appended to the parent molecular moiety through an alkoxy group, as defined herein. Representative examples of heterocyclealkoxy include, but are not limited to, 1,3-benzodioxol-4-ylmethoxy, pyridin-3-ylmethoxy, 2-pyrimidin-2-ylpropoxy, tetrahydrofuran-2-ylmethoxy, tetrahydrofuran-3-ylmethoxy, tetrahydro-2H-pyran-2-ylmethoxy, tetrahydro-2H-pyran-4-ylmethoxy, tetrahydrothien-2-ylmethoxy, and tetrahydrothien-3-ylmethoxy.

The term “heterocyclealkoxyalkyl” as used herein, means a heterocyclealkoxy group, as defined herein, appended to the parent molecular moiety through an alkyl group, as defined herein. Representative examples of heterocyclealkoxyalkyl include, but are not limited to, 1,3-benzodioxol-4-ylmethoxymethyl, pyridin-3-ylmethoxymethyl, 2-pyrimidin-2-ylpropoxymethyl, tetrahydrofuran-2-ylmethoxymethyl, tetrahydrofuran-3-ylmethoxymethyl, tetrahydro-2H-pyran-2-ylmethoxymethyl, tetrahydro-2H-pyran-4-ylmethoxymethyl, tetrahydrothien-2-ylmethoxymethyl, and tetrahydrothien-3-ylmethoxymethyl.

The term “heterocyclealkyl” as used herein, means a heterocycle group, as defined herein, appended to the parent molecular moiety through an alkyl group, as defined herein. Representative examples of heterocyclealkyl include, but are not limited to, 1,3-benzodioxol-4-ylmethyl, pyridin-3-ylmethyl, 2-pyrimidin-2-ylpropyl, tetrahydrofuran-2-ylmethyl, tetrahydrofuran-3-ylmethyl, tetrahydro-2H-pyran-2-ylmethyl, tetrahydro-2H-pyran-4-ylmethyl, tetrahydrothien-2-ylmethyl, and tetrahydrothien-3-ylmethyl.

The term “heterocyclealkylthio” as used herein, means a heterocyclealkyl group, as defined herein, appended to the parent molecular moiety through a sulfur atom. Representative examples of heterocyclealkylthio include, but are not limited to, 1,3-benzodioxol-4-ylmethylthio, pyridin-3-ylmethylthio, 2-pyrimidin-2-ylpropylthio, tetrahydrofuran-2-ylmethylthio, tetrahydrofuran-3-ylmethylthio, tetrahydro-2H-pyran-2-ylmethylthio, tetrahydro-2H-pyran-4-ylmethylthio, tetrahydrothien-2-ylmethylthio, and tetrahydrothien-3-ylmethylthio.

The term “heterocyclealkylthioalkyl” as used herein, means a heterocyclealkylthio group, as defined herein, appended to the parent molecular moiety through an alkyl group, as defined herein. Representative examples of heterocyclealkylthioalkyl include, but are not limited to, 1,3-benzodioxol-4-ylmethylthiomethyl, pyridin-3-ylmethylthiomethyl, 2-pyrimidin-2-ylpropylthiomethyl, tetrahydrofuran-2-ylmethylthiomethyl, tetrahydrofuran-3-ylmethylthiomethyl, tetrahydro-2H-pyran-2-ylmethylthiomethyl, tetrahydro-2H-pyran-4-ylmethylthiomethyl, tetrahydrothien-2-ylmethylthiomethyl, and tetrahydrothien-3-ylmethylthiomethyl.

The term “heterocyclecarbonyl” as used herein, means a heterocycle group, as defined herein, appended to the parent molecular moiety through a carbonyl group, as defined herein. Representative examples of heterocyclecarbonyl include, but are not limited to, 1,3-benzodioxol-4-ylcarbonyl, pyridin-3-ylcarbonyl, pyrimidin-2-ylcarbonyl, tetrahydrofuran-2-ylcarbonyl, tetrahydrofuran-3-ylcarbonyl, tetrahydro-2H-pyran-2-ylcarbonyl, tetrahydro-2H-pyran-4-ylcarbonyl, tetrahydrothien-2-ylcarbonyl, and tetrahydrothien-3-ylcarbonyl.

The term “heterocycleoxy” as used herein, means a heterocycle group, as defined herein, appended to the parent molecular moiety through an oxygen atom. Representative examples of heterocycleoxy include, but are not limited to, 1,3-benzodioxol-4-yloxy, pyridin-3-yloxy, 2-pyrimidin-2-yloxy, tetrahydrofuran-2-yloxy, tetrahydrofuran-3-yloxy, tetrahydro-2H-pyran-2-yloxy, tetrahydro-2H-pyran-4-yloxy, tetrahydrothien-2-yloxy, and tetrahydrothien-3-yloxy.

The term “heterocycleoxyalkyl” as used herein, means a heterocycleoxy group, as defined herein, appended to the parent molecular moiety through an alkyl group, as defined herein. Representative examples of heterocycleoxyalkyl include, but are not limited to, 1,3-benzodioxol-4-yloxymethyl, pyridin-3-yloxymethyl, 2-pyrimidin-2-yloxymethyl, tetrahydrofuran-2-yloxymethyl, tetrahydrofuran-3-yloxymethyl, tetrahydro-2H-pyran-2-yloxymethyl, tetrahydro-2H-pyran-4-yloxymethyl, tetrahydrothien-2-yloxymethyl, and tetrahydrothien-3-yloxymethyl.

The term “heterocyclethio” as used herein, means a heterocycle group, as defined herein, appended to the parent molecular moiety through a sulfur atom. Representative examples of heterocyclethio include, but are not limited to, 1,3-benzodioxol-4-ylthio, pyridin-3-ylthio, 2-pyrimidin-2-ylthio, tetrahydrofuran-2-ylthio, tetrahydrofuran-3-ylthio, tetrahydro-2H-pyran-2-ylthio, tetrahydro-2H-pyran-4-ylthio, tetrahydrothien-2-ylthio, and tetrahydrothien-3-ylthio.

The term “heterocyclethioalkyl” as used herein, means a heterocyclethio group, as defined herein, appended to the parent molecular moiety through an alkyl group, as defined herein. Representative examples of heterocyclethioalkyl include, but are not limited to, 1,3-benzodioxol-4-ylthiomethyl, pyridin-3-ylthiomethyl, 2-pyrimidin-2-ylthiomethyl, tetrahydrofuran-2-ylthiomethyl, tetrahydrofuran-3-ylthiomethyl, tetrahydro-2H-pyran-2-ylthiomethyl, tetrahydro-2H-pyran-4-ylthiomethyl, tetrahydrothien-2-ylthiomethyl, and tetrahydrothien-3-ylthiomethyl.

The term “hydroxy” as used herein, means an —OH group.

The term “hydroxyalkyl” as used herein, means at least one hydroxy group, as defined herein, appended to the parent molecular moiety through an alkyl group, as defined herein. Representative examples of hydroxyalkyl include, but are not limited to, 2-hydroxyethyl, 2-hydroxypropyl, 1,2-dihydroxypropyl, 3-hydroxybutyl and the like.

The term “hydroxyhaloalkyl” as used herein, means at least one hydroxy group, as defined herein, appended to the parent molecular moiety through a haloalkyl group, as defined herein.

The term “—NR_{A}R_{B}” as used herein, means two groups, R_{A} and R_{B}, which are appended to the parent molecular moiety through a nitrogen atom. R_{A} and R_{B} are each independently a member selected from the group consisting of hydrogen, alkoxycarbonyl, alkyl, alkylcarbonyl, alkoxysulfonyl, alkylsulfonyl, aryl, arylalkyl, and formyl. Representative examples of —NR_{A}R_{B} include, but are not limited to, amino, methylamino, acetylamino, and acetylmethylamino.

The term “—NR_{C}R_{D}” as used herein, means two groups, R_{C} and R_{D}, which are appended to the parent molecular moiety through a nitrogen atom. R_{C} and R_{D} are each independently a member selected from the group consisting of hydrogen, alkoxycarbonyl, alkenyl, alkyl, alkylcarbonyl, alkoxysulfonyl, alkylsulfonyl, aryl, arylalkyl, cycloalkyl, cycloalkylalkyl, formyl, and hydroxyalkyl. Representative examples of —NR_{C}R_{D} include, but are not limited to, amino, methylamino, acetylamino, acetylmethylamino, benzylamino, benzyl(methyl)amino, dimethylamino, methylamino, ethylamino, diethylamino, cyclohexylamino, cyclohexylmethylamino, and phenylamino.

The term “(NR_{C}R_{D})alkyl” as used herein, means a —NR_{C}R_{D} group, as defined herein, appended to the parent molecular moiety through an alkyl group, as defined herein. Representative examples of (NR_{C}R_{D})alkyl include, but are not limited to, aminomethyl, methylaminomethyl, acetylaminomethyl, acetylmethylaminomethyl, benzylaminomethyl, benzyl(methyl)amino, dimethylaminomethyl, ethylaminomethyl, diethylaminomethyl, cyclohexylaminomethyl, cyclohexylmethylaminomethyl, butylaminomethyl, 3-methylphenylaminomethyl, and phenylaminomethyl.

The term “—NR_{E}R_{F}” as used herein, means two groups, R_{E} and R_{F}, which are appended to the parent molecular moiety through a nitrogen atom. R_{E} and R_{F} are each independently a member selected from the group consisting of hydrogen, alkoxyalkyl, alkoxyalkylcarbonyl, alkoxycarbonyl, alkoxysulfonyl, alkyl, alkylcarbonyl, alkylsulfonyl, alkylthioalkyl, alkylthioalkylcarbonyl, alkylthiocarbonyl, aryl, arylalkoxyalkyl, arylalkyl, arylcarbonyl, cycloalkyl, cycloalkylalkyl, cycloalkylcarbonyl, formyl, heteroaryl, heteroarylalkyl, heteroarylcarbonyl, heterocycle, heterocyclealkyl, heterocyclecarbonyl, (NZ₁Z₂)alkyl, and (NZ₁Z₂)carbonyl. Representative examples of —NR_{E}R_{F} include, but are not limited to, amino, methylamino, acetylamino, acetylmethylamino, benzylamino, butylamino, 3-methylphenylamino, and phenylamino.

The term “(NR_{E}R_{F})alkyl” as used herein, means a —NR_{E}R_{F} group, as defined herein, appended to the parent molecular moiety through an alkyl group, as defined herein. Representative examples of (NR_{E}R_{F})alkyl include, but are not limited to, aminomethyl, methylaminomethyl, acetylaminomethyl, acetylmethylaminomethyl, benzylaminomethyl, butylaminomethyl, 3-methylphenylaminomethyl, and phenylaminomethyl.

The term “(NR_{E}R_{F})carbonyl” as used herein, means a —NR_{E}R_{F} group, as defined herein, appended to the parent molecular moiety through a carbonyl group, as defined herein. Representative examples of (NR_{E}R_{F})carbonyl include, but are not limited to, aminocarbonyl, methylaminocarbonyl, acetylaminocarbonyl, acetylmethylaminocarbonyl, benzylaminocarbonyl, butylaminocarbonyl, 3-methylphenylaminocarbonyl, and phenylaminocarbonyl.

The term “(NR_{E}R_{F})carbonylalkenyl” as used herein, means a (NR_{E}R_{F})carbonyl group, as defined herein, appended to the parent molecular moiety through an alkenyl group, as defined herein.

The term “(NR_{E}R_{F})carbonylalkyl” as used herein, means a (NR_{E}R_{F})carbonyl group, as defined herein, appended to the parent molecular moiety through an alkyl group, as defined herein. Representative examples of (NR_{E}R_{F})carbonylalkyl include, but are not limited to, aminocarbonylmethyl, methylaminocarbonylmethyl, acetylaminocarbonylmethyl, acetylmethylaminocarbonylmethyl, 2-(benzylaminocarbonyl)ethyl, 2-(butylaminocarbonyl)ethyl, 2-(3-methylphenylaminocarbonyl)ethyl, and 2-(phenylaminocarbonyl)ethyl.

The term “(NR_{E}R_{F})sulfonyl” as used herein, means a —NR_{E}R_{F} group, as defined herein, appended to the parent molecular moiety through a sulfonyl group, as defined herein. Representative examples of (NR_{E}R_{F})sulfonyl include, but are not limited to, aminosulfonyl and dimethylaminosulfonyl.

The term “(NR_{E}R_{F})sulfonylalkyl” as used herein, means a (NR_{E}R_{F})sulfonyl group, as defined herein, appended to the parent molecular moiety through an alkyl group, as defined herein. Representative examples of (NR_{E}R_{F})sulfonylalkyl include, but are not limited to, aminosulfonylmethyl and dimethylaminosulfonylmethyl.

The term “—NR_{G}R_{H}” as used herein, means two groups, R_{G} and R_{H}, which are appended to the parent molecular moiety through a nitrogen atom. R_{G} and R_{H} are each independently a member selected from the group consisting of hydrogen, alkoxyalkyl, alkoxyalkylcarbonyl, alkoxycarbonyl, alkoxysulfonyl, alkyl, alkylcarbonyl, alkylsulfonyl, alkylthioalkyl, alkylthioalkylcarbonyl, alkylthiocarbonyl, aryl, arylalkoxyalkyl, arylalkyl, arylcarbonyl, cycloalkyl, cycloalkylalkyl, cycloalkylcarbonyl, formyl, heteroaryl, heteroarylalkyl, heteroarylcarbonyl, heterocycle, heterocyclealkyl, heterocyclecarbonyl, (NZ₃Z₄)alkyl, and (NZ₃Z₄)carbonyl. Representative examples of —NR_{G}R_{H} include, but are not limited to, amino, methylamino, acetylamino, acetylmethylamino, benzylamino, (2-(benzyloxy)ethyl)amino, butylamino, cyclohexylmethylamino, cycloheptylamino, dimethylamino, ethylamino, (1-ethylpropyl)amino, isobutylamino, 3-methylphenylamino, neopentylamino, 4-nitrobenzylamino, 4-nitrophenylamino, (2-(4-nitrophenyl)ethyl)amino, phenylamino, propylamino, propylaminocarbonylamino, propionylamino, (1,3-benzodioxol-4-ylmethyl)amino, (butoxyacetyl)amino, 4-chlorobenzylamino, (4-chlorobenzyl)acetylamino, (4-chlorobenzyl)formylamino, (4-chlorobenzyl)methylamino, (1-(4-chlorophenyl)ethyl)amino, (2-(4-chlorophenyl)ethyl)amino, 2-chloropyridin-4-ylmethylamino, 6-chloropyridin-3-ylmethylamino, cyclopropylmethylamino, 3,4-dichlorobenzylamino, 4-cyanobenzylamino, (4-cyanobenzyl)methylamino, 4-cyanophenylamino, (1-(4-cyanophenyl)ethyl)amino, 2-(cyano)pyridin-4-ylmethylamino, 5-(cyano)pyridin-2-ylmethylamino, 6-(cyano)pyridin-3-ylmethylamino, (2-(tert-butoxycarbonylamino)ethyl)amino, fur-3-ylmethylamino, 4-methoxybenzylamino, tetrahydrofuran-3-ylmethylamino, tetrahydro-2H-pyran-4-ylmethylamino, (4-chlorophenylcarbonyl)amino, pyridin-2-ylmethylamino, pyridin-3-ylmethylamino, pyridin-4-ylmethylamino, (1-(pyridin-4-yl)ethyl)amino, pyrimidin-5-ylmethylamino, 1H-imidazol-4-ylmethylamino, 1H-imidazol-2-ylmethylamino, thien-2-ylmethylamino, thien-3-ylmethylamino, 4-(trifluoromethoxy)benzylamino, and 6-(trifluoromethyl)pyridin-3-ylmethylamino.

The term “(NR_{G}R_{H})alkyl” as used herein, means a —NR_{G}R_{H} group, as defined herein, appended to the parent molecular moiety through an alkyl group, as defined herein. Representative examples of (NR_{G}R_{H})alkyl include, but are not limited to, aminomethyl, methylaninomethyl, acetylaminomethyl, acetylmethylaminomethyl, benzylaminomethyl, (2-(benzyloxy)ethyl)aminomethyl, butylaminomethyl, cyclohexylmethylaminomethyl, cycloheptylaminomethyl, dimethylaminomethyl, ethylaminomethyl, (1-ethylpropyl)aminomethyl, isobutylaminomethyl, 3-methylphenylaminomethyl, neopentylaminomethyl, 4-nitrobenzylaminomethyl, 4-nitrophenylaminomethyl, (2-(4-nitrophenyl)ethyl)aminomethyl, phenylaminomethyl, propylaminomethyl, propylaminocarbonylaminomethyl, propionylaminomethyl, (1,3-benzodioxol-4-ylmethyl)aminomethyl, (butoxyacetyl)aminomethyl, 4-chlorobenzylaminomethyl, (4-chlorobenzyl)acetylaminomethyl, (4-chlorobenzyl)formylaminomethyl, (4-chlorobenzyl)methylaminomethyl, (1-(4-chlorophenyl)ethyl)aminomethyl, (2-(4-chlorophenyl)ethyl)aminomethyl, 2-chloropyridin-4-ylmethylaminomethyl, 6-chloropyridin-3-ylmethylaminomethyl, cyclopropylmethylaminomethyl, 3,4-dichlorobenzylaminomethyl, 4-cyanobenzylaminomethyl, (4-cyanobenzyl)methylaminomethyl, 4-cyanophenylaminomethyl, (1-(4-cyanophenyl)ethyl)aminomethyl, 2-(cyano)pyridin-4-ylmethylaminomethyl, 5-(cyano)pyridin-2-ylmethylaminomethyl, 6-(cyano)pyridin-3-ylmethylaminomethyl, (2-(tert-butoxycarbonylamino)ethyl)aminomethyl, fur-3-ylmethylaminomethyl, 4-methoxybenzylaminomethyl, tetrahydrofuran-3-ylmethylaminomethyl, tetrahydro-2H-pyran-4-ylmethylaminomethyl, (4-chlorophenylcarbonyl)aminomethyl, pyridin-2-ylmethylaminomethyl, pyridin-3-ylmethylaminomethyl, pyridin-4-ylmethylaminomethyl, (1-(pyridin-4-yl)ethyl)aminomethyl, pyrimidin-5-ylmethylaminomethyl, 1H-imidazol-4-ylmethylaminomethyl, 1H-imidazol-2-ylmethylaminomethyl, thien-2-ylmethylaminomethyl, thien-3-ylmethylaminomethyl, 4-(trifluoromethoxy)benzylaminomethyl, and 6-(trifluoromethyl)pyridin-3-ylmethylaminomethyl.

The term “(NR_{G}R_{H})carbonyl” as used herein, means a —NR_{G}R_{H} group, as defined herein, appended to the parent molecular moiety through a carbonyl group, as defined herein. Representative examples of (NR_{G}R_{H})carbonyl include, but are not limited to, aminocarbonyl, methylaminocarbonyl, acetylaminocarbonyl, acetylmethylaminocarbonyl, benzylaminocarbonyl, butylaminocarbonyl, 3-methylphenylaminocarbonyl, and phenylaminocarbonyl.

The term “(NR_{G}R_{H})sulfonyl” as used herein, means a —NR_{G}R_{H} group, as defined herein, appended to the parent molecular moiety through a sulfonyl group, as defined herein. Representative examples of (NR_{G}R_{H})sulfonyl include, but are not limited to, aminosulfonyl and dimethylaminosulfonyl.

The term “—NR_{J}R_{K}” as used herein, means two groups, R_{J} and R_{K}, which are appended to the parent molecular moiety through a nitrogen atom. R_{J} and R_{K} are each independently a member selected from the group consisting of hydrogen, alkoxycarbonyl, alkyl, alkylcarbonyl, alkoxysulfonyl, alkylsulfonyl, aryl, arylalkyl, and formyl. Representative examples of —NR_{J}R_{K} include, but are not limited to, amino, ethylamino, benzylamino, dimethylamino, methylamino, tert-butoxycarbonylamino, and propylamino.

The term “(NR_{J}R_{K})alkyl” as used herein, means a —NR_{J}R_{K} group, as defined herein, appended to the parent molecular moiety through an alkyl group, as defined herein. Representative examples of (NR_{J}R_{K})alkyl include, but are not limited to, 2-aminoethyl, 2-(dimethylamino)ethyl, 2-ethylaminoethyl, and 2-(tert-butoxycarbonylamino)ethyl.

The term “(NR_{J}R_{K})carbonyl” as used herein, means a —NR_{J}R_{K} group, as defined herein, appended to the parent molecular moiety through a carbonyl group, as defined herein. Representative examples of (NR_{J}R_{K})carbonyl include, but are not limited to, aminocarbonyl, methylaminocarbonyl, acetylaminocarbonyl, acetylmethylaminocarbonyl, benzylaminocarbonyl, butylaminocarbonyl, 3-methylphenylaminocarbonyl, propylaminocarbonyl, and phenylaminocarbonyl.

The term “(NR_{J}R_{K})sulfonyl” as used herein, means a —NR_{J}R_{K} group, as defined herein, appended to the parent molecular moiety through a sulfonyl group, as defined herein. Representative examples of (NR_{J}R_{K})sulfonyl include, but are not limited to, aminosulfonyl, methylaminosulfonyl, acetylaminosulfonyl, acetylmethylaminosulfonyl, benzylaminosulfonyl, butylaminosulfonyl, 3-methylphenylaminosulfonyl, propylaminosulfonyl, and phenylaminosulfonyl.

The term “—NR₂₅R₂₆” as used herein, means two groups, R₂₅ and R₂₆, which are appended to the parent molecular moiety through a nitrogen atom. R₂₅ and R₂₆ are each independently a member selected from the group consisting of hydrogen, alkyl, and alkylcarbonyl. Representative examples of —NR₂₅R₂₆ include, but are not limited to, acetylamino, amino, ethylamino, dimethylamino, methylamino, and propylamino.

The term “(NR₂₅R₂₆)carbonyl” as used herein, means a —NR₂₅R₂₆ group, as defined herein, appended to the parent molecular moiety through a carbonyl group, as defined herein. Representative examples of (NR₂₅R₂₆)carbonyl include, but are not limited to, aminocarbonyl, methylaminocarbonyl, acetylaminocarbonyl, acetylmethylaminocarbonyl, butylaminocarbonyl, and propylaminocarbonyl.

The term “—NR₂₇R₂₈” as used herein, means two groups, R₂₇ and R₂₈, which are appended to the parent molecular moiety through a nitrogen atom. R₂₇ and R₂₈ are each independently a member selected from the group consisting of hydrogen, alkoxyalkyl, alkoxyalkylcarbonyl, alkoxycarbonyl, alkoxysulfonyl, alkyl, alkylcarbonyl, alkylsulfonyl, alkylthioalkyl, alkylthioalkylcarbonyl, alkylthiocarbonyl, aryl, arylalkoxyalkyl, arylalkyl, arylcarbonyl, cycloalkyl, cycloalkylalkoxyalkyl, cycloalkylalkyl, cycloalkylcarbonyl, formyl, heteroaryl, heteroarylalkoxyalkyl, heteroarylalkyl, heteroarylcarbonyl, heterocycle, heterocyclealkoxyalkyl, heterocyclealkyl, heterocyclecarbonyl, (NZ₂₃Z₂₄)alkyl, and (NZ₂₃Z₂₄)carbonyl. Representative examples of —NR₂₇R₂₈ include, but are not limited to, amino, ethylamino, benzylamino, dimethylamino, methylamino, tert-butoxycarbonylamino, propylamino, (2-(benzyloxy)ethyl)amino, 4-chlorobenzylamino, 4-cyanobenzylamino, 3,4-dichlorobenzylamino and 4-nitrobenzylamino.

The term “(NR₂₇R₂₈)alkyl” as used herein, means a —NR₂₇R₂₈ group, as defined herein, appended to the parent molecular moiety through an alkyl group, as defined herein. Representative examples of —NR₂₇R₂₈ include, but are not limited to, 2-aminoethyl, 2-(dimethylamino)ethyl, 2-ethylaminoethyl, 2-(tert-butoxycarbonylamino)ethyl, 2-((2-(benzyloxy)ethyl)amino)ethyl, 2-(4-chlorobenzylamino)ethyl, 2-(4-cyanobenzylamino)ethyl, 2-(3,4-dichlorobenzylamino)ethyl, and 2-(4-nitrobenzylamino)ethyl.

The term “(NR₂₇R₂₈)carbonyl” as used herein, means a —NR₂₇R₂₈ group, as defined herein, appended to the parent molecular moiety through a carbonyl group, as defined herein. Representative examples of (NR₂₇R₂₈)carbonyl include, but are not limited to, aminocarbonyl, methylaminocarbonyl, acetylaminocarbonyl, acetylmethylaminocarbonyl, benzylaminocarbonyl, butylaminocarbonyl, 3-methylphenylaminocarbonyl, propylaminocarbonyl, phenylaminocarbonyl, (2-(benzyloxy)ethyl)aminocarbonyl, 4-chlorobenzylaminocarbonyl, 4-cyanobenzylaminocarbonyl, 3,4-dichlorobenzylaminocarbonyl, and 4-nitrobenzylaminocarbonyl.

The term “(NR₂₇R₂₈)sulfonyl” as used herein, means a —NR₂₇R₂₈ group, as defined herein, appended to the parent molecular moiety through a sulfonyl group, as defined herein. Representative examples of (NR₂₇R₂₈)sulfonyl include, but are not limited to, aminosulfonyl, methylaminosulfonyl, acetylaminosulfonyl, acetylmethylaminosulfonyl, benzylaminosulfonyl, butylaminosulfonyl, 3-methylphenylaminosulfonyl, propylaminosulfonyl, phenylaminosulfonyl, (2-(benzyloxy)ethyl)aminosulfonyl, 4-chlorobenzylaminosulfonyl, 4-cyanobenzylaminosulfonyl, 3,4-dichlorobenzylaminosulfonyl, and 4-nitrobenzylaminosulfonyl.

The term “—NR₃₀R₃₁” as used herein, means two groups, R₃₀ and R₃₁, which are appended to the parent molecular moiety through a nitrogen atom. R₃₀ and R₃₁ are each independently a member selected from the group consisting of hydrogen, alkoxycarbonyl, alkyl, alkylcarbonyl, alkoxysulfonyl, alkylsulfonyl, aryl, arylalkyl, and formyl. Representative examples of —NR₃₀R₃₁ include, but are not limited to, amino, ethylamino, benzylamino, dimethylamino, methylamino, tert-butoxycarbonylamino, and propylamino.

The term “(NR₃₀R₃₁)alkyl” as used herein, means a —NR₃₀R₃₁, group, as defined herein, appended to the parent molecular moiety through an alkyl group, as defined herein. Representative examples of (NR₃₀R₃₁)alkyl include, but are not limited to, 2-aminoethyl, 2-(dimethylamino)ethyl, 2-ethylaminoethyl, and 2-(tert-butoxycarbonylamino)ethyl.

The term “(NR₃₀R₃₁)carbonyl” as used herein, means a —NR₃₀R₃₁ group, as defined herein, appended to the parent molecular moiety through a carbonyl group, as defined herein. Representative examples of (NR₃₀R₃₁)carbonyl include, but are not limited to, aminocarbonyl, methylaminocarbonyl, acetylaminocarbonyl, acetylmethylaminocarbonyl, benzylaminocarbonyl, butylaminocarbonyl, 3-methylphenylaminocarbonyl, propylaminocarbonyl, and phenylaminocarbonyl.

The term “(NR₃₀R₃₁)sulfonyl” as used herein, means a —NR₃₀R₃₁ group, as defined herein, appended to the parent molecular moiety through a sulfonyl group, as defined herein. Representative examples of (NR₃₀R₃₁)sulfonyl include, but are not limited to, aminosulfonyl, methylaminosulfonyl, acetylaminosulfonyl, acetylmethylaminosulfonyl, benzylaminosulfonyl, butylaminosulfonyl, 3-methylphenylaminosulfonyl, propylaminosulfonyl, and phenylaminosulfonyl.

The term “—NZ₁Z₂” as used herein, means two groups, Z₁ and Z₂, which are appended to the parent molecular moiety through a nitrogen atom. Z₁ and Z₂ are each independently a member selected from the group consisting of hydrogen, alkoxycarbonyl, alkyl, alkylcarbonyl, alkoxysulfonyl, alkylsulfonyl, aryl, arylalkyl, arylcarbonyl, cycloalkyl, cycloalkylalkyl, cycloalkylcarbonyl, formyl, heteroaryl, heteroarylalkyl, heteroarylcarbonyl, heterocycle, heterocyclealkyl, and heterocyclecarbonyl. Representative examples of —NZ₁Z₂ include, but are not limited to, amino, ethylamino, benzylamino, dimethylamino, methylamino, tert-butoxycarbonylamino, and propylamino.

The term “(NZ₁Z₂)alkyl” as used herein, means a —NZ₁Z₂ group, as defined herein, appended to the parent molecular moiety through an alkyl group, as defined herein. Representative examples of —NZ₁Z₂ include, but are not limited to, 2-aminoethyl, 2-(dimethylamino)ethyl, 2-ethylaminoethyl, and 2-(tert-butoxycarbonylamino)ethyl.

The term “(NZ₁Z₂)carbonyl” as used herein, means a —NZ₁Z₂ group, as defined herein, appended to the parent molecular moiety through a carbonyl group, as defined herein. Representative examples of (NZ₁Z₂)carbonyl include, but are not limited to, aminocarbonyl, methylaminocarbonyl, acetylaminocarbonyl, acetylmethylaminocarbonyl, benzylaminocarbonyl, butylaminocarbonyl, 3-methylphenylaminocarbonyl, propylaminocarbonyl, and phenylaminocarbonyl.

The term “—NZ₃Z₄” as used herein, means two groups, Z₃ and Z₄, which are appended to the parent molecular moiety through a nitrogen atom. Z₃ and Z₄ are each independently a member selected from the group consisting of hydrogen, alkoxycarbonyl, alkyl, alkylcarbonyl, alkoxysulfonyl, alkylsulfonyl, aryl, arylalkyl, arylcarbonyl, cycloalkyl, cycloalkylalkyl, cycloalkylcarbonyl, formyl, heteroaryl, heteroarylalkyl, heteroarylcarbonyl, heterocycle, heterocyclealkyl, and heterocyclecarbonyl. Representative examples of —NZ₃Z₄ include, but are not limited to, amino, ethylamino, dimethylamino, methylamino, tert-butoxycarbonylamino, and propylamino.

The term “(NZ₃Z₄)alkyl” as used herein, means a —NZ₃Z₄ group, as defined herein, appended to the parent molecular moiety through an alkyl group, as defined herein. Representative examples of —NZ₃Z₄ include, but are not limited to, 2-aminoethyl, 2-(dimethylamino)ethyl, 2-ethylaminoethyl, and 2-(tert-butoxycarbonylamino)ethyl.

The term “(NZ₃Z₄)carbonyl” as used herein, means a —NZ₃Z₄ group, as defined herein, appended to the parent molecular moiety through a carbonyl group, as defined herein. Representative examples of (NZ₃Z₄)carbonyl include, but are not limited to, aminocarbonyl, methylaminocarbonyl, acetylaminocarbonyl, acetylmethylaminocarbonyl, benzylaminocarbonyl, butylaminocarbonyl, 3-methylphenylaminocarbonyl, propylaminocarbonyl, and phenylaminocarbonyl.

The term “—NZ₅Z₆” as used herein, means two groups, Z₅ and Z₆, which are appended to the parent molecular moiety through a nitrogen atom. Z₅ and Z₆ are each independently a member selected from the group consisting of hydrogen, alkyl, alkylcarbonyl, and formyl. Representative examples of —NZ₅Z₆ include, but are not limited to, amino, methylamino, acetylamino, and acetylmethylamino.

The term “(NZ₅Z₆)alkyl” as used herein, means a —NZ₅Z₆ group, as defined herein, appended to the parent molecular moiety through an alkyl group, as defined herein. Representative examples of (NZ₅Z₆)alkyl include, but are not limited to, aminomethyl, 2-(methylamino)ethyl, 2-(dimethylamino)ethyl, and 3-(ethylmethylamino)propyl.

The term “—NZ₂₃Z₂₄” as used herein, means two groups, Z₂₃ and Z₂₄, which are appended to the parent molecular moiety through a nitrogen atom. Z₂₃ and Z₂₄ are each independently a member selected from the group consisting of hydrogen, alkoxycarbonyl, alkoxysulfonyl, alkyl, alkylcarbonyl, alkylsulfonyl, aryl, arylalkyl, arylcarbonyl, cycloalkyl, cycloalkylalkyl, cycloalkylcarbonyl, formyl, heteroaryl, heteroarylalkyl, heteroarylcarbonyl, heterocycle, heterocyclealkyl, and heterocyclecarbonyl. Representative examples of —NZ₂₃Z₂₄ include, but are not limited to, amino, ethylamino, benzylamino, dimethylamino, methylamino, tert-butoxycarbonylamino, and propylamino.

The term “(NZ₂₃Z₂₄)alkyl” as used herein, means a —NZ₂₃Z₂₄ group, as defined herein, appended to the parent molecular moiety through an alkyl group, as defined herein. Representative examples of —NZ₂₃Z₂₄ include, but are not limited to, 2-aminoethyl, 2-(dimethylamino)ethyl, 2-ethylaminoethyl, and 2-(tert-butoxycarbonylamino)ethyl.

The term “(NZ₂₃Z₂₄)carbonyl” as used herein, means a —NZ₂₃Z₂₄ group, as defined herein, appended to the parent molecular moiety through a carbonyl group, as defined herein. Representative examples of (NZ₂₃Z₂₄)carbonyl include, but are not limited to, aminocarbonyl, methylaminocarbonyl, acetylaminocarbonyl, acetylmethylaminocarbonyl, benzylaminocarbonyl, butylaminocarbonyl, 3-methylphenylaminocarbonyl, propylaminocarbonyl, and phenylaminocarbonyl.

The term “mercapto” as used herein, means a —SH group.

The term “nitro” as used herein, means a —NO₂ group.

The term “sulfinyl” as used herein, means a —SO— group.

The term “sulfonyl” as used herein, means a —SO₂— group.

The present compounds can exist as therapeutically suitable salts. The term “therapeutically suitable salt,” refers to salts or zwitterions of the compounds which are water or oil-soluble or dispersible, suitable for treatment of disorders without undue toxicity, irritation, and allergic response, commensurate with a reasonable benefit/risk ratio, and effective for their intended use. The salts can be prepared during the final isolation and purification of the compounds or separately by reacting an amino group of the compounds with a suitable acid. Representative salts include acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, formate, isethionate, fumarate, lactate, maleate, methanesulfonate, naphthylenesulfonate, nicotinate, oxalate, pamoate, pectinate, persulfate, 3-phenylpropionate, picrate, oxalate, maleate, pivalate, propionate, succinate, tartrate, trichloroacetic, trifluoroacetic, glutamate, para-toluenesulfonate, undecanoate, hydrochloric, hydrobromic, sulfuric, phosphoric, and the like. The amino groups of the compounds can also be quaternized with alkyl chlorides, bromides, and iodides such as methyl, ethyl, propyl, isopropyl, butyl, lauryl, myristyl, stearyl, and the like.

Basic addition salts can be prepared during the final isolation and purification of the present compounds by reaction of a carboxyl group with a suitable base such as the hydroxide, carbonate, or bicarbonate of a metal cation such as lithium, sodium, potassium, calcium, magnesium, or aluminum, or an organic primary, secondary, or tertiary amine. Quaternary amine salts derived from methylamine, dimethylamine, trimethylamine, triethylamine, diethylamine, ethylamine, tributlyamine, pyridine, N,N-dimethylaniline, N-methylpiperidine, N-methylmorpholine, dicyclohexylamine, procaine, dibenzylamine, N,N-dibenzylphenethylamine, 1-ephenamine, and N,N′-dibenzylethylenediamine, ethylenediamine, ethanolamine, diethanolamine, piperidine, piperazine, and the like, are contemplated as being within the scope of the present invention.

The present compounds can also exist as therapeutically suitable prodrugs. The term “therapeutically suitable prodrug,” refers to those prodrugs or zwitterions which are suitable for use in contact with the tissues of patients without undue toxicity, irritation, and allergic response, are commensurate with a reasonable benefit/risk ratio, and are effective for their intended use. The term “prodrug,” refers to compounds which are rapidly transformed in vivo to the parent compounds of the present invention for example, by hydrolysis in blood.

Asymmetric centers can exist in the present compounds. Individual stereoisomers of the compounds are prepared by synthesis from chiral starting materials or by preparation of racemic mixtures and separation by conversion to a mixture of diastereomers followed by separation or recrystallization, chromatographic techniques, or direct separation of the enantiomers on chiral chromatographic columns. Starting materials of particular stereochemistry are either commercially available or are made by the methods described hereinbelow and resolved by techniques well-known in the art.

Geometric isomers can exist in the present compounds. The invention contemplates the various geometric isomers and mixtures thereof resulting from the disposal of substituents around a carbon-carbon double bond, a cycloalkyl group, or a heterocycle group. Substituents around a carbon-carbon double bond are designated as being of Z or E configuration and substituents around a cycloalkyl or heterocycle are designated as being of cis or trans configuration.

Therapeutic compositions of the present compounds comprise an effective amount of the same formulated with one or more therapeutically suitable excipients. The term “therapeutically suitable excipient,” as used herein, represents a non-toxic, solid, semi-solid or liquid filler, diluent, encapsulating material, or formulation auxiliary of any type. Examples of therapeutically suitable excipients include sugars; cellulose and derivatives thereof; oils; glycols; solutions; buffering, coloring, releasing, coating, sweetening, flavoring, and perfuming agents; and the like. These therapeutic compositions can be administered parenterally, intracisternally, orally, rectally, or intraperitoneally.

Liquid dosage forms for oral administration of the present compounds comprise formulations of the same as emulsions, microemulsions, solutions, suspensions, syrups, and elixirs. In addition to the compounds, the liquid dosage forms can contain diluents and/or solubilizing or emulsifying agents. Besides inert diluents, the oral compositions can include wetting, emulsifying, sweetening, flavoring, and perfuming agents.

Injectable preparations of the present compounds comprise sterile, injectable, aqueous and oleaginous solutions, suspensions or emulsions, any of which can be optionally formulated with parenterally suitable diluents, dispersing, wetting, or suspending agents. These injectable preparations can be sterilized by filtration through a bacterial-retaining filter or formulated with sterilizing agents which dissolve or disperse in the injectable media.

Regulation of the effects of ghrelin by the compounds of the present invention can be delayed by using a liquid suspension of crystalline or amorphous material with poor water solubility. The rate of absorption of the compounds depends upon their rate of dissolution which, in turn, depends on their crystalline form. Delayed absorption of a parenterally administered compound can be accomplished by dissolving or suspending the compound in oil. Injectable depot forms of the compounds can also be prepared by microencapsulating the same in biodegradable polymers. Depending upon the ratio of compound to polymer and the nature of the polymer employed, the rate of release can be controlled. Depot injectable formulations are also prepared by entrapping the compounds in liposomes or microemulsions which are compatible with body tissues.

Solid dosage forms for oral administration of the present compounds include capsules, tablets, pills, powders, and granules. In such forms, the compound is mixed with at least one inert, therapeutically suitable excipient such as a carrier, filler, extender, disintegrating agent, solution retarding agent, wetting agent, absorbent, or lubricant. With capsules, tablets, and pills, the excipient can also contain buffering agents. Suppositories for rectal administration can be prepared by mixing the compounds with a suitable non-irritating excipient which is solid at ordinary temperature but fluid in the rectum.

The present compounds can be micro-encapsulated with one or more of the excipients discussed previously. The solid dosage forms of tablets, dragees, capsules, pills, and granules can be prepared with coatings and shells such as enteric and release-controlling. In these forms, the compounds can be mixed with at least one inert diluent and can optionally comprise tableting lubricants and aids. Capsules can also optionally contain opacifying agents which delay release of the compounds in a desired part of the intestinal tract.

Transdermal patches have the added advantage of providing controlled delivery of the present compounds to the body. Such dosage forms are prepared by dissolving or dispensing the compounds in the proper medium. Absorption enhancers can also be used to increase the flux of the compounds across the skin, and the rate of absorption can be controlled by providing a rate controlling membrane or by dispersing the compounds in a polymer matrix or gel.

Disorders that may be regulated by ghrelin are treated or prevented in a patient by administering to the patient, a therapeutically effective amount of a compound of the present invention in such an amount and for such time as is necessary to achieve the desired result. The term “therapeutically effective amount,” refers to a sufficient amount of a compound to effectively emeliorate disorders reglulated by ghrelin at a reasonable benefit/risk ratio applicable to any medical treatment. The specific therapeutically effective dose level for any particular patient will depend upon a variety of factors including the disorder being treated and the severity of the disorder; the activity of the compound employed; the specific composition employed; the age, body weight, general health, sex, and diet of the patient; the time of administration, route of administration, rate of excretion; the duration of the treatment; and drugs used in combination or coincidental therapy.

The total daily dose of the present compounds in single or divided doses can be in amounts, for example, from 0.01 to 50 mg/kg body weight or more usually from 0.1 to 25 mg/kg body weight. In general, treatment regimens comprise administration to a patient in need of such treatment from about 10 mg to about 1000 mg of the compounds per day in single or multiple doses.

### Determination of Biological Activity

Primary Radiolabelled Ligand Competition Binding Assay

Ghrelin binding assays were performed with membrane preparations. CHO—K cells expressing human ghrelin receptor (Euroscreen) were suspended in sucrose buffer (0.25 M sucrose, 10 mM hepes pH 7.4, 1 mM PMSF, 5 ug/ml pepstain-A, 3 mM EDTA and 0.025% bacitracin) and disrupted by sonication using a vibra cell (Sonics and Materials Inc.) on 70% duty cycle in 15-second pulses on ice for 2.5 min. The homogenate was centrifuged at 60,000×g for 60 minutes and pellets were suspended in tris buffer (20 mM tris pH 7.4, 5 ug/ml pepstatin-A, 0.1 mM PMSF and 3 mM EDTA). Binding reactions contained 1 μg membrane as determined by BCA protein assay (Pierce), 0.1 nM [¹²⁵ I]-ghrelin (PerkinElmer) with or without compound addition in 100 μl of binding buffer (25 mM Hepes pH 7.4, 1 mM CaCl₂, 5 mM MgSO₄ and 0.5% protease free BSA). Incubations were carried out at room temperature for 2 hr and were terminated by filtration using Filtermate Harvester (PerkinElmer) onto GF/C filter plates (Millipore) previously soaked in 0.5% polyethylenimine for 2 hours. Bound [¹²⁵I]-ghrelin was determined by scintillation counting using Top Count NXT (PerkinElmer). The effects of compound were expressed as % inhibiton of [¹²⁵I]-ghrelin binding. Sigmoidal curves were fitted by Assay Explorer (MDL) software and IC₅₀ values determined.

The compounds of the present invention were found to inhibit the binding of radio-labeled ghrelin to ghrelin receptor with IC₅₀ in a range of about 0.0001 μM to about 10 μM in the binding assay. In a preferred range, the compounds inhibit the binding of radio-labeled ghrelin to ghrelin receptor with IC₅₀ in a range of about 0.0001 μM to about 1.0 μM; In a more preferred range, the compounds inhibit the binding of radio-labeled ghrelin to ghrelin receptor with IC₅₀ in a range of about 0.0001 μM to about 0.1 μM.

Secondary Fluorescent Calcium Indicator Assay (FLIPR)

CHO—K cells expressing human GHS receptor (Euroscreen) were plated in black 96-well plates with clear bottom (Costar) and cultured to confluency overnight in growth media (Ultra-CHO from BioWhittaker supplemented with 1% dialyzed FCS, 1% penicillin/streptomycin/fungizone, and 400 ug/ml G418 all from Life Technologies) at 37° C. in a humidified cell incubator containing 5% CO₂. Growth media was aspirated and replaced with 100 μl of Dulbecco's phosphate-buffered saline (DPBS) containing 1,000 mg/l D-glucose, 36 mg/l sodium pyruvate, without phenol red (Life Technologies) with 1.14 mM Fluo-4 AM (Molecular Probes) and 0.25 M probenecid (Sigma) for 1 to 3 hours in the dark at room temperature. The dye solution was aspirated and the cells were washed twice with DPBS using the EL-450X cell washer (BioTech). After the last wash, 100 μl of DPBS was added to each well. Cell plates were then transferred to the FLIPR unit (Molecular Probes). Compound additions were 50 μl in duplicate of 4× final concentration in DPBS containing 0.1% BSA and 4% DMSO. Fluorescence emissions from 96 wells were measured simultaneously at excitation and emission wavelength of 488 and 520 nm, respectively for 3 minutes in 1-second intervals for the first minute and 5-second intervals thereafter. During this time agonist responses, if any, were recorded in the absence of ghrelin. Next, 50 μl in duplicate of 4× concentrated ghrelin (Bachem) solution in DPBS containing 0.1% BSA and 4% DMSO were delivered within 1 second by an integrated 96-well pipettor to a final concentration of 1 nM. Fluorescence emissions were measured for another 3 minutes as above. During this time the antagonist effects of compounds on ghrelin-stimulated calcium flux were recorded and expressed as % inhibition of the maximal ghrelin response (10 nM). Sigmoidal curves were fitted by Assay Explorer (MDL) software and IC₅₀ values determined. In addition, the agonist effects of the compounds could also be obtained and expressed as % maximal ghrelin response (10 nM). Sigmoidal curves were fitted by Assay Explorer (MDL) software and EC₅₀ values determined.

For the agonists of ghrelin receptor, the compounds of the present invention were found to stimulate the ghrelin receptor with EC₅₀ in a range of about 0.001 μM to about 10 μM in the FLIPR assays, with the maximal percentage of stimulation exceeds 100%. In a preferred range, the compounds stimulate ghrelin receptor with EC₅₀ in a range of about 0.001 μM to about 1.0 μM, with the maximal percentage of stimulation exceeds 100%; In a more preferred range, the compounds stimulate ghrelin receptor with EC₅₀ in a range of about 0.001 μM to about 0.1 μM, with the maximal percentage of stimulation exceeds 100%.

For the antagonists of ghrelin receptor, the compounds of the present invention were found to inhibit the activition of ghrelin receptor with IC₅₀ in a range of about 0.001 μM to about 10 μM in the FLIPR assays. In a preferred range, the compounds inhibit the activition of ghrelin receptor with IC₅₀ in a range of about 0.001 μM to about 1.0 μM; In a more preferred range, the compounds inhibit the activition of ghrelin receptor with IC₅₀ in a range of about 0.001 μM to about 0.1 μM.

Synthetic Methods

Abbreviations which have been used in the descriptions of the scheme and the examples that follow are: BBr₃ for boron tribromide; m-CPBA for meta-chloroperoxy-benzoic acid; DMF for N,N-dimethylformamide; DMSO for dimethylsulfoxide; DEAD for diethyl azodicarboxylate; EDAC for 1-(3-dimethylaminopropyl)-3-ethylcarbodiimide hydrochloride; HATU for O-(7-azabenzotriazol-1-yl)-N,N,N′N′-tetramethyluronium hexafluorophosphate; HOBT for 1-hydroxybenzotriazole hydrate; NMP for N-methylpyrrolidinone; NCS for N-chlorosuccinimide; MeONa for sodium methoxide; MeOH for methanol; MTBE for methyl tert butyl ether; THF for tetrahydrofuran; TFA for trifluoroacetic acid; TMSCHN₂ for trimethylsilyldiazomethane; TBAF for tetra butylammonium fluoride; Pd(dppf)Cl₂ for (diphenylphospino)ferrocenyl palladium chloride; Ph₃P for triphenylphosphine; Pr₂Net for diisopropyl ethylamine; and TBTU for (benzotriazol-1-yloxy)-dimethylamino-methylene)-dimethyl-ammonium tetrafluoroborate.

The compounds and processes of the present invention will be better understood in connection with the following synthetic schemes which illustrate the methods by which the compounds of the invention may be prepared.

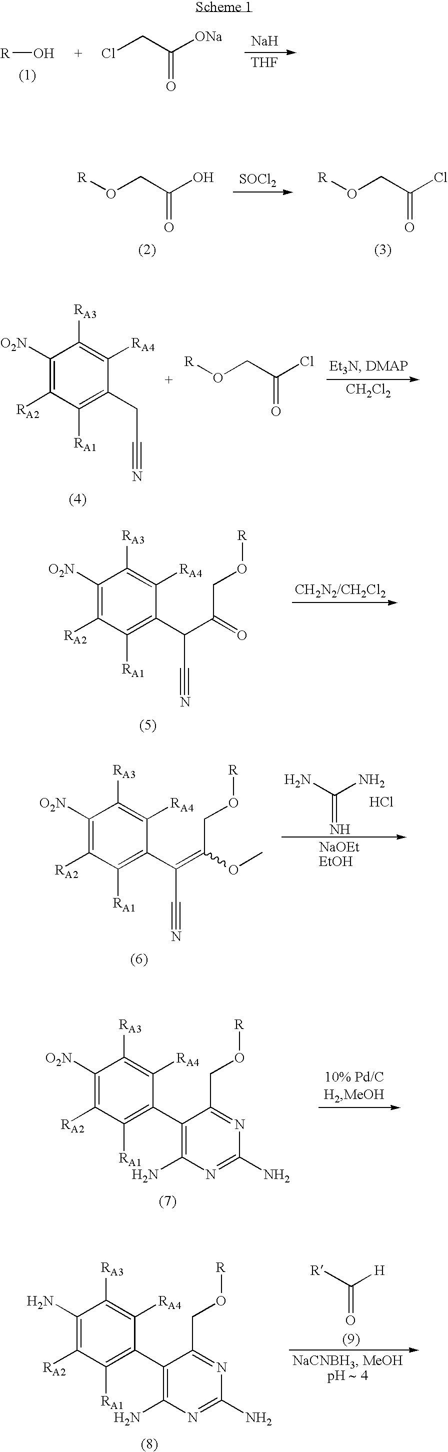

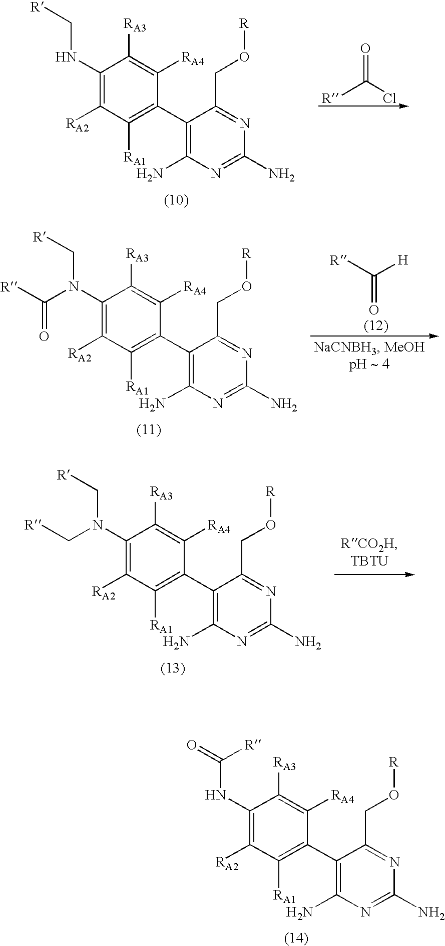

Compounds of the present invention of general formula (10), (11), (12), and (13), wherein R_{A1}, R_{A2}, R_{A3}, and R_{A4}, are as defined in formula (I), R is alkenyl, alkoxyalkoxyalkyl, alkoxyalkyl, alkoxycarbony, alkoxycarbonylalkyl, alkoxysulfonyl, alkylcarbonyl, alkylcarbonylalkyl, alkylthioalkyl, alkynyl, aryl, arylalkoxyalkyl, arylalkyl, arylalkylthioalkyl, aryloxyalkyl, arylthioalkyl, cyanoalkyl, cycloalkenyl, cycloalkenylalkoxyalkyl, cycloalkenylalkyl, cycloalkenylalkylthioalkyl, cycloalkenyloxyalkyl, cycloalkenylthioalkyl, cycloalkyl, cycloalkylalkoxyalkyl, cycloalkylalkyl, cycloalkylalkylthioalkyl, cycloalkyloxyalkyl, cycloalkylthioalkyl, heteroaryl, heteroarylalkoxyalkyl, heteroarylalkyl, heteroarylalkylthioalkyl, heteroaryloxyalkyl, heteroarylthioalkyl, heterocycle, heterocyclealkoxyalkyl, heterocyclealkyl, heterocyclealkylthioalkyl, heterocycleoxyalkyl, heterocyclethioalkyl, (NR_{E}R_{H})alkyl, (NR_{E}R_{F})carbonylalkenyl, (NR_{E}R_{F})carbonylalkyl, (NR_{E}R_{F})sulfonyl, or (NR_{E}R_{F})sulfonylalkyl, R′ and R″ are each independently selected from hydrogen, alkoxyalkyl, alkyl, alkylthioalkyl, aryl, arylalkoxyalkyl, arylalkyl, cycloalkyl, cycloalkylalkoxyalkyl, cycloalkylalkyl, heteroaryl, heteroarylalkoxyalkyl, heteroarylalkyl, heterocycle, heterocyclealkoxyalkyl, or heterocyclealkyl, and R_{E} and R_{F} are as defined in formula (I), can be prepared as described in Scheme 1. Phenols or alcohols of general formula (1) can be treated with sodium chloroacetate to provide acids of general formula (2) Acids of general formula (2) can be treated with thionyl chloride to provide acid chlorides of general formula (3). Acid chlorides of general formula (3) can be treated with cyano compounds of general formula (4) to provide esters of general formula (5). Esters of general formula (5) can be treated with diazomethane followed by treatment with guanidine to provide nitrophenylpyrimidines of general formula (7). Nitrophenylpyrimidines of general formula (7) can be reduced under conditions well known to those of skill in the art including, but not limited to, a palladium catalyst under about 1 atmosphere of hydrogen to provide aminophenylpyrimidines of general formula (8). Aminophenylpyrimidines of general formula (8) can be treated with aldehydes of general formula (9) (or ketones) under reductive amination conditions to provide secondary-aminophenylpyrimidines of general formula (10). Secondary-aminophenylpyrimidines of general formula (10) can be coupled with acids, acid chlorides, or carbonyl compounds to provide compounds of general formula (11), (13), and (14).

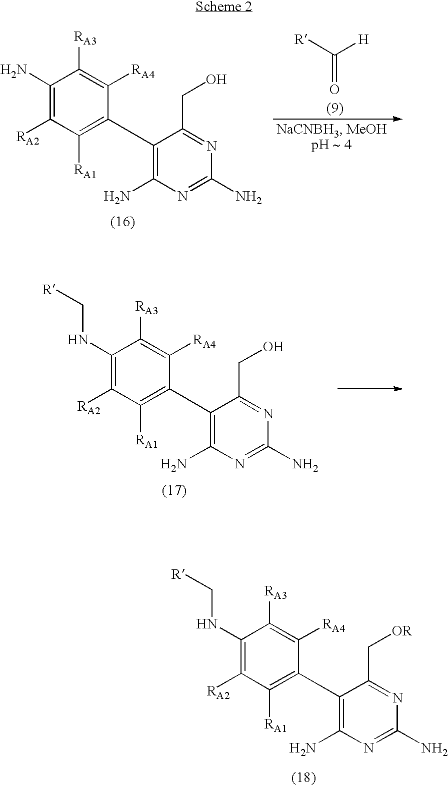

Compounds of the present invention of general formula (17) and (18), wherein R is alkenyl, alkoxyalkoxyalkyl, alkoxyalkyl, alkoxycarbony, alkoxycarbonylalkyl, alkoxysulfonyl, alkylcarbonyl, alkylcarbonylalkyl, alkylthioalkyl, alkynyl, aryl, arylalkoxyalkyl, arylalkyl, arylalkylthioalkyl, aryloxyalkyl, arylthioalkyl, cyanoalkyl, cycloalkenyl, cycloalkenylalkoxyalkyl, cycloalkenylalkyl, cycloalkenylalkylthioalkyl, cycloalkenyloxyalkyl, cycloalkenylthioalkyl, cycloalkyl, cycloalkylalkoxyalkyl, cycloalkylalkyl, cycloalkylalkylthioalkyl, cycloalkyloxyalkyl, cycloalkylthioalkyl, heteroaryl, heteroarylalkoxyalkyl, heteroarylalkyl, heteroarylalkylthioalkyl, heteroaryloxyalkyl, heteroarylthioalkyl, heterocycle, heterocyclealkoxyalkyl, heterocyclealkyl, heterocyclealkylthioalkyl, heterocycleoxyalkyl, heterocyclethioalkyl, (NR_{E}R_{H})alkyl, (NR_{E}R_{F})carbonylalkenyl, (NR_{E}R_{F})carbonylalkyl, (NR_{E}R_{F})sulfonyl, or (NR_{E}R_{F})sulfonylalkyl, R′ is hydrogen, alkoxyalkyl, alkyl, alkylthioalkyl, aryl, arylalkoxyalkyl, arylalkyl, cycloalkyl, cycloalkylalkoxyalkyl, cycloalkylalkyl, heteroaryl, heteroarylalkoxyalkyl, heteroarylalkyl, heterocycle, heterocyclealkoxyalkyl, or heterocyclealkyl, and R_{A1}, R_{A2}, R_{A3}, R_{A4}, R_{E}, and R_{F} are as defined in formula (I), can be prepared as described in Scheme 2. Aminophenylpyrimidines of general formula (16), prepared as described in Examples 2 and 3 contained herein, can be treated with aldehydes of general formula (9) (or ketones) under standard reductive amination conditions to provide secondary-aminophenylpyrimidines of general formula (17). The hydroxy methyl group can then be alkylated/acylated/sulfonylated to provide compounds of general formula (18).

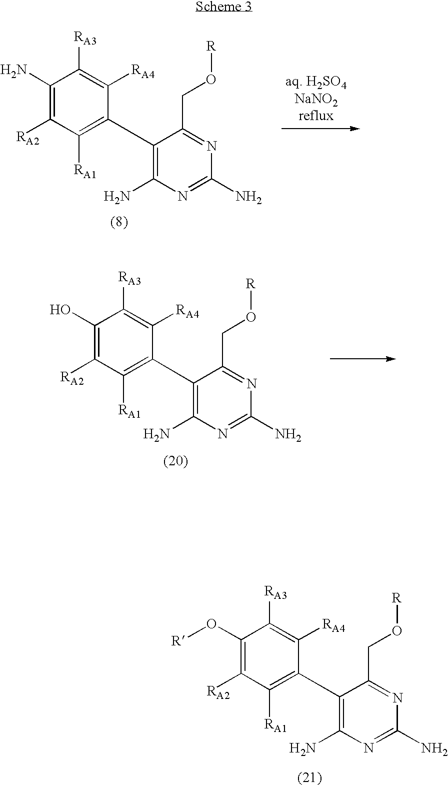

Compounds of the present invention of general formula (20) and (21), wherein R is alkenyl, alkoxyalkoxyalkyl, alkoxyalkyl, alkoxycarbony, alkoxycarbonylalkyl, alkoxysulfonyl, alkylcarbonyl, alkylcarbonylalkyl, alkylthioalkyl, alkynyl, aryl, arylalkoxyalkyl, arylalkyl, arylalkylthioalkyl, aryloxyalkyl, arylthioalkyl, cyanoalkyl, cycloalkenyl, cycloalkenylalkoxyalkyl, cycloalkenylalkyl, cycloalkenylalkylthioalkyl, cycloalkenyloxyalkyl, cycloalkenylthioalkyl, cycloalkyl, cycloalkylalkoxyalkyl, cycloalkylalkyl, cycloalkylalkylthioalkyl, cycloalkyloxyalkyl, cycloalkylthioalkyl, heteroaryl, heteroarylalkoxyalkyl, heteroarylalkyl, heteroarylalkylthioalkyl, heteroaryloxyalkyl, heteroarylthioalkyl, heterocycle, heterocyclealkoxyalkyl, heterocyclealkyl, heterocyclealkylthioalkyl, heterocycleoxyalkyl, heterocyclethioalkyl, (NR_{E}R_{H})alkyl, (NR_{E}R_{F})carbonylalkenyl, (NR_{E}R_{F})carbonylalkyl, (NR_{E}R_{F})sulfonyl, or (NR_{E}R_{F})sulfonylalkyl, R′ is hydrogen, alkoxyalkyl, alkyl, alkylthioalkyl, aryl, arylalkoxyalkyl, arylalkyl, cycloalkyl, cycloalkylalkoxyalkyl, cycloalkylalkyl, heteroaryl, heteroarylalkoxyalkyl, heteroarylalkyl, heterocycle, heterocyclealkoxyalkyl, or heterocyclealkyl, and R_{A1}, R_{A2}, R_{A3}, R_{A4}, R_{E}, and R_{F} are as defined in formula (I), can be prepared as described in Scheme 3. Compounds of general formula (8) can be treated with sodium nitrite and aqueous acid to provide hydroxyphenylpyrimidines of general formula (20). Hydroxyphenylpyrimidines of general formula (20) can be alkylated/acylated/sulfonylated to provide compounds of general formula (21).

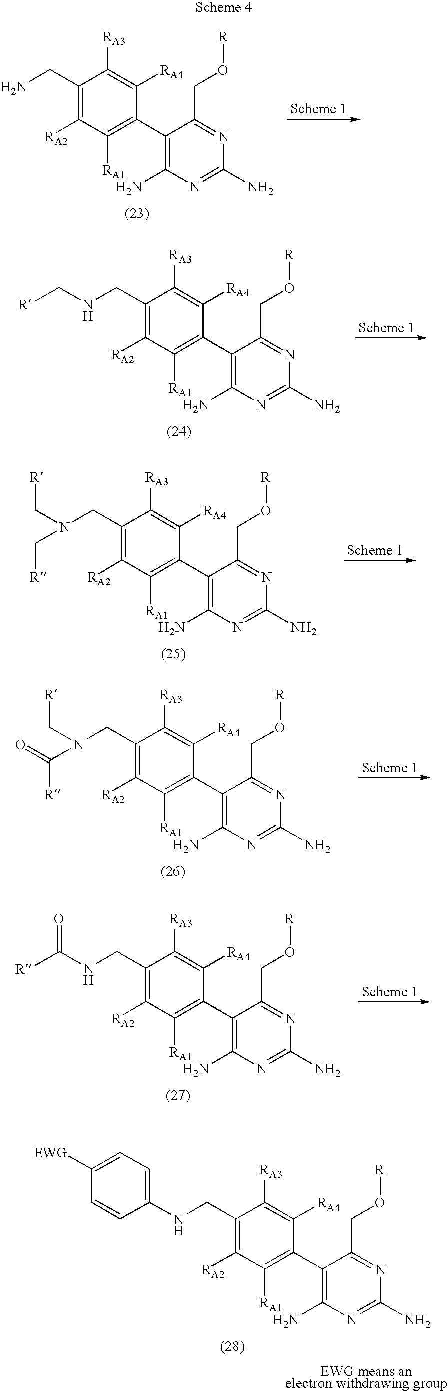

Compounds of the present invention of general formula (23), (24), (25), (26), (27), and (28), wherein R_{A1}, R_{A2}, R_{A3}, and R_{A4}, are as defined in formula (I), R is alkenyl, alkoxyalkoxyalkyl, alkoxyalkyl, alkoxycarbony, alkoxycarbonylalkyl, alkoxysulfonyl, alkylcarbonyl, alkylcarbonylalkyl, alkylthioalkyl, alkynyl, aryl, arylalkoxyalkyl, arylalkyl, arylalkylthioalkyl, aryloxyalkyl, arylthioalkyl, cyanoalkyl, cycloalkenyl, cycloalkenylalkoxyalkyl, cycloalkenylalkyl, cycloalkenylalkylthioalkyl, cycloalkenyloxyalkyl, cycloalkenylthioalkyl, cycloalkyl, cycloalkylalkoxyalkyl, cycloalkylalkyl, cycloalkylalkylthioalkyl, cycloalkyloxyalkyl, cycloalkylthioalkyl, heteroaryl, heteroarylalkoxyalkyl, heteroarylalkyl, heteroarylalkylthioalkyl, heteroaryloxyalkyl, heteroarylthioalkyl, heterocycle, heterocyclealkoxyalkyl, heterocyclealkyl, heterocyclealkylthioalkyl, heterocycleoxyalkyl, heterocyclethioalkyl, (NR_{E}R_{H})alkyl, (NR_{E}R_{F})carbonylalkenyl, (NR_{E}R_{F})carbonylalkyl, (NR_{E}R_{F})sulfonyl, or (NR_{E}R_{F})sulfonylalkyl, R′ and R″ are each independently selected from hydrogen, alkoxyalkyl, alkyl, alkylthioalkyl, aryl, arylalkoxyalkyl, arylalkyl, cycloalkyl, cycloalkylalkoxyalkyl, cycloalkylalkyl, heteroaryl, heteroarylalkoxyalkyl, heteroarylalkyl, heterocycle, heterocyclealkoxyalkyl, or heterocyclealkyl, and R_{E} and R_{F} are as defined in formula (I), can be prepared as described in Scheme 4. Aminomethylphenylpyrimidines of general formula (23), prepared as described in Examples 2 and 64 contained herein, can be alkylated/acylated/sulfonylated/arylated as described in Scheme 1 or as described in the Examples contained herein to provide compounds of general formula (24), (25), (26), and (27), and (28).

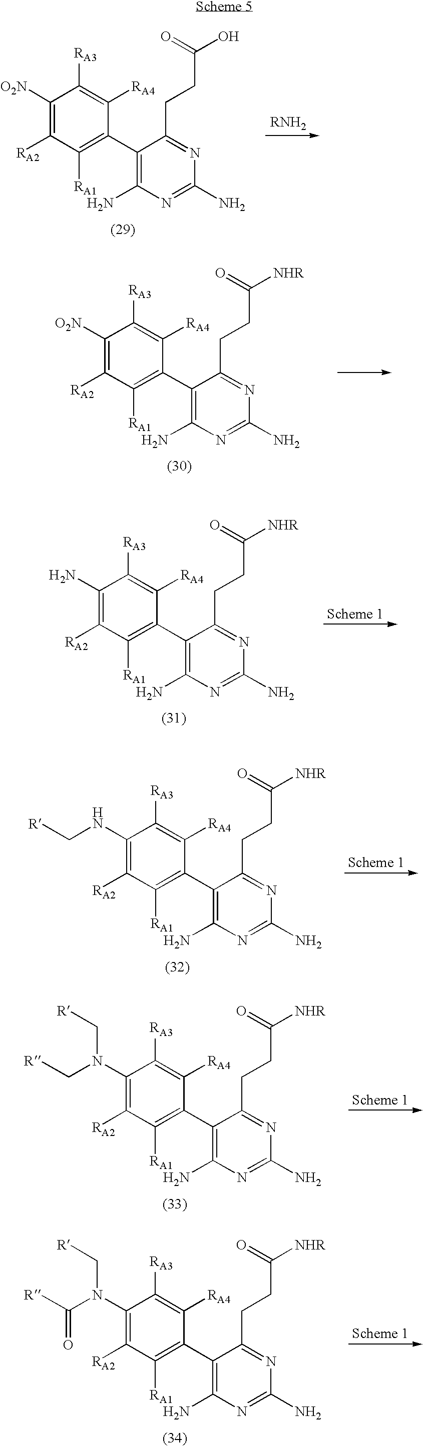

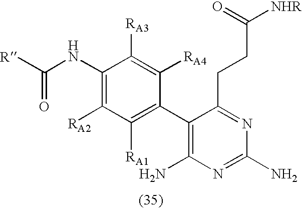

Compounds of the present invention of general formula (31), (32), (33), (34), and (35), wherein R_{A1}, R_{A2}, R_{A3}, and R_{A4}, are as defined in formula (I), R is alkenyl, alkoxyalkoxyalkyl, alkoxyalkyl, alkoxycarbony, alkoxycarbonylalkyl, alkoxysulfonyl, alkylcarbonyl, alkylcarbonylalkyl, alkylthioalkyl, alkynyl, aryl, arylalkoxyalkyl, arylalkyl, arylalkylthioalkyl, aryloxyalkyl, arylthioalkyl, cyanoalkyl, cycloalkenyl, cycloalkenylalkoxyalkyl, cycloalkenylalkyl, cycloalkenylalkylthioalkyl, cycloalkenyloxyalkyl, cycloalkenylthioalkyl, cycloalkyl, cycloalkylalkoxyalkyl, cycloalkylalkyl, cycloalkylalkylthioalkyl, cycloalkyloxyalkyl, cycloalkylthioalkyl, heteroaryl, heteroarylalkoxyalkyl, heteroarylalkyl, heteroarylalkylthioalkyl, heteroaryloxyalkyl, heteroarylthioalkyl, heterocycle, heterocyclealkoxyalkyl, heterocyclealkyl, heterocyclealkylthioalkyl, heterocycleoxyalkyl, heterocyclethioalkyl, (NR_{E}R_{H})alkyl, (NR_{E}R_{F})carbonylalkenyl, (NR_{E}R_{F})carbonylalkyl, (NR_{E}R_{F})sulfonyl, or (NR_{E}R_{F})sulfonylalkyl, R′ and R″ are each independently selected from hydrogen, alkoxyalkyl, alkyl, alkylthioalkyl, aryl, arylalkoxyalkyl, arylalkyl, cycloalkyl, cycloalkylalkoxyalkyl, cycloalkylalkyl, heteroaryl, heteroarylalkoxyalkyl, heteroarylalkyl, heterocycle, heterocyclealkoxyalkyl, or heterocyclealkyl, and R_{E} and R_{F} are as defined in formula (I), can be prepared as described in Scheme 5. Compounds of general formula (29), prepared as described in Example 61 herein, can be treated with amines to provide amides of general formula (30). Amides of general formula (30) can be reduced to aminophenylpyrimidines of general formula (31). Aminophenylpyrimidines of general formula (31) can be alkylated/acylated/sulfonylated as described in Scheme 1 or as described in the Examples contained herein to provide compounds of general formula (32),5 (33), (34), and (35).

The present invention will now be described in connection with certain embodiments which are not intended to limit its scope. On the contrary, the present invention covers all alternatives, modifications, and equivalents as can be included within the scope of the claims. Thus, the following examples, which include preferred embodiments, will illustrate the preferred practice of the present invention, it being understood that the examples are for the purposes of illustration of certain preferred embodiments.

Compounds of the invention were named by ACD/ChemSketch version 5.01 (developed by Advanced Chemistry Development, Inc., Toronto, ON, Canada) or were given names which appeared to be consistent with ACD nomenclature.

### EXAMPLE 1

### 5-{4-[(4-Chlorobenzyl)amino]phenyl}-6-ethylpyrimidine-2,4-diamine

### EXAMPLE 1A

### 2-(4-Nitro-phenyl)-3-oxo-pentanenitrile

To a solution of 8.10 g (50.0 mmol) of 4-nitrophenylacetonitrile in 100 mL of CH₂Cl₂ was added 610 mg (5 mmol) of 4-N,N-dimethylaminopyridine. The solution was cooled with an ice bath, then 8.7 mL (100 mmol) of propionyl chloride was added dropwise to avoid reflux of the solvent. After 45 minutes, the solvent was removed in vacuo, and the residue was taken up in 200 mL of 0.5 M HCl. The mixture was extracted with diethyl ether (3×50 mL), then the combined ether layers were back extracted with water (1×50 mL), brine (1×50 mL), dried over MgSO₄, filtered, and concentrated under reduced pressure to an oil.

The oil was taken up in 250 mL of methanol, and to the solution was added 200 mL of 2M NaOH. The solution was stirred for 15 minutes, then 1 L of water was added, followed by 12M HCl until precipitation was complete. The suspension was extracted with diethyl ether (2×200 mL), then the combined ether layers were back extracted with brine (1×100 mL), dried over MgSO₄, filtered, and concentrated under reduced pressure to provide the titled compound (9.3 g, 85%) as a solid. This material may be used in the next step without further purification, or maybe recrystallized from toluene to give a crystalline product.

### EXAMPLE 1B

### 6-Ethyl-5-(4-nitro-phenyl)-pyrimidine-2,4-diamine

To 1.91g (8.75 mmol) of 2-(4-nitro-phenyl)-3-oxo-pentanenitrile from Example 1A in 20 mL of ethyl acetate was added ethereal CH₂N₂ until excess CH₂N₂ was present. The reaction was concentrated to an oil. This was taken up in 5 mL of ethanol, then treated with a premixed solution of 955 mg (10 mmol) of guanidine hydrochloride and potassium ethoxide (10 mmol) in 14 mL of ethanol. (The guanidine solution contained precipitated KCl). The reaction was stirred at reflux for 2 hours, then concentrated under reduced pressure. The residue was taken up in 20 mL of water and filtered to give a black precipitate. The precipitate was washed with 100 mL of water, recrystallized from 25 mL of ethanol. The recrystallized product was filtered, and washed with 10 mL of cold ethanol to provide the titled compound (700 mg, 27%) as green crystals.

### EXAMPLE 1C

### 5-(4-Amino-phenyl)-6-ethyl-pyrimidine-2,4-diamine

To a solution of 1.95 g (7.52 mmol) of 6-ethyl-5-(4-nitro-phenyl)-pyrimidine-2,4-diamine from Example 1B in 60 mL of glacial acetic acid was added 200 mg of 10% Pd-C. The reaction was stirred under 1 atmosphere of H₂ for 5 hours. The catalyst was filtered, and the solvent was removed under reduced pressure at 40° C. to provide a crystalline solid. The solid was dissolved in 25 mL of water, and the solution was made basic (pH=14) by the addition of 2M NaOH. The precipitate was filtered, and washed with water until the washings were pH=8. The product was dried on the filter to provide 1.55 g (90%) of light yellow crystals.

### EXAMPLE 1D

### 5-{4-[(4-Chlorobenzyl)amino]phenyl}-6-ethylpyrimidine-2,4-diamine

To a solution of 102 mg (0.445 mmol) of 5-(4-amino-phenyl)-6-ethyl-pyrimidine-2,4-diamine from Example 1C in 3 mL of methanol was added a solution of 63 mg (0.45 mmol) of 4-chlorobenzaldehyde in 2 mL of methanol. The solution was stirred at ambient temperature for 10 minutes, then 1 mL of acetic acid was added, followed by 100 mg (1.59 mmol) of sodium cyanoborohydride. The solution was concentrated under reduced pressure to a volume of about 1 mL. The remainder was dissolved in 5 mL of water to which 10 mL of 2M NaOH was added. The formed precipitate was filtered, and washed with water until the washings were pH=8. The precipitate was recrystallized from 1 mL of ethanol to provide 46 mg (29%) of yellow crystals. ¹H NMR (300 MHz, d₆-DMSO) δ 7.40 (m, 4H), 6.84 (d, 2H, J=8.5 Hz), 6.62 (d, 2H, J=8.5 Hz), 6.34 (t, 1H, J=6.1 Hz), 5.72 (s, 2H), 5.26 (bs, 2H), 4.26 (d, 2H, J=5.8 Hz), 2.10 (q, 2H, J=7.6 Hz), 0.94 (t, 3H, J=7.6 Hz); MS (ESI) m/z 354 [M+H]⁺.

### EXAMPLE 2

### 6-[(Benzyloxy)methyl]-5-{4-[(4-chlorobenzyl)amino]phenyl}pyrimidine-2,4-diamine

### EXAMPLE 2A

### 4-Benzyloxy-2-(4-nitro-phenyl)-3-oxo-butyronitrile

4-Nitrophenylacetonitrile (10.0 g, 61.7 mmol), triethylamine (14.5 g, 144 mmol) and 4-(dimethylamino)pyridine (800 mg, 6.56 mmol) were dissolved in CH₂Cl₂ (150 mL). The solution was cooled to 0° C. and benzyloxyacetyl chloride (12.0 g, 64.8 mmol) was added dropwise over a 30 minutes. The reaction mixture was warmed to room temperature and stirred for 2 hours. CH₂Cl₂ was removed under reduced pressure and the mixture was dissolved in ethyl acetate (150 mL) and washed with aqueous NaHCO₃ (150 mL) and aqueous HCl (10%, 2×150 mL). The solcents were removed under reduced pressure to provide crude 4-benzyloxy-2-(4-nitro-phenyl)-3-oxo-butyronitrile (19.6 g). R_{f}=0.11 (50% ethyl acetate in hexanes)

### EXAMPLE 2B

### 6-Benzyloxymethyl-5-(4-nitro-phenyl)-pyrimidine-2,4-diamine

4-Benzyloxy-2-(4-nitro-phenyl)-3-oxo-butyronitrile (9.72 g, 31.4 mmol) was dissolved in CH₂Cl₂ (80 mL) and TMSCHN₂ (30 mL, 2M in Et₂O, 60 mmol) was added slowly. HOAc (glacial) was added dropwise until excess TMSCHN₂ was destroyed as evidenced by the cessation of N₂ evolution. The solution was concentrated under reduced pressure and the residue dissolved in 60 mL EtOH. Guanidine hydrochloride (3.605 g, 37.5 mmol) was mixed with 60 mL EtOH followed by addition of NaOEt in EtOH (14.2 mL, 37.6 mmol). After stirring the guanidine solution for 5 minutes the solution was added to the enol ether/ethanol solution resulting in a very dark, purple mixture. The reaction mixture was heated to reflux for 3 hours. The solution was concentrated under reduced pressure followed by addition of EtOAc (150 mL) and aqueous NaOH (200 mL, 0.5M). The mixture was stirred and the formed precipitate was filtered providing 6-benzyloxymethyl-5-(4-nitro-phenyl)-pyrimidine-2,4-diamine (8.78 g, 79.5%) as a light brown solid.

### EXAMPLE 2C

### 5-(4-Amino-phenyl)-6-benzyloxymethyl-pyrimidine-2,4-diamine

6-Benzyloxymethyl-5-(4-nitro-phenyl)-pyrimidine-2,4-diamine (5.00 g, 14.25 mmol) and Pd(OH)₂/C (600 mg) in MeOH (140 mL) in a heavy walled reaction vessel was charged with H₂ (60 psi) and the mixture shaken at room temperature for 14 hour. The mixture was filtered to remove the catalyst and the solution concentrated to provide 5-(4-amino-phenyl)-6-benzyloxymethyl-pyrimidine-2,4-diamine (4.34 g, 95%) as a light brown solid.

### EXAMPLE 2D

### 6-Benzyloxymethyl-5-[4-(4-chloro-benzylamino)-phenyl]-pyrimidine-2,4-diamine

5-(4-Amino-phenyl)-6-benzyloxymethyl-pyrimidine-2,4-diamine (1.62 g, 5.05 mmol) was dissolved in MeOH/NaOAc/HOAc (80 mL, 1M, pH 4). 4-Chlorobenzaldehyde (851 mg, 6.06 mmol) was added and the mixture stirred for 15 minutes. NaBH₃CN (375 mg, 6.06) was then added and the reaction mixture was stirred for 16 hours at 25° C. EtOAc (180 mL) was added and the mixture was washed with HCl (10%, 75 mL), NaOH (2M, 2×100 mL), and brine (100 mL). The crude material was purified by silica gel chromatography (EtOAc to 10% MeOH in EtOAc gradient) providing 6-benzyloxymethyl-5-[4-(4-chloro-benzylamino)-phenyl]-pyrimidine-2,4-diamine (1.56 g, 69.5%). ¹H NMR (300 MHz, DMSO-d₆) δ 3.94 (s, 2 H), 4.28 (d, J=5.8 Hz, 2 H), 4.32 (s, 2 H), 5.49 (s, 2 H), 5.87 (s, 2 H), 6.37 (t, J=5.9 Hz, 1 H), 6.60 (d, J=8.5 Hz, 2 H), 6.89 (d, J=8.5 Hz, 2 H), 7.17 (m, 2 H), 7.27 (m, 3 H), and 7.40 (m, 4 H). MS (ESI) positive ion 446 (M+H)⁺; negative ion 444 (M−H)⁻.

### EXAMPLE 3

### 5-{4-[(4-Chlorobenzyl)amino]phenyl}-6-(methoxymethyl)pyrimidine-2,4-diamine

### EXAMPLE 3A

### [2,6-Diamino-5-(4-amino-phenyl)-pyrimidin-4-yl]-methanol

6-Benzyloxymethyl-5-(4-nitro-phenyl)-pyrimidine-2,4-diamine (550 mg, 1.57 mmol) from example 2B and Pd(OH)₂/C (550 mg) were mixed in MeOH (10 mL) under an atmosphere of nitrogen. 12 M HCl (0.75 mL, 9.0 mmol) was added to the mixture in a heavy walled vessel which was then charged with H₂ (60 psi). The mixture was shaken for 2 hours at room temperature. The catalyst was filtered, the reaction mixture washed with NaOH (2M, 50 mL), extracted in EtOAC (150 mL), and the solvent removed under reduced pressure to provide [2,6-diamino-5-(4-amino-phenyl)-pyrimidin-4-yl]-methanol (360 mg, 99%).

### EXAMPLE 3B

### {2,6-Diamino-5-[4-(4-chloro-benzylamino)-phenyl]-pyrimidin-4-yl}-methanol

[2,6-Diamino-5-(4-amino-phenyl)-pyrimidin-4-yl]-methanol from Example 3A (360 mg, 1.56 mmol) and 4-chlorobenzaldehyde (265 mg, 1.88 mmol) were dissolved in MeOH/HOAc/NaOAc (1M, 2 mL) buffer and stirred for 5 minutes. The pH was adjusted to 4, NaBH₃CN (117 mg, 1.88 mmol) was added and the mixture was for stirred 16 hours at room temperature. The mixture was concentrated under reduced pressure and purified on silica gel (5% MeOH in EtOAc) providing {2,6-diamino-5-[4-(4-chloro-benzylamino)-phenyl]-pyrimidin-4-yl}-methanol (393 mg, 71.0%) as a white solid.

### EXAMPLE 3C

### 5-{4-[(4-Chlorobenzyl)amino]phenyl}-6-(methoxymethyl)pyrimidine-2,4-diamine

{2,6-Diamino-5-[4-(4-chloro-benzylamino)-phenyl]-pyrimidin-4-yl}-methanol from Example 3B (30 mg, 0.085 mmol) was dissolved in MeOH (0.3 mL). NaH (5.0 mg, 60% dispersion in mineral oil, 0.13 mmol) was added and stirred until H₂ evolution ceased. Mel (12 mg, 0.085 mmol) was added and the mixture was stirred 16 hours. Purification was performed by reverse phase HPLC (5-100% CH₃CN in aq. NH₄Oac) providing 5-[4-(4-chloro-benzylamino)-phenyl]-6-methoxymethyl-pyrimidine-2,4-diamine (12 mg, 38%) as an off-white solid. ¹H NMR (300 MHz, DMSO-d₆) δ 2.21 (d, J=2.03 Hz, 3 H), 3.10 (s, 3 H), 3.82 (s, 2 H), 4.27 (d, J=5.76 Hz, 2 H), 5.47 (s, 2 H), 5.86 (s, 2 H), 6.36 (t, J=5.93 Hz, 1 H), 6.61 (d, J=8.48 Hz, 2 H), 6.87 (d, J=8.48 Hz, 2 H), and 7.40 (m, 4 H). MS (ESI) positive ion 370 (M+H)⁺; negative ion 368 (M−H)⁻.

### EXAMPLE 4

### 5-{4-[(4-Chlorobenzyl)amino]phenyl}-6-{[(2-fluoro-3-methylbenzyl)oxy]methyl}pyrimidine-2,4-diamine

[2,6-diamino-5-(4-amino-phenyl)-pyrimidin-4-yl]-methanol from Example 3B (36 mg, 0.10 mmol) and sodium tert-butoxide (15 mg, 0.156 mmol) in DMF (0.3 mL) were stirred for 20 minutes at room temperature followed by the addition of 2-fluoro-3-methyl benzyl bromide (18 mg, 0.09 mmol). The reaction mixture was stirred for 1.5 hours followed by the addition of 1 M HCl (0.1 mL) and MeOH (1.5 mL). The mixture was filtered and the resulting precipitate purified by reverse phase HPLC (0-70% CH₃CN in aq. NH₄OAc) to provide the title compound (12 mg, 28%) as an off white solid. ¹H NMR (300 MHz, DMSO-d₆) δ 3.96 (s, 2 H), 4.27 (d, J=6.10 Hz, 2 H), 4.37 (s, 2 H), 5.55 (s, 2 H), 5.93 (s, 2 H), 6.37 (t, J=5.93 Hz, 1 H), 6.58 (d, J=8.82 Hz, 2 H), 6.88 (d, J=8.48 Hz, 2 H), 6.97 (t, J=7.46 Hz, 1 H), 7.06 (m, 1 H), 7.17 (m, 1 H), and 7.39 (m, 4 H). MS (ESI) positive ion 478 (M+1)⁺; negative ion 476 (M−1)⁻.

### EXAMPLE 5

### 5-{4-[(4-Chlorobenzyl)amino]phenyl}-6-(3-phenylpropyl)pyrimidine-2,4-diamine

### Example 5A

### 5-(4-Amino-phenyl)-6-(3-phenyl-propyl)-pyrimidine-2,4-diamine

The titled compound was prepared according to the procedure described in Example 2, substituting 4-phenyl-butyryl chloride for benzyloxyacetyl chloride used in Example 2.

### EXAMPLE 5B

### 5-{4-[(4-Chlorobenzyl)amino]phenyl}-6-(3-phenylpropyl)pyrimidine-2,4-diamine

To a stirred solution of 6-(4-amino-phenyl)-5-(3-phenyl-propyl)-pyrimidine-2,4-diamine from Example 5A (210 mg, 0.658 mmol) in MeOH (3.3 mL) was added 4-chlorobenzaldehyde (92 mg, 0.658 mmol). After 30 minutes at room temperature, the reaction was cooled to 0° C. Glacial acetic acid (0.185 mL, 3.3 mmol) was added followed by NaCNBH₃ (45 mg, 0.724 mmol). The mixture was warmed to room temperature over 1.5 hour. The solvent was removed under reduced pressure, the residue was taken up in aqueous NaHCO₃ (10 mL) and washed with EtOAc (3×10 mL). The combined organic layers were washed with brine (10 mL), dried over MgSO₄, filtered and concentrated under reduced pressure. Recrystallization from EtOH resulted in a pale yellow solid (145 mg, 50%). ¹H NMR (300 MHz, DMSO-d₆) δ 7.31-7.44 (m, 5 H), 7.09-7.21 (m, 3H), 7.01 (d, J=6.78 Hz, 2H), 6.82 (d, J=8.48 Hz, 2H), 6.61 (d, J=8.48, 2 H), 6.34 (t, J=5.93, 1H), 5.72 (s, 2H), 5.26 (s, 2H), 4.27 (d, J=6.10 Hz, 2 H), 2.40 (t, 7.63, 2H), 2.11 (t, J=7.5 Hz, 2 H), 1.72-1.77 (m, 2H). MS (ESI) positive ion 446 (M+H)⁺; negative ion 444 (M−H)⁻.

### EXAMPLE 6

### 4-({[4-(2,4-Diamino-6-ethylpyrimidin-5-yl)phenyl]amino}methyl)benzonitrile

Synthesis was performed using a PE Biosystems (Applied Biosystems) Solaris 530 organic synthesizer. 4-Cyanobenzyl alcohol (0.6 mmol) was dissolved in 3 mL DMA then transferred to a 4 mL vial containing 0.5 mmol of Dess-Martin Reagent. The vial was shaken to ensure mixing then used directly. A round bottom flask was charged with 3 equivalents of MP-BH₃CN. The block was placed on the Solaris 530 and 1 equivalent of 5-(4-amino-phenyl)-6-ethyl-pyrimidine-2,4-diamine from Example 1C (dissolved in 1:1 MeOH/CH₂Cl₂) was added to the round bottom flask. The oxidized 4-cyanobenzyl alcohol was then added (2 eq) followed by 3 equivalents of a solution of HOAc in 1:1 MeOH/CH₂Cl₂. The block was then heated to 55C overnight. The mixture was transferred with MeOH to a 20 mL vial containing scavenger resins PS-TsNHNH₂ and MP-Carbonate (3 eq each). The resins were filtered and the product concentrated. Purification by Reverse Phase HPLC to provided the titled compound. ¹H NMR (500 MHz, DMSO-d₆) δ ppm 0.94 (t, J=7.49 Hz, 3 H), 2.11 (q, J=7.59 Hz, 2 H), 4.38 (d, J=5.93 Hz, 2 H), 5.32 (s, 2 H), 5.77 (s, 2 H), 6.44 (t, J=5.93 Hz, 1 H), 6.61 (d, J=8.42 Hz, 2 H), 6.85 (d, J=8.42 Hz, 2 H), 7.58 (d, J=8.11 Hz, 2 H), 7.80 (d, J=8.11 Hz, 2 H); MS (ESI) positive ion 345(M+H)⁺.

### EXAMPLE 7

### 5-{4-[(3,4-Dichlorobenzyl)amino]phenyl}-6-ethylpyrimidine-2,4-diamine

The titled compound was prepared according to the procedure described in Example 6, substituting 3,4-dichlorobenzyl alcohol for 4-cyanobenzyl alcohol used in Example 6. ¹H NMR (500 MHz, DMSO-d₆) δ ppm 0.97 (t, J=7.64 Hz, 3 H), 2.16 (q, J=7.69 Hz, 2 H), 4.30 (d, J=5.93 Hz, 2 H), 6.46 (m, 1 H), 6.64 (d, J=8.42 Hz, 2 H), 6.89 (d, J=8.42 Hz, 2 H), 7.39 (dd, J=8.26, 1.72 Hz, 1 H), 7.62 (m, 2 H); MS (ESI) positive ion 390(M+H)⁺.

### EXAMPLE 8

### 5-{4-[(4-Chlorobenzyl)amino]phenyl}-6-(phenoxymethyl)pyrimidine-2,4-diamine

The title compound was synthesized according to the procedure described in Example 2, substituting phenoxyacetyl chloride for benzyloxyacetyl chloride. ¹H NMR (300 MHz, DMSO-d₆) δ 4.24 (d, J=6.10 Hz, 2 H), 4.45 (s, 2 H), 5.58 (s, 2 H), 5.93 (s, 2 H), 6.35 (t, J=5.93 Hz, 1 H), 6.58 (d, J=8.48 Hz, 2 H), 6.76 (m, 2 H), 6.87 (m, 1 H), 6.92 (d, J=8.48 Hz, 2 H), 7.19 (m, 2 H), and 7.37 (m, 4 H). MS (ESI) positive ion 432 (M+H)⁺; negative ion 430 (M−H)⁻.

### EXAMPLE 9

### 5-{4-[(4-Chlorobenzyl)amino]phenyl}-6-propylpyrimidine-2,4-diamine

### EXAMPLE 9A

### 5-(4-Amino-phenyl)-6-propyl-pyrimidine-2,4-diamine

The titled compound was prepared according to the procedure described in Example 2, substituting butyryl chloride for benzyloxyacetyl chloride used in Example 2.

### EXAMPLE 9B

### 5-{4-[(4-chlorobenzyl)amino]phenyl}-6-propylpyrimidine-2,4-diamine

To a stirred solution of 6-(4-amino-phenyl)-5-propyl-pyrimidine-2,4-diamine from Example 9A (100 mg, 0.411 mmol) in MeOH (2.0 mL) was added 4-chlorobenzaldehyde (57 mg, 0.411 mmol). The mixture was stirred for 20 minutes at room temperature then cooled to 0° C. Glacial acetic acid (0.100 mL, 1.7 mmol) was added followed by NaCNBH₃ (28 mg, 0.452 mmol). The mixture was warmed to room temperature over 1 hour and the solvent removed under reduced pressure. The residue was taken up in aqueous NaHCO₃ (5 mL) and washed with EtOAc (2×5 mL). The combined organic layers were washed with brine (5 mL), dried over MgSO₄, filtered and concentrated under reduced pressure. The residue was triturated from 1 mL of 10:1:0.1 CH₂Cl₂:MeOH:NH₄OH and filtered to provide a white solid (77 mg, 52%). ¹H NMR (300 MHz, DMSO-d₆) δ 7.40-7.44 (m, 4 H), 6.83 (d, J=8.48 Hz, 2H), 6.62 (d, J=8.48 Hz, 2H), 6.34 (t, J=6.10 Hz, 1H), 5.83 (s, 2H), 5.40 (s, 2H), 4.26 (d, J=5.76 Hz, 2H), 2.06-2.11 (m, 2H), 1.40-1.47 (m, 2H), 0.716 (t, J=7.29 Hz, 3H). MS (ESI) positive ion 368 (M+H)⁺; negative ion 366 (M−H)⁻.

### EXAMPLE 10

### 5-{4-[(4-Chlorobenzyl)amino]phenyl}-6-{[(3-methylbenzyl)oxy]methyl}pyrimidine-2,4-diamine

To 1-(bromomethyl)-3-methylbenzene (0.06 mmol) in 0.31 mL of DMF was added a solution of {2,6-diamino-5-[4-(4-chloro-benzylamino)-phenyl]-pyrimidin-4-yl}-methanol from Example 3B (0.07 mmol) and NaOtBu (0.105 mmol) in 0.6 mL DMF. The mixture was heated to 55° C. overnight then concentrated under reduced pressure to dryness. Purification by Reverse Phase Chromatography provided the titled compound. ¹H NMR (500 MHz, DMSO-d₆) δ ppm 2.27 (m, 3 H), 3.31 (m, 4 H), 3.96 (m, 2 H), 4.27 (m, 4 H), 6.35 (t, J=6.08 Hz, 1 H), 6.60 (d, J=8.42 Hz, 2 H), 6.89 (d, J=8.73 Hz, 2 H), 6.96 (d, J=7.49 Hz, 1 H), 6.99 (s, 1 H), 7.04 (d, J=7.49 Hz, 1 H), 7.14 (t, J=7.64 Hz, 1 H), 7.39 (m, 4 H); MS (ESI) positive ion 460(M+H)⁺.

### EXAMPLE 11

### 5-{4-[(4-Chlorobenzyl)amino]phenyl}-6-{[(2-methoxybenzyl)oxy]methyl}pyrimidine-2,4-diamine

The titled compound was prepared according to the procedure described in Example 10, substituting 2-methoxybenzyl bromide for 1-(bromomethyl)-3-methylbenzene used in Example 10. ¹H NMR (500 MHz, DMSO-d₆) δ ppm 1.93 (m, 3 H), 3.73 (s, 2 H), 3.96 (s, 2 H), 4.27 (d, J=5.93 Hz, 2 H), 4.33 (s, 2 H), 5.85 (s, 2 H), 6.34 (t, J=5.93 Hz, 1 H), 6.59 (d, J=8.42 Hz, 2 H), 6.83 (t, J=7.49 Hz, 1 H), 6.91 (m, 3 H), 7.13 (d, J=7.48 Hz, 1 H), 7.22 (m, 1 H), 7.40 (q, J=8.52 Hz, 4 H); MS (ESI) positive ion 476(M+H)⁺.

### EXAMPLE 12

### 5-{4-[(4-Chlorobenzyl)amino]phenyl}-6-{[(3-methoxybenzyl)oxy]methyl}pyrimidine-2,4-diamine

The titled compound was prepared according to the procedure described in Example 10, substituting 3-methoxybenzyl bromide for 1-(bromomethyl)-3-methylbenzene used in Example 10. ¹H NMR (500 MHz, DMSO-d₆) δ ppm 3.72 (d, J=6.24 Hz, 3 H), 3.95 (s, 2 H), 4.27 (d, J=5.93 Hz, 2 H), 4.30 (s, 2 H), 5.52 (m, 1 H), 5.86 (s, 2 H), 6.34 (t, J=6.08 Hz, 1 H), 6.60 (d, J=8.42 Hz, 2 H), 6.78 (m, 3 H), 6.89 (d, J=8.73 Hz, 2 H), 7.17 (t, J=7.80 Hz, 1 H), 7.39 (m, 4 H); MS (ESI) positive ion 476(M+H)⁺.

### EXAMPLE 13

### 5-{4-[(4-Chlorobenzyl)amino]phenyl}-6-{[(4-methoxybenzyl)oxy]methyl}pyrimidine-2,4-diamine

The titled compound was prepared according to the procedure described in Example 10, substituting 4-methoxybenzyl bromide for 1-(bromomethyl)-3-methylbenzene used in Example 10. ¹H NMR (500 MHz, DMSO-d₆) δ ppm 3.73 (s, 3 H), 3.90 (s, 2 H), 4.23 (s, 2 H), 4.28 (d, J=5.93 Hz, 2 H), 5.47 (m, 2 H), 5.85 (s, 2 H), 6.34 (t, J=6.08 Hz, 1 H), 6.60 (d, J=8.42 Hz, 2 H), 6.86 (dd, J=28.23, 8.58 Hz, 4 H), 7.10 (d, J=8.73 Hz, 2 H), 7.40 (m, 4 H); MS (ESI) positive ion 476(M+H)⁺.

### EXAMPLE 14

### 5-{4-[(4-Chlorobenzyl)amino]phenyl}-6-{[(2-fluorobenzyl)oxy]methyl}pyrimidine-2,4-diamine

The titled compound was prepared according to the procedure described in Example 10, substituting 2-fluorobenzyl bromide for 1-(bromomethyl)-3-methylbenzene used in Example 10. ¹H NMR (500 MHz, DMSO-d₆) δ ppm 3.96 (s, 2 H), 4.27 (d, J=5.61 Hz, 2 H), 4.39 (s, 2 H), 5.86 (s, 2 H), 6.34 (t, J=5.93 Hz, 1 H), 6.59 (d, J=8.73 Hz, 2 H), 6.89 (d, J=8.42 Hz, 2 H), 7.11 (m, 2 H), 7.29 (m, 2 H), 7.40 (m, 4 H); MS (ESI) positive ion 464(M+H)⁺.

### EXAMPLE 15

### 5-{4-[(4-Chlorobenzyl)amino]phenyl}-6-{[(4-fluorobenzyl)oxy]methyl}pyrimidine-2,4-diamine

The titled compound was prepared according to the procedure described in Example 10, substituting 4-fluorobenzyl bromide for 1-(bromomethyl)-3-methylbenzene used in Example 10. ¹H NMR (500 MHz, DMSO-d₆) δ ppm 3.93 (s, 2 H), 4.27 (d, J=5.93 Hz, 2 H), 4.30 (s, 2 H), 5.85 (s, 2 H), 6.35 (t, J=5.93 Hz, 1 H), 6.60 (d, J=8.42 Hz, 2 H), 6.88 (d, J=8.42 Hz, 2 H), 7.07 (t, J=8.89 Hz, 2 H), 7.20 (dd, J=8.42, 5.61 Hz, 2 H), 7.39 (m, 4 H); MS (ESI) positive ion 464(M+H)⁺.

### EXAMPLE 16

### 5-{4-[(4-Chlorobenzyl)amino]phenyl}-6-{[(2-chlorobenzyl)oxy]methyl}pyrimidine-2,4-diamine

The titled compound was prepared according to the procedure described in Example 10, substituting 2-chlorobenzyl bromide for 1-(bromomethyl)-3-methylbenzene used in Example 10. ¹H NMR (500 MHz, DMSO-d₆) δ ppm 4.01 (s, 2 H), 4.27 (d, J=5.93 Hz, 2 H), 4.41 (s, 2 H), 5.51 (m, 2 H), 5.87 (s, 2 H), 6.35 (t, J=5.93 Hz, 1 H), 6.59 (d, J=8.42 Hz, 2 H), 6.90 (d, J=8.42 Hz, 2 H), 7.27 (m, 3 H), 7.39 (m, 5 H); MS (ESI) positive ion 481 (M+H)⁺.

### EXAMPLE 17

### 5-{4-[(4-Chlorobenzyl)amino]phenyl}-6-{[(4-chlorobenzyl)oxy]methyl}pyrimidine-2,4-diamine

The titled compound was prepared according to the procedure described in Example 10, substituting 4-chlorobenzyl bromide for 1-(bromomethyl)-3-methylbenzene used in Example 10. ¹H NMR (500 MHz, DMSO-d₆) δ ppm 3.94 (s, 2 H), 4.27 (d, J=5.93 Hz, 2 H), 4.32 (s, 2 H), 5.85 (s, 2 H), 6.35 (t, J=6.08 Hz, 1 H), 6.60 (d, J=8.73 Hz, 2 H), 6.88 (d, J=8.42 Hz, 2 H), 7.18 (d, J=8.42 Hz, 2 H), 7.32 (d, J=8.42 Hz, 2 H), 7.40 (m, 4 H); MS (ESI) positive ion 481(M+H)⁺.

### EXAMPLE 18

### 6-{[(2-Bromobenzyl)oxy]methyl}-5-{4-[(4-chlorobenzyl)amino]phenyl}pyrimidine-2,4-diamine

The titled compound was prepared according to the procedure described in Example 10, substituting 2-bromobenzyl bromide for 1-(bromomethyl)-3-methylbenzene used in Example 10. ¹H NMR (500 MHz, DMSO-d₆) δ ppm 4.02 (s, 2 H), 4.27 (d, J=5.93 Hz, 2 H), 4.38 (s, 2 H), 5.89 (s, 2 H), 6.35 (t, J=6.08 Hz, 1 H), 6.59 (d, J=8.73 Hz, 2 H), 6.90 (d, J=8.42 Hz, 2 H), 7.20 (m, 1 H), 7.28 (m, 2 H), 7.39 (m, 4 H), 7.55 (d, J=7.49 Hz, 1 H); MS (ESI) positive ion 525(M+H)⁺.

### EXAMPLE 19

### 5-{4-[(4-Chlorobenzyl)amino]phenyl}-6-({[3-(trifluoromethyl)benzyl]oxy}methyl)pyrimidine-2,4-diamine

The titled compound was prepared according to the procedure described in Example 10, substituting 3-1′,1′,1′-trifluoromethylbenzyl bromide for 1-(bromomethyl)-3-methylbenzene used in Example 10. ¹H NMR (500 MHz, DMSO-d₆) δ ppm 4.00 (s, 2 H), 4.26 (d, J=5.93 Hz, 2 H), 4.43 (s, 2 H), 5.89 (m, 2 H), 6.35 (t, J=6.08 Hz, 1 H), 6.59 (d, J=8.42 Hz, 2 H), 6.88 (d, J=8.42 Hz, 2 H), 7.39 (m, 4 H), 7.49 (m, 2 H), 7.58 (m, 2 H); MS (ESI) positive ion 514(M+H)⁺.

### EXAMPLE 20

### 5-{4-[(4-Chlorobenzyl)amino]phenyl}-6-({[4-(methylthio)benzyl]oxy}methyl)pyrimidine-2,4-diamine

The titled compound was prepared according to the procedure described in Example 10, substituting 1-bromomethyl-4-methylsulfanyl-benzene for 1-(bromomethyl)-3-methylbenzene used in Example 10. ¹H NMR (500 MHz, DMSO-d₆) δ ppm 3.92 (s, 2 H), 4.28 (m, 4 H), 5.43 (m, 2 H), 5.84 (s, 2 H), 6.34 (t, J=5.93 Hz, 1 H), 6.60 (d, J=8.42 Hz, 2 H), 6.89 (d, J=8.42 Hz, 2 H), 7.11 (d, J=8.42 Hz, 2 H), 7.17 (m, 2 H), 7.40 (m, 5 H); MS (ESI) positive ion 492(M+H)⁺.

### EXAMPLE 21

### 5-{4-[(4-Chlorobenzyl)amino]phenyl}-6-{[(2,4-dimethylbenzyl)oxy]methyl}pyrimidine-2,4-diamine

The titled compound was prepared according to the procedure described in Example 10, substituting 1-bromomethyl-2,4-dimethyl-benzene for 1-(bromomethyl)-3-methylbenzene used in Example 10. ¹H NMR (500 MHz, DMSO-d₆) δ ppm 2.12 (s, 3 H), 2.23 (s, 3 H), 3.93 (s, 2 H), 4.27 (m, 4 H), 5.45 (s, 2 H), 5.83 (s, 2 H), 6.35 (t, J=5.77 Hz, 1 H), 6.59 (d, J=8.42 Hz, 2 H), 6.88 (d, J=8.42 Hz, 3 H), 6.94 (m, 1 H), 7.00 (d, J=7.80 Hz, 1 H), 7.40 (m, 4 H); MS (ESI) positive ion 474(M+H)⁺.

### EXAMPLE 22

### 5-{4-[(4-Chlorobenzyl)amino]phenyl}-6-{[(3,5-dimethylbenzyl)oxy]methyl}pyrimidine-2,4-diamine

The titled compound was prepared according to the procedure described in Example 10, substituting 1-bromomethyl-3,5-dimethyl-benzene for 1-(bromomethyl)-3-methylbenzene used in Example 10. ¹H NMR (500 MHz, DMSO-d₆) δ ppm 2.22 (s, 6 H), 3.92 (s, 2 H), 4.24 (s, 2 H), 4.27 (d, J=5.93 Hz, 2 H), 5.46 (s, 2 H), 5.85 (s, 2 H), 6.34 (t, J=5.93 Hz, 1 H), 6.60 (d, J=8.42 Hz, 2 H), 6.78 (s, 2 H), 6.86 (s, 1 H), 6.89 (d, J=8.73 Hz, 2 H), 7.38 (m, 4 H); MS (ESI) positive ion 474(M+H)⁺.

### EXAMPLE 23

### 5-{4-[(4-Chlorobenzyl)amino]phenyl}-6-{[(2,3-dichlorobenzyl)oxy]methyl}pyrimidine-2,4-diamine

The titled compound was prepared according to the procedure described in Example 10, substituting 2,3-dichlorbenzyl bromide for 1-(bromomethyl)-3-methylbenzene used in Example 10. ¹H NMR (500 MHz, DMSO-d₆) δ ppm 4.02 (s, 2 H), 4.26 (d, J=5.93 Hz, 2 H), 4.45 (s, 2 H), 5.50 (s, 2 H), 5.87 (s, 2 H), 6.35 (t, J=6.08 Hz, 1 H), 6.58 (d, J=8.42 Hz, 2 H), 6.88 (d, J=8.42 Hz, 1 H), 7.26 (m, 2 H), 7.38 (m, 5 H), 7.53 (dd, J=7.17, 2.50 Hz, 1 H); MS (ESI) positive ion 516(M+H)⁺.

### EXAMPLE 24

### 5-{4-[(4-Chlorobenzyl)amino]phenyl}-6-{[(2,5-dichlorobenzyl)oxy]methyl}pyrimidine-2,4-diamine

The titled compound was prepared according to the procedure described in Example 10, substituting 2,5-dichlorbenzyl bromide for 1-(bromomethyl)-3-methylbenzene used in Example 10. ¹H NMR (500 MHz, DMSO-d₆) δ ppm 4.05 (s, 2 H), 4.26 (d, J=5.61 Hz, 2 H), 4.40 (s, 2 H), 5.53 (s, 2 H), 5.89 (s, 2 H), 6.34 (t, J=5.93 Hz, 1 H), 6.59 (d, J=8.73 Hz, 2 H), 6.88 (d, J=8.42 Hz, 2 H), 7.39 (m, 7 H); MS (ESI) positive ion 516(M+H)⁺.

### EXAMPLE 25

### 5-{4-[(1,3-Benzodioxol-4-ylmethyl)amino]phenyl}-6-[(benzyloxy)methyl]pyrimidine-2,4-diamine

The title compound was synthesized according to the procedure described in Example 2, substituting 2.3-(methylenedioxy)benzaldehyde for 4-chlorobenzaldehyde. ¹H NMR (300 MHz, DMSO-d₆) δ 3.95 (s, 2 H), 4.21 (d, J=5.76 Hz, 2 H), 4.33 (s, 2 H), 5.50 (s, 2 H), 5.88 (s, 2 H), 6.04 (s, 2 H), 6.22 (t, J=5.93 Hz, 1 H), 6.63 (d, J=8.48 Hz, 2 H), 6.81 (m, 2 H), 6.89 (m, 1 H), 6.91 (d, J=8.48 Hz, 2 H), 7.19 (m, 2 H), and 7.27 (m, 3 H). MS (ESI) positive ion 456 (M+H)⁺; negative ion 454 (M−H)⁻.

### EXAMPLE 26

### tert-butyl 2-[(4-{2,4-Diamino-6-[(benzyloxy)methyl]pyrimidin-5-yl}phenyl)amino]ethylcarbamate

The title compound was synthesized according to the procedure described in Example 2, substituting tert-butyl N-(2-oxoethyl)carbamate for 4-chlorobenzaldehyde. ¹H NMR (300 MHz, DMSO-d₆) δ 1.39 (s, 9 H), 3.09 (m, 4 H), 4.03 (s, 2 H), 4.38 (s, 2 H), 5.76 (s, 2 H), 6.24 (s, 2 H), 6.61 (d, J=8.48 Hz, 2 H), 6.90 (m, 1 H), 6.92 (d, J=8.48 Hz, 2 H), 7.23 (m, 2 H), and 7.30 (m, 3 H). MS (ESI) positive ion 465 (M+H)⁺; negative ion 463 (M−H)⁻ and 389 (M−75)⁻.

### EXAMPLE 27

### 6-[(Benzyloxy)methyl]-5-{4-[(3-furylmethyl)amino]phenyl}pyrimidine-2,4-diamine

The title compound was synthesized according to the procedure described in Example 2, substituting 2-furaldehyde for 4-chlorobenzaldehyde. ¹H NMR (300 MHz, DMSO-d₆) δ 3.96 (s, 2 H), 4.26 (d, J=6.10 Hz, 2 H), 4.34 (s, 2 H), 5.54 (s, 2 H), 5.91 (s, 2 H), 6.19 (t, J=5.93 Hz, 1 H), 6.33 (dd, J=3.22, 0.85 Hz, 1 H), 6.39 (m, 1 H), 6.69 (d, J=8.82 Hz, 2 H), 6.92 (d, J=8.48 Hz, 2 H), 7.19 (m, 2 H), 7.28 (m, 3 H), and 7.58 (dd, J=1.86, 0.85 Hz, 1 H). MS (ESI) positive ion 402 (M+H)⁺; negative ion 400 (M−H)⁻.

### EXAMPLE 28

### 6-[(Benzyloxy)methyl]-5-{4-[(tetrahydrofuran-3-ylmethyl)amino]phenyl}pyrimidine-2,4-diamine

The title compound was synthesized according to the procedure described in Example 2, substituting tetrahydrofuran-3-carboxaldehyde for 4-chlorobenzaldehyde. ¹H NMR (300 MHz, DMSO-d₆) δ 1.61 (m, 1 H), 2.01 (m, 1 H), 2.48 (m, 1 H), 3.00 (m, 2 H), 3.47 (dd, J=8.48, 5.42 Hz, 1 H), 3.64 (m, 1 H), 3.77 (m, 2 H), 3.97 (s, 2 H), 4.35 (s, 2 H), 5.55 (s, 2 H), 5.81 (t, J=5.59 Hz, 1 H), 5.90 (s, 2 H), 6.61 (d, J=8.48 Hz, 2 H), 6.91 (d, J=8.48 Hz, 2 H), 7.20 (m, 2 H), and 7.28 (m, 3 H). MS (ESI) positive ion 406 (M+H)⁺; negative ion 404 (M−H)⁻.

### EXAMPLE 29

### 4-Chloro-N-(4-{2,4-diamino-6-[(benzyloxy)methyl]pyrimidin-5-yl}phenyl)benzamide

5-(4-Amino-phenyl)-6-benzyloxymethyl-pyrimidine-2,4-diamine (50 mg, 0.16 mmol) from example 2C, 4-chlorobenzoic acid (24 mg, 0.16 mmol), and TBTU (70 mg, 0.22 mmol) were dissolved in DMF (1 mL). The mixture was stirred for 5 minutes followed by the addition of Et₃N (0.27 mL, 1.6 mmol). The reaction mixture was stirred for 2 hours at room temperature and separated by reverse phase HPLC (0-70% CH₃CN in aq. NH₄OAc) providing 4-chloro-N-[4-(2,4-diamino-6-benzyloxymethyl-pyrimidin-5-yl)-phenyl]-benzamide (25 mg, 35%) as an off-white solid. ¹H NMR (300 MHz, DMSO-d₆) δ 3.99 (s, 2 H), 4.34 (s, 2 H), 5.65 (s, 2 H), 5.99 (s, 2 H), 7.20 (m, 4 H), 7.28 (m, 3 H), 7.63 (m, 2 H), 7.83 (d, J=8.48 Hz, 2 H), 8.00 (ddd, J=8.99, 2.37, 2.20 Hz, 2 H), and 10.38 (s, 1 H). MS (ESI) positive ion 460 (M+H)⁺; negative ion 458 (M−H)⁻.

### EXAMPLE 30

### 6-[(Benzyloxy)methyl]-5-{4-[(pyridin-2-ylmethyl)amino]phenyl}pyrimidine-2,4-diamine

The title compound was synthesized according to the procedure described in Example 2, substituting 2-pyridinecarboxaldehyde for 4-chlorobenzaldehyde. ¹H NMR (300 MHz, DMSO-d₆) δ 3.94 (s, 2 H), 4.32 (s, 2 H), 4.37 (d, J=6.10 Hz, 2 H), 5.48 (s, 2 H), 5.87 (s, 2 H), 6.42 (t, J=5.93 Hz, 1 H), 6.62 (d, J=8.48 Hz, 2 H), 6.90 (d, J=8.48 Hz, 2 H), 7.18 (m, 2 H), 7.25 (m, 4 H), 7.41 (d, J=7.80 Hz, 1 H), 7.74 (td, J=7.63, 2.03 Hz, 1 H), and 8.54 (ddd, J=4.83, 1.78, 0.85 Hz, 1 H). MS (ESI) positive ion 413 (M+H)⁺; 411 (M−H)⁻.

### EXAMPLE 31

### 6-[(Benzyloxy)methyl]-5-{4-[(pyridin-3-ylmethyl)amino]phenyl}pyrimidine-2,4-diamine

The title compound was synthesized according to the procedure described in Example 2, substituting 3-pyridinecarboxaldehyde for 4-chlorobenzaldehyde. ¹H NMR (300 MHz, DMSO-d₆) δ 3.94 (s, 2 H), 4.32 (d, 5.81 Hz, 2 H), 4.32 (s, 2 H), 5.50 (s, 2 H), 5.88 (s, 2 H), 6.36 (t, J=5.93 Hz, 1 H), 6.64 (d, J=8.48 Hz, 2 H), 6.91 (d, J=8.82 Hz, 2 H), 7.18 (m, 2 H), 7.26 (m, 3 H), 7.36 (ddd, J=7.80,4.75, 0.68 Hz, 1 H), 7.79 (dt, J=7.80, 1.87 Hz, 1 H), 8.46 (dd, J=4.92, 1.53 Hz, 1 H), and 8.62 (d, J=2.37 Hz, 1 H). MS (ESI) positive ion 413 (M+H)⁺.

### EXAMPLE 32

### 6-[(Benzyloxy)methyl]-5-{4-[(1H-imidazol-4-ylmethyl)amino]phenyl}pyrimidine-2,4-diamine

The title compound was synthesized according to the procedure described in Example 2, substituting 4(5)imidazolecarboxaldehyde for 4-chlorobenzaldehyde. ¹H NMR (300 MHz, DMSO-d₆) δ 3.96 (s, 2 H), 4.15 (d, J=3.73 Hz, 2 H), 4.34 (s, 2 H), 5.49 (s, 2 H), 5.88 (s, 2 H), 5.90 (t, J=5.34 Hz, 1 H), 6.69 (d, J=8.82 Hz, 2 H), 6.91 (d, J=8.48 Hz, 2 H), 6.97 (s, 1 H), 7.20 (m, 2 H), 7.28 (m, 3 H), and 7.57 (d, J=1.02 Hz, 1 H). MS (ESI) positive ion 402 (M+H)⁺; negative ion 400 (M−H)⁻.

### EXAMPLE 33

### 6-[(Benzyloxy)methyl]-5-[4-(dimethylamino)phenyl]pyrimidine-2,4-diamine

The title compound was synthesized according to the procedure described in Example 2, substituting excess formaldehyde for 4-chlorobenzaldehyde. ¹H NMR (300 MHz, DMSO-d₆) δ 2.93 (s, 6 H), 3.97 (s, 2 H), 4.35 (s, 2 H), 5.56 (s, 2 H), 5.93 (s, 2 H), 6.75 (d, J=8.82 Hz, 2 H), 7.02 (d, J=8.82 Hz, 2 H), 7.20 (m, 2 H), and 7.28 (m, 3 H). MS (ESI) positive ion 350 (M+H)⁺; negative ion 348 (M−H)⁻.

### EXAMPLE 34

### 6-[(Benzyloxy)methyl]-5-[4-(methylamino)phenyl]pyrimidine-2,4-diamine

The title compound was synthesized according to the procedure described in Example 2, substituting formaldehyde for 4-chlorobenzaldehyde. ¹H NMR (300 MHz, DMSO-d₆) δ 2.70 (d, J=4.75 Hz, 3 H), 3.97 (s, 2 H), 4.35 (s, 2 H), 5.51 (s, 2 H), 5.71 (q, J=5.09 Hz, 1 H), 5.88 (s, 2 H), 6.57 (d, J=8.81 Hz, 2 H), 6.93 (d, J=8.48 Hz, 2 H), 7.21 (m, 2 H), and 7.28 (m, 3 H). MS (ESI) positive ion 336 (M+H)⁺.

### EXAMPLE 35

### 6-[(Benzyloxy)methyl]-5-[4-(ethylamino)phenyl]pyrimidine-2,4-diamine

The title compound was synthesized according to the procedure described in Example 2, substituting acetaldehyde for 4-chlorobenzaldehyde. ¹H NMR (300 MHz, DMSO-d₆) δ 1.18 (t, J=7.12 Hz, 3 H), 3.05 (m, 2 H), 3.97 (s, 2 H), 4.35 (s, 2 H), 5.54 (s, 2 H), 5.61 (m, 1 H), 5.90 (s, 2 H), 6.58 (d, J=8.48 Hz, 2 H), 6.91 (d, J=8.48 Hz, 2 H), 7.21 (m, 2 H), and 7.28 (m, 3 H). MS (ESI) 350 positive ion (M+H)⁺.

### EXAMPLE 36

### 6-[(Benzyloxy)methyl]-5-[4-(propylamino)phenyl]pyrimidine-2,4-diamine

The title compound was synthesized according to the procedure described in Example 2, substituting propionaldehyde for 4-chlorobenzaldehyde. ¹H NMR (300 MHz, DMSO-d₆) δ 0.96 (t, J=7.29 Hz, 3 H), 1.58 (sextet, J=7.12 Hz, 2 H), 2.98 (m, 2 H), 3.97 (s, 2 H), 4.35 (s, 2 H), 5.49 (s, 2 H), 5.66 (t, J=5.43 Hz, 1 H), 5.86 (s, 2 H), 6.59 (d, J=8.48 Hz, 2 H), 6.90 (d, J=8.48 Hz, 2 H), 7.20 (m, 2 H), and 7.28 (m, 3 H). MS (ESI) positive ion 364 (M+H)⁺; negative ion 362 (M−H)⁻.

### EXAMPLE 37

### 6-[(Benzyloxy)methyl]-5-[4-(isobutylamino)phenyl]pyrimidine-2,4-diamine

The title compound was synthesized according to the procedure described in Example 2, substituting isobutyraldehyde for 4-chlorobenzaldehyde. ¹H NMR (300 MHz, DMSO-d₆) δ 0.95 (d, J=6.44 Hz, 6 H), 1.85 (m, 1 H), 2.83 (t, J=6.27 Hz, 2 H), 3.97 (s, 2 H), 4.35 (s, 2 H), 5.50 (s, 2 H), 5.71 (t, J=5.76 Hz, 1 H), 5.86 (s, 2 H), 6.59 (d, J=8.48 Hz, 2 H), 6.90 (d, J=8.48 Hz, 2 H), 7.20 (m, 2 H), and 7.27 (m, 3 H). MS (ESI) positive ion 378 (M+H)⁺.

### EXAMPLE 38

### 6-[(Benzyloxy)methyl]-5-[4-(neopentylamino)phenyl]pyrimidine-2,4-diamine

The title compound was synthesized according to the procedure described in Example 2, substituting trimethylacetaldehyde for 4-chlorobenzaldehyde. ¹H NMR (300 MHz, DMSO-d₆) δ 0.97 (s, 9 H), 2.83 (d, J=5.76 Hz, 2 H), 4.03 .(s, 2 H), 4.38 (s, 2 H), 5.57 (t, J=5.76 Hz, 1 H), 6.04 (s, 2 H), 6.29 (s, 2 H), 6.68 (d, J=8.48 Hz, 2 H), 6.89 (d, J=8.48 Hz, 2 H), 7.22 (m, 2 H), and 7.30 (m, 3 H). MS (ESI) positive ion 392 (M+H)⁺; negative ion 390 (M−H)⁻.

### EXAMPLE 39

### 6-[(Benzyloxy)methyl]-5-{4-[(cyclopropylmethyl)amino]phenyl}pyrimidine-2,4-diamine

The title compound was synthesized according to the procedure described in Example 2, substituting cycloproylcarboxaldehyde for 4-chlorobenzaldehyde. ¹H NMR (300 MHz, DMSO-d₆) δ 0.22 (ddd, J=5.85, 4.49, 4.24 Hz, 2 H), 0.48 (ddd, J=8.05, 5.85, 4.07 Hz, 2 H), 1.06 (m, 1 H), 2.90 (t, J=5.93 Hz, 2 H), 3.98 (s, 2 H), 4.35 (s, 2 H), 5.56 (s, 2 H), 5.74 (t, J=5.43 Hz, 1 H), 5.93 (s, 2 H), 6.61 (d, J=8.48 Hz, 2 H), 6.91 (d, J=8.48 Hz, 2 H), 7.21 (m, 2 H), and 7.29 (m, 3 H). MS (ESI) positive ion 376 (M+H)⁺; negative ion 374 (M−H)⁻.

### EXAMPLE 40

### 2-Butoxy-N-(4-{2,4-diamino-6-[(benzyloxy)methyl]pyrimidin-5-yl}phenyl)acetamide

The title compound was synthesized according to the procedure described in Example 29, substituting n-butoxyacetic acid for 4-chlorobenzoic acid. ¹H NMR (300 MHz, DMSO-d₆) δ 0.91 (t, J=7.29 Hz, 3 H) 1.37 (m, 2 H) 1.58 (m, 2 H) 3.52 (t, J=6.61 Hz, 2 H) 3.98 (s, 2 H) 4.05 (s, 2 H) 4.34 (s, 2 H) 5.81 (s, 2 H) 6.12 (s, 2 H) 7.15 (d, J=8.48 Hz, 2 H) 7.16 (m, 2 H) 7.26 (m, 3 H) 7.70 (d, J=8.81 Hz, 2 H) 9.75 (s, 1 H). MS (ESI) positive ion 436 (M+H)⁺; negative ion 434 (M−H)⁻.

### EXAMPLE 41

### 5-{4-[(4-Chlorobenzyl)amino]phenyl}-6-tetrahydrofuran-2-ylpyrimidine-2,4-diamine

### EXAMPLE 41 A

### 5-(4-Amino-phenyl)-6-(tetrahydro-furan-2-yl)-pyrimidine-2,4-diamine

The titled compound was prepared according to the procedure described in Example 2, substituting tetrahydrofuran-2-carbonyl chloride for benzyloxyacetyl chloride used in Example 2.

### EXAMPLE 41B

### 5-{4-[(4-Chlorobenzyl)amino]phenyl}-6-tetrahydrofuran-2-ylpyrimidine-2,4-diamine

To a stirred solution of 5-(4-amino-phenyl)-6-(tetrahydrofuran-2-yl)-pyrimidine-2,4-diamine (40 mg, 0.147 mmol) in MeOH (1.5 mL) was added 4-chlorobenzaldehyde (20 mg, 0.147 mmol). After mixture was stirred for 30 minutes at room temperature then cooled to 0° C. Glacial acetic acid (0.03 mL, 0.53 mmol) was added followed by NaCNBH₃ (10 mg, 0.162 mmol). The reaction warmed to room temperature over 2 hours and the solvent removed under reduced pressure. The residue was taken up in aqueous NaHCO₃ (5 mL) washed with EtOAc (2×8 mL) and the combined organic layers washed with brine (10 mL), dried over MgSO₄, filtered and concentrated under reduced pressure. The solid was triturated from Et₂O and filtered to provide a pale yellow solid (10 mg, 17%). ¹H NMR (300 MHz, DMSO-d₆) δ 7.37-7.43 (m, 4H), 6.84 (d, J=14.24 Hz, 2H), 6.61 (d, J=8.14 Hz, 2H), 6.35 (t, J=6.10 Hz, 1H), 5.79 (s, 2H), 5.42 (s, 2H), 4.26 (d, J=6.10 Hz, 2H), 3.79 (q, J=6.89 Hz, 1 H), 3.54-3.61 (m, 1H), 1.96-2.01 (m, 2H), 1.66-1.84 (m, 2H). MS (ESI) positive ion 396 (M+H)⁺; negative ion 394 (M−H)⁻.

### EXAMPLE 42

### 6-[(2-Butoxyethoxy)methyl]-5-{4-[(4-chlorobenzyl)amino]phenyl}pyrimidine-2,4-diamine

### EXAMPLE 42A

### 5-(4-Amino-phenyl)-6-(2-butoxy-ethoxymethyl)-pyrimidine-2,4-diamine

The titled compound was prepared according to the procedure described in Example 2, substituting tetrahydrofuran-2-carbonyl chloride for benzyloxyacetyl chloride used in Example 2.

### EXAMPLE 42B

### 6-[(2-Butoxyethoxy)methyl]-5-{4-[(4-chlorobenzyl)amino]phenyl}pyrimidine-2,4-diamine

To a stirred solution of 5-(4-amino-phenyl)-6-(2-butoxy-ethoxymethyl)-pyrimidine-2,4-diamine (140 mg, 0.536 mmol) in MeOH (5.3 mL) was added 4-chlorobenzaldehyde (75 mg, 0.536 mmol). After 30 minutes at room temperature, the reaction was cooled to 0° C. Glacial acetic acid (0.1 mL, 1.5 mmol) was added followed by NaCNBH₃ (37 mg, 0.588 mmol). The mixture was warmed to room temperature over 2 hours, the solvent was removed under reduced pressure and the residue taken up in saturated NaHCO₃ (10 mL). The solution was washed with EtOAc (2×10 mL) and the combined organic layers washed with brine (10 mL), dried over MgSO₄, filtered and concentrated under reduced pressure. Purification on reverse-phase HPLC (0-70% CH₃CN, aqueous NH₄OAc) provided a white powder (15 mg, 6 %). ¹H NMR (300 MHz, DMSO-d₆) δ 7.36-7.43 (m, 4H), 6.89 (d, J=8.48 Hz, 2H), 6.60 (d, J=8.48 Hz, 2H), 6.40 (t, J=5.93 Hz, 1H), 6.10 (s, 2H), 5.78 (s, 2H), 4.27 (d, J=5.76 Hz, 2H), 3.93 (s, 2H), 3.35-3.39 (m, 4 H), 3.29-3.31 (m, 2H), 1.37-1.46 (m, 2H), 1.20-1.32 (m, 2H), 0.845 (t, J=7.29 Hz, 3 H). MS (ESI) positive ion 456 (M+H)⁺; negative ion 454 (M−H)⁻.

### EXAMPLE 43

### 6-[(Benzyloxy)methyl]-5-{4-[(1-ethylpropyl)amino]phenyl}pyrimidine-2,4-diamine

The title compound was synthesized according to the procedure described in Example 2, substituting 3-pentanone for 4-chlorobenzaldehyde. ¹H NMR (300 MHz, DMSO-d₆) δ 0.90 (t, J=7.29 Hz, 6 H), 1.49 (m, 4 H), 3.17 (m, 1 H), 3.98 (s, 2 H), 4.35 (s, 2 H), 5.42 (d, J=8.14 Hz, 1 H), 5.50 (s, 2 H), 5.86 (s, 2 H), 6.58 (d, J=8.48 Hz, 2 H), 6.88 (d, J=8.48 Hz, 2 H), 7.20 (m, 2 H), and 7.27 (m, 3 H). MS (ESI) positive ion 392 (M+H)⁺; negative ion 390 (M−H)⁻.

### EXAMPLE 44

### 4-{[(4-{2,4-Diamino-6-[(benzyloxy)methyl]pyrimidin-5yl}phenyl)amino]methyl}benzonitrile

The titled compound was prepared according to the procedure described in Example 2, substituting 4-cyano-benzaldehyde for 4-chloro-benzaldehyde. ¹H NMR (300 MHz, DMSO-d₆) δ 7.80 (d, J=8.1 Hz, 2H), 7.59 (d, J=8.5 Hz, 2H), 7.28-7.15 (m, 5H), 6.90 (d, J=8.5 Hz, 2H), 6.59 (d, J=8.5 Hz, 2H), 6.48 (t, J=6.1 Hz, 1H), 5.87 (s, 2H), 5.47 (bs, 2H), 4.39 (d, J=6.1 Hz, 2H), 4.31 (s, 2H), 3.93 (s, 2H). MS (ESI) positive ion 437 (M+H)⁺; negative ion 435 (M−H)⁻.

### EXAMPLE 45

### 4-{[(4-{2,4-Diamino-6-[(benzyloxy)methyl]pyrimidin-5-yl}phenyl)(methyl)amino]methyl}benzonitrile

NaBH₃CN (5 mg, 0.08 mmol) was added to a mixture of 4-{[(4-{2,4-diamino-6-[(benzyloxy)methyl]pyrimidin-5-yl}phenyl)amino]methyl}benzonitrile from Example 44 (22 mg, 0.05 mmol), 37% formaldehyde (5 μL, 0.06 mmol) and acetic acid (5 μL) in methanol (1 mL). The mixture was stirred at room temperature for 2 hours after which another portion of acetic acid (5 μL), formaldehyde (5 μL, 0.08 mmol), and NaBH₃CN (5 mg, 0.06 mmol) was added and stirred for another hour. The mixture was partitioned between ethyl acetate and aqueous NaHCO₃ (20 mL, 1:1). The organic phase was washed with brine, dried (MgSO₄), filtered and concentrated under reduced pressure. The residue was purified on silica gel with ethyl acetate/methanol (10/1) to provide the titled compound (15 mg). ¹H NMR (300 MHz, DMSO-d₆) δ 7.79 (d, J=8.5 Hz, 2H), 7.43 (d, J=8.1 Hz, 2H), 7.30-7.15 (m, 5H), 7.00 (d, J=8.8 Hz, 2H), 6.73 (d, J=8.8 Hz, 2H), 5.89 (s, 2H), 5.51 (bs, 2H), 4.68 (s, 2H), 4.32 (s, 2H), 3.95 (s, 2H), 3.06 (s, 3H). MS (ESI) positive ion 451 (M+H)⁺; negative ion 449 (M−H)⁻.

### EXAMPLE 46

### 5-{4-[(4-Chlorobenzyl)amino]phenyl}-6-[(3-methylbutoxy)methyl]pyrimidine-2,4-diamine

### EXAMPLE 46A

### 3-(Methylbutoxy)acetic acid

Allyl isoamyl glycolate (0.5 g, 2.68 mmol) was dissolved in MeOH (6 mL) and 2 M NaOH (6 mL) was added. After 1 hour, the mixture was concentrated under reduced pressure and the remainder acidified with 1 M HCl to pH 3, The solution was extracted with EtOAc (3×10 mL) and the combined organic layers washed with brine, dried over MgSO₄ filtered and concentrated to provide the title compound as a clear oil (371 mg, 95%).

### EXAMPLE 46B

### 3-(Methylbutoxy)acetyl chloride

3-(Methylbutoxy)acetic acid (1.6 g, 10.9 mmol) was dissolved in SOCl₂ (8 mL, 100 mmol) and heated to reflux for 3 hours. The mixture was cooled to room temperature and concentrated under reduced pressure. The resulting acid chloride was taken on to the next step without further purification.

### EXAMPLE 46C

### 4-(3-Methylbutoxy)-2-(4-nitrophenyl)-3-oxo-butyronitrile

To a solution of 4-nitrophenylacetonitrile (500 mg, 3.0 mmol) in CH₂Cl₂ (5 mL) at 0° C. was added Et₃N (0.86 mL, 6.0 mmol) and DMAP (38 mg, 0.3 mmol). A solution of 3-(methylbutoxy)acetyl chloride (10 mmol) from Example 46B in CH₂Cl₂ (2 mL) was slowly added. The reaction was warmed to room temperature and stirred for 1 hour. The mixture was diluted with EtOAc (20 mL) and washed with 1 M HCl (10 mL), brine (10 mL), dried over MgSO₄, filtered and concentrated to provide the titled compound as a dark green solid (580 mg, 66%).

### EXAMPLE 46D

### 6-(3-Methylbutoxymethyl)-5-(4-nitrophenyl)-pyrimidine-2,4-diamine

To a solution of 4-(3-methylbutoxy)-2-(4-nitrophenyl)-3-oxo-butyronitrile (580 mg, 2.0 mmol) from Example 46C in CH₂Cl₂ (4.5 mL) and MeOH (0.5 mL) at 0° C. was added trimethylsilyl-diazomethane (2.0 M in Et₂O, 3 mL, 6.0 mmol). The reaction was stirred at room temperature for 1 hour. Glacial acetic acid (3 mL) was slowly added to quench excess TMS-diazomethane. The mixture was diluted with EtOAc (20 mL) and washed with aqueous NaHCO₃ solution (2×10 mL), brine (10 mL), dried over Mg SO₄, filtered and concentrated under reduced pressure. The residue was taken up in EtOH (10 mL) followed by the addition of guanidine HCl (190 mg, 2.0 mmol) in EtOH (2 mL) and KOEt (1.0 mL, 2.0 mmol). The mixture was heated to refluxed for 1 hour after which it was concentrated under reduced pressure, taken up in 2M NaOH (30 mL) and filtered. The resulting black solid was recrystallized from EtOH to provide the titled compound as a yellow solid (160 mg, 24%).

### EXAMPLE 46E

### 5-(4-Aminophenyl)-6-(3-methylbutoxymethyl)-pyrimidine-2,4-diamine

To a flask containing 6-(3-methylbutoxymethyl)-5-(4-nitrophenyl)-pyrimidine-2,4-diamine (150 mg, 0.453 mmol) from Example 46D was added 10% Pd/C (15 mg, 0.014 mmol) and glacial acetic acid (4.5 mL). The mixture was placed under an atmosphere of H₂ and stirred at room temperature for 4 hours. The mixture was filtered through Celite and concentrated under reduced pressure to provide the title compound as a clear yellow oil (125 mg, 92%).

### EXAMPLE 46F

### 5-{4-[(4-Chlorobenzyl)amino]phenyl}-6-[(3-methylbutoxy)methyl]pyrimidine-2,4-diamine

To a solution of 5-(4-aminophenyl)-6-(3-methylbutoxymethyl)-pyrimidine-2,4-diamine from Example 46E (120 mg, 0.40 mmol) in MeOH (4 mL) was added 4-chlorobenzaldehyde (56 mg, 0.40 mmol). The mixture was stirred for 30 minutes at room temperature then cooled to 0° C. Glacial acetic acid (0.06 mL, 1.0 mmol) was added followed by NaCNBH₃ (28 mg, 0.44 mmol). The mixture was warmed to room temperature over 1 hour after which aqueous NaHCO₃ (8 mL) was added to the reaction. The mixture was extracted with EtOAc (2×15 mL) and the combined organic layers washed with brine (10 mL), dried over MgSO₄, filtered and concentrated. Purification on reverse-phase HPLC (0-70% CH₃CN, aqueous NH₄OAc) provided the titled compound as an off white powder (20 mg, 12%). ¹H NMR (300 MHz, DMSO-d₆) δ 7.35-7.41 (m, 4H), 6.88 (d, J=8.48 Hz, 2H), 6.60 (d, J=8.48 Hz, 2H), 6.35 (t, J=5.98 Hz, 1H), 5.95 (s, 2H), 5.62 (s, 2H), 4.26 (d, J=5.83 Hz, 2H), 3.83 (s, 2H), 3.21 (t, J=6.75 Hz, 2H), 1.50-1.57 (m, 1H), 1.24 (q, J=6.44 Hz, 2H), 0.774 (d, J=6.0 Hz, 6 H). MS (ESI) positive ion 426 (M+H)⁺; negative ion 424 (M−H)⁻.

### EXAMPLE 47

### N-(4-{2,4-Diamino-6-[(benzyloxy)methyl]pyrimidin-5-yl}phenyl)propanamide

The title compound was synthesized according to the procedure described in Example 29, substituting propionic acid for 4-chlorobenzoic acid. ¹H NMR (300 MHz, DMSO-d₆) δ 1.10 (d, J=7.60 Hz, 3 H), 2.34 (q, J=7.57 Hz, 2 H), 3.96 (s, 2 H), 4.33 (s, 2 H), 5.64 (s, 2 H), 5.99 (s, 2 H), 7.12 (d, J=8.81 Hz, 2 H),7.15 (m, 2 H), 7.26 (m, 3 H), 7.63 (d, J=8.48 Hz, 2 H), and 9.91 (s, 1 H). MS (ESI) positive ion 378 (M+H)⁺; negative ion 376 (M−H)⁻.

### EXAMPLE 48

### 6-[(Benzyloxy)methyl]-5-{4-[(pyridin-4-ylmethyl)amino]phenyl}pyrimidine-2,4-diamine

The title compound was synthesized according to the procedure described in Example 2, substituting 4-pyridinecarboxaldehyde for 4-chlorobenzaldehyde. ¹H NMR (300 MHz, DMSO-d₆) δ 3.93 (s, 2 H), 4.32 (s, 2 H), 4.33 (d, J=7.12 Hz, 2 H), 5.49 (s, 2 H), 5.87 (s, 2 H), 6.45 (t, J=5.93 Hz, 1 H), 6.59 (d, J=8.48 Hz, 2 H), 6.90 (d, J=8.48 Hz, 2 H), 7.17 (m, 2 H), 7.26 (m, 3 H), 7.38 (d, J=5.76 Hz, 2 H), and 8.50 (m, 2 H). MS (ESI) positive ion 413 (M+H)⁺; negative ion 411 (M−H)⁻.

### EXAMPLE 49

### N-(4-Chlorobenzyl)-N-(4-{2,4-diamino-6-[(benzyloxy)methyl]pyrimidin-5-yl}phenyl)acetamide

To a solution of 4-chlorobenzyl(4-{2,4-diamino-6-[(benzyloxy)methyl]pyrimidin-5-yl}aniline from Example 2 (35 mg,0.08mmol) in CH₂Cl₂ (2 mL) at 0° C. was added acetyl chloride (0.09 mmol). The mixture was stirred at 0° C. for 10 minutes, at room temperature for 0.5 hour and concentrated under reduced pressure. The residue was purified by column chromatography to provide the title compound (35.1 mg, 90%). ¹H NMR (300 MHz, DMSO-d₆) δ 7.34-7.14 (m, 13H), 6.0 (s, 2H), 5.72 (s, 2H), 4.84 (s, 2H), 4.24 (s, 2H), 3.90 (s, 2H), 2H), 1.87 (s, 3H). MS (ESI) positive ion 488 (M+H)⁺; negative ion 486 (M−H)⁻.

### EXAMPLE 50

### 4-Chlorobenzyl(4-{2,4-diamino-6-[(benzyloxy)methyl]pyrimidin-5-yl}phenyl)formamide

A mixture of 4-chlorobenzyl(4-{2,4-diamino-6-[(benzyloxy)methyl]pyrimidin-5-yl}aniline from Example 2 (44.5 mg, 0.1 mmol), formic acid(140 mg, 3 mmol), and acetic anhydride(102 mg, 1 mmol) in 10 mL flask was heated at 60° C. for 1 hour. The mixture was concentrated under reduced pressure, the residue diluted with water, basified with 5% NaOH to a pH of 10 and extracted with CH₂Cl₂. The combined organic extracts were dried over MgSO4, filtered, concentrated under reduced pressure, and then purified by column chromatography to provide the title compound (42 mg, 88%). ¹H NMR (300 MHz, DMSO-d₆) δ 8.69 (s, 1H), 7.36-7.12 (m, 13H), 5.98 (s, 2H), 5.63 (s, 2H), 5.02 (s, 2H), 4.25 (s, 2H), 3.90 (s, 2H). MS (ESI) positive ion 474 (M+H)⁺; negative ion 472 (M−H)⁻.

### EXAMPLE 51

### 6-[(Benzyloxy)methyl]-5-{4-[(1H-imidazol-2-ylmethyl)amino]phenyl}pyrimidine-2,4-diamine

The title compound was synthesized according to the procedure described in Example 2, substituting 2-imidazolcarboxaldehyde for 4-chlorobenzaldehyde. ¹H NMR (300 MHz, DMSO-d₆) δ 3.96 (s, 2 H), 4.25 (d, J=5.76 Hz, 2 H), 4.34 (s, 2 H), 5.47 (br s, 2 H), 5.88 (s, 2 H), 6.12 (t, J=5.43 Hz, 1 H), 6.70 (d, J=8.48 Hz, 2 H), 6.84 (br s, 1 H), 6.92 (d, J=8.48 Hz, 2 H), 7.02 (br s, 1 H), 7.20 (m, 2 H), 7.28 (m, 3 H), and 11.87 (s, 1 H); MS (ESI) positive ion 402 (M+H)⁺; negative ion 400 (M−H)⁻.

### EXAMPLE 52

### 5-(4-{[2-(Benzyloxy)ethyl]amino}phenyl)-6-ethylpyrimidine-2,4-diamine

The titled compound was prepared according to the procedure described in Example 6, substituting 2-benzyloxyethanol for 4-cyanobenzyl alcohol used in Example 6. ¹H NMR (500 MHz, DMSO-d₆) δ ppm 0.96 (t, J=7.49 Hz, 3 H), 2.14 (q, J=7.59 Hz, 2 H), 3.26 (dd, J=11.54, 5.61 Hz, 2 H), 3.61 (t, J=5.77 Hz, 2 H), 4.53 (s, 2 H), 5.36 (s, 2 H), 5.67 (t, J=5.61 Hz, 1 H), 5.78 (s, 2 H), 6.66 (d, J=8.73 Hz, 2 H), 6.86 (d, J=8.42 Hz, 2 H), 7.34 (m, 5 H); MS (ESI) positive ion 364(M+H)⁺.

### EXAMPLE 53

### 6-[(Benzyloxy)methyl]-5-(4-{[(6-chloropyridin-3-yl)methyl]amino}phenyl)pyrimidine-2,4-diamine

The titled compound was prepared according to the procedure described in Example 2, substituting 6-chloro-pyridine-3-carbaldehyde (Oida, Sadao et al. Chem. Pharm. Bull.; EN; 48; 5; 2000; 694-707) for 4-chloro-benzaldehyde. ¹H NMR (300 MHz, DMSO-d₆) δ 8.45 (d, J=2.4 Hz, 1H), 7.85 (dd, J=8.5 Hz, J=2.4 Hz, 1H), 7.49 (d, J=8.5 Hz, 1H), 7.28-7.15 (m, SH), 6.92 (d, J=8.8 Hz, 2H), 6.63 (d, J=8.8 Hz, 2H), 6.39 (t, J=6.1 Hz, 1H), 5.87 (s, 2H), 5.50 (bs, 2H), 4.32, 4.33 (s, s, 4H), 3.93 (s, 2H). MS (ESI) positive ion 447 (M+H)⁺; negative ion 445 (M−H)⁻.

### EXAMPLE 54

### N-benzyl-3-(2,6-diamino-5-{4-[(4-chlorobenzyl)amino]phenyl}pyrimidin-4-yl)propanamide

### EXAMPLE 54A

### N-Benzyl-3-[2,6-diamino-5-(4-nitrophenyl)-pyrimid-4-yl]-propionamide

To a solution of 3-[2,6-diamino-5-(4-nitrophenyl)pyrimidine-4-yl]-propionic acid hydrochloride from Example 61 B(50 mg, 0.147 mmol) in DMF (1.5 mL) was added benzylamine (0.032 mL, 0.29 mmol) and TBTU (50 mg, 0.155 mmol). The mixture was stirred at room temperature for 16 hours, diluted with water and the resulting solid was filtered and rinsed with diethyl ether. The title compound was collected as a bright yellow solid (47 mg, 82%).

### EXAMPLE 54B

### N-Benzyl-3-[2,6-diamino-5-(4-nitrophenyl)-pyrimid-4-yl]-propionamide

A mixture of N-Benzyl-3-[2,6-diamino-5-(4-nitrophenyl)-pyrimid-4-yl]-propionamide from Example 54A (45 mg, 0.115 mmol) and 10% Pd/C (5 mg) in glacial acetic acid (1 mL) was stirred under an atmosphere of H₂ for 3 hours at room temperature. The mixture was filtered through Celite, rinsed with MeOH and concentrated under reduced pressure. The title compound was recovered as a white solid (40 mg, 96%).

### EXAMPLE 54C

### N-Benzyl-3-(2,6-diamino-5-{4-[(4-chlorobenzyl)amino]phenyl}pyrimidin-4-yl)propanamide

To a stirred solution of N-benzyl-3-[2,6-diamino-5-(4-nitrophenyl)-pyrimid-4-yl]-propionamide from Example 54B (40 mg, 0.11 mmol) in MeOH (1.11 mL) was added 4-chlorobenzaldehyde (15 mg, 0.11 mmol). After 30 minutes at room temperature, the reaction was cooled to 0° C., glacial acetic acid (0.03 mL, 0.5 mmol) was added followed by NaCNBH₃ (8 mg, 0.12 mmol). The mixture warmed to room temperature over 1 hour, diluted with saturated NaHCO₃ (5 mL) and extracted with EtOAc (2×5 mL). The combined organic layers were washed with brine (5 mL), dried over MgSO₄, filtered and concentrated under reduced pressure. Purification by reverse-phase HPLC (0-70% CH₃CN, aqueous NH₄OAc) provided an off white powder (10 mg, 18%). ¹H NMR (300 MHz, DMSO-d₆) δ 8.24 (t, J=5.93 Hz, 1H), 7.39-7.44 (m, 4H), 7.16-7.31 (m, 5 H), 6.86 (d, J=8.48 Hz, 2H), 6.61 (d, J=8.48 Hz, 2H), 6.34 (t, 6.10, 1H), 5.70 (s, 2H), 5.30 (s, 2H), 4.26 (d, J=6.10 Hz, 2H), 4.19 (d, J=5.76 Hz, 2H), 3.21-3.42 (m, 4H). MS (ESI) positive ion 487 (M+H)⁺; negative ion 485 (M−H)⁻.

### EXAMPLE 55

### 3-(2,6-Diamino-5-{4-[(4-chlorobenzyl)amino]phenyl}pyrimidin-4-yl)-N-phenylpropanamide

### EXAMPLE 55A

### 3-[2,6-Diamino-5-(4-nitro-phenyl)-pyrimidin-4-yl]-N-phenyl-propionamide

The titled compound was prepared according to the procedure described in Example 61A-C, substituting aniline for n-butylamine used in Example 61C.

### EXAMPLE 55B

### 3-(2,6-Diamino-5-{4-[(4-chlorobenzyl)amino]phenyl}pyrimidin-4-yl)-N-phenylpropanamide

3-(2,6-Diamino-5-({4-nitro-phenyl}pyrimidin-4-yl)-N-phenylpropanamide from Example 55A (55 mg, 0.145 mmol) was combined with 10% Pd/C (6 mg, ) in glacial acetic acid (1.4 mL) and placed under an atmosphere of H₂. The mixture was stirred for 3.5 hours at room temperature, filtered through Celite, rinsed with MeOH and concentrated under reduced pressure. The residue was dissolved in MeOH (1.4 mL) and 4-chlorobenzaldehyde (20 mg, 0.145 mmol) was added. The mixture was cooled to 0° C., glacial acetic acid (0.03 mL) and NaCNBH₃ were added. The mixture was warmed to room temperature, stirred for 1.5 hour, diluted with aqueous NaHCO₃ (5 mL) and extracted with EtOAc (2×10 mL). The combined organic layers were washed with brine, dried over MgSO₄, filtered and concentrated under reduced pressure. The residue was purified by reverse-phase HPLC (0-70% CH₃CN, aqueous NH₄OAc) to provide a white solid (10 mg, 15%). ¹H NMR (300 MHz, DMSO-d₆) δ 9.86 (s, 1H), 7.53 (d, J=7.80 Hz, 2H), 7.38-7.43 (m, 4H), 7.25 (t, J=7.97 Hz, 2H), 6.99 (t, J=7.29 Hz, 1H), 6.88 (d, J=8.48 Hz, 2H), 6.61 (d, J=8.48 Hz, 2H), 6.35 (t, J=5.76, 1H), 5.82 (s, 2H), 5.43 (s, 2H), 4.25 (d, J=5.76 Hz, 2H), 2.53-2.57 (m, 2H), 2.41-2.46 (m, 2H). MS (ESI) positive ion 473 (M+H)⁺; negative ion 471 (M−H)⁻.

### EXAMPLE 56

### 6-[(Benzyloxy)methyl]-5-{14-[(1-pyridin-4-ylethyl)amino]phenyl}pyrimidine-2,4-diamine

NaBH₃CN (10 mg, 0.15 mmol) was added to a mixture of 5-(4-amino-phenyl)-6-benzyloxymethyl-pyrimidine-2,4-diamine from Example 2 (32 mg, 0.1 mmol), 1-pyridin-4-yl-ethanone (12 μL, 0.11 mmol) and acetic acid (10 μL) in methanol (2 mL). The mixture was heated to 50° C. for 2 hours after which three more portions of reagents (acetic acid (10 μL×3), 1-pyridin-4-yl-ethanone (12 μL×3) and NaBH₃CN (10 mg×3)) were added with an interval of 1 hour. The mixture was then cooled to room temperature, partitioned between ethyl acetate and aqueous NaHCO₃ (30 mL, 1:1). The separated organic phase was washed with brine, dried (MgSO₄), filtered and concentrated under reduced pressure. The residue was purified on silica gel with ethyl acetate/methanol (10/1) to provide the title compound (10 mg). ¹H NMR (300 MHz, DMSO-d₆) δ 8.49 (d, J=6.1 Hz, 2H), 7.42 (d, J=6.1 Hz, 2H), 7.30-7.12 (m, SH), 6.85 (d, J=8.5 Hz, 2H), 6.53 (d, J=8.5 Hz, 2H), 6.35 (d, J=6.8 Hz, 1H), 5.87 (s, 2H), 5.45 (bs, 2H), 4.54-4.45 (m, 1H), 4.29 (s, 2H), 3.90 (s, 2H), 1.44 (d, J=7.1 Hz, 2H). MS (ESI) positive ion 427 (M+H)⁺; negative ion 425 (M−H)⁻.

### EXAMPLE 57

### 4-{1-[(4-{2,4-Diamino-6-[(benzyloxy)methyl]pyrimidin-5-yl}phenyl)amino]ethyl }benzonitrile

The title compound was synthesized according to the procedure described in Example 56, substituting 4-cyanoacetophenone for 4-acetylpyridine. ¹H NMR (300 MHz, DMSO-d₆) δ 1.43 (d, J=6.78 Hz, 3 H), 3.90 (s, 2 H), 4.28 (s, 2 H), 4.58 (t, J=6.60 Hz, 1 H), 5.45 (s, 2 H), 5.87 (s, 2 H), 6.39 (d, J=6.44 Hz, 1 H), 6.51 (d, J=8.82 Hz, 2 H), 6.84 (d, J=8.48 Hz, 2 H), 7.13 (dd, J=6.95, 2.88 Hz, 2 H), 7.25 (m, 3 H), 7.62 (d, J=8.48 Hz, 2 H), and 7.78 (d, J=8.14 Hz, 2 H). MS (ESI) positive ion 451 (M+H)⁺; 449 (M−H)⁻.

### EXAMPLE 58

### 6-[(Benzyloxy)methyl]-5-{4-[(4-methoxybenzyl)amino]phenyl}pyrimidine-2,4-diamine

The titled compound was prepared according to the procedure described in Example 2, substituting 4-methoxy-benzaldehyde for 4-chloro-benzaldehyde. ¹H NMR (300 MHz, DMSO-d₆) δ 7.32 (d, J=8.5 Hz, 2H), 7.28-7.15 (m, 5H), 6.90 (d, J=8.5 Hz, 2H), 6.89 (d, J=8.5 Hz, 2H), 6.62 (d, J=8.5 Hz, 2H), 6.22 (t, J=5.8 Hz, 1H), 5.87 (s, 2H), 5.47 (bs, 2H), 4.33 (s, 2H), 4.19 (d, J=5.8 Hz, 2H), 3.95 (s, 2H), 3.72 (s, 3H). MS (ESI) positive ion 442 (M+H)⁺; negative ion 440 (M−H)⁻.

### EXAMPLE 59

### 6-[(Benzyloxy)methyl]-5-(4-{[1-(4-chlorophenyl)ethyl]amino}phenyl)pyrimidine-2,4-diamine

The title compound was synthesized according to the procedure described in Example 56, substituting 4-chloroacetophenone for 4-acetylpyridine. ¹H NMR (500 MHz, DMSO-d₆) δ 1.41 (d, J=6.71 Hz, 3 H), 3.90 (s, 2 H), 4.29 (d, J=2.44 Hz, 2 H), 4.48 (pentet, J=6.60 Hz, 1 H), 5.46 (s, 2 H), 5.88 (s, 2 H), 6.30 (d, J=6.71 Hz, 1 H), 6.52 (d, J=8.54Hz, 2 H), 6.84 (d, J=8.54 Hz, 2H), 7.14 (dd, J=7.17, 2.29Hz, 2 H), 7.24 (m, 3 H), 7.36 (m, 2 H), and 7.44 (m, 2 H). MS (ESI) positive ion 460 (M+H)⁺; negative ion 458 (M−H)⁻.

### EXAMPLE 60

### 6-[(Benzyloxy)methyl]-5-{4-[(cyclohexylmethyl)amino]phenyl}pyrimidine-2,4-diamine

The titled compound was prepared according to the procedure described in Example 2, substituting cyclohexanecarbaldehyde for 4-chloro-benzaldehyde. ¹H NMR (300 MHz, DMSO-d₆) δ 7.32-7.18 (m, 5H), 6.90 (d, J=8.5 Hz, 2H), 6.59 (d, J=8.8 Hz, 2H), 5.86 (s, 2H), 5.70 (t, J=5.8 Hz, 1H), 5.49 (bs, 2H), 4.35 (s, 2H), 3.97 (s, 2H), 2.86 (t, J=5.8 Hz, 2H), 1.88-0.90(m, 11H). MS (ESI) positive ion 418 (M+H)⁺; negative ion 416 (M−H)⁻.

### EXAMPLE 61

### N-butyl-3-(2,6-diamino-5-{4-[(4-chlorobenzyl)amino]phenyl}pyrimidin-4-yl)propanamide

### EXAMPLE 61A

### 3-[2,6-Diamino-5-(4-nitro-phenyl)-pyrimidin-4-yl]-propionic acid methyl ester

To an ice-cooled solution of 3.24 g (20.0 mmol) of 4-nitrophenylacetonitrile and 130 mg (1.06 mmol) of 4-N,N-dimethylaminopyridine in 40 mL of CH₂Cl₂ was added 8.4 mL (60 mmol) of triethylamine, followed by 4.0 mL (32 mmol) of methyl 4-chloro-4-oxobutyrate dropwise over 1 minute. The mixture was stirred at 0° C. for 1 hour, and concentrated under reduced pressure. The residue was taken up in 80 mL of 0.5M HCl, and extracted with ethyl acetate (3×40 mL). The combined organic layers were back extracted with brine (1×40 mL), dried over MgSO₄, and filtered. The solution was cooled with an ice bath, 25 mL of methanol was added, followed by 25 mL of 2M trimethylsilyldiazomethane in diethyl ether. The solvents were removed under reduced pressure and the oily residue triturated with methanol to give a granular solid afterwhich the solvent was removed under reduced pressure. The solid was dissolved in 40 mL of tetrahydrofuran, afterwhich a premixed solution of 1.91 g (20 mmol) of guanidine hydrochloride and 20 mL of sodium methoxide in 25 mL of methanol, containing some solid KCl was added. The mixture was heated to reflux for 15 minutes, cooled and concentrated under reduced pressure. The residue was taken up in 50 mL of water, and filtered. The precipitate was washed with 10 mL of water, and then with 25 mL of methanol. The crude product was recrystallized from 40 mL of methanol to provide the title compound (700 mg, 11%) as a yellow powder.

### EXAMPLE 61B

### 3-[2,6-Diamino-5-(4-nitro-phenyl)-pyrimidin-4-yl]-propionic acid

To 694 mg (2.19 mmol) of 3-[2,6-diamino-5-(4-nitro-phenyl)-pyrimidin-4-yl]-propionic acid methyl ester from Example 61A was added 25 mL of 1M HCl. The suspension was heated to 90° C. for 1 hour during which time the starting ester dissolved. The mixture was concentrated under reduced pressure to provide the title compound (762 mg, 100%) of the product as a light brown solid containing a small amount of water.

### EXAMPLE 61C

### N-Butyl-3-{2,6-diamino-5-[4-(4-chloro-benzylamino)-phenyl]-pyrimidin-4-yl}-propionamide

To 37 mg (0.50 mmol) of n-butylamine was added a solution of 50 mg (0.15 mmol) of 3-[2,6-diamino-5-(4-nitro-phenyl)-pyrimidin-4-yl]-propionic acid and 50 mg (0.16 mmol) of O-benzotriazol-1-yl-N,N,N′,N′-tetramethyluronium tetrafluoroborate (TBTU) in 1 mL of DMF. The mixture was shaken at ambient temperature for 18 hours, diluted with 5 mL of water and 1 mL of saturated NaHCO₃. The precipitated amide was filtered, washed with water, dried on the filter. To the crude amide was added 5 mg of 10% Pd-C and 1 mL of acetic acid. The mixture was stirred under 1 atmosphere of H₂ for 4 hours, then filtered. The acetic acid was removed under reduced pressure. To the residue was added 11 mg of 4-chlorobenzaldehyde (0.079 mmol), 0.5 mL of methanol, and 0.5 mL of acetic acid. The solution was stirred for 10 minutes afterwhich 20 mg (0.32 mmol) of sodium cyanoborohydride was added. The mixture was stirred for 30 minutes at ambient temperature, then the mixture was concentrated under reduced pressure. The residue was taken up in aqueous NaHCO₃ (3 mL) and extracted with ethyl acetate (2×1 mL). The combined ethyl acetate layers were back extracted with brine (1×1 mL), dried over MgSO₄, filtered, and concentrated under reduced pressure to provide the crude benzylamine. The product was purified by reverse phase HPLC, eluting with a 5 to 100 CH₃CN/aq. 0.1% trifluoroacetic acid gradient to provide 15 mg (15%) of N-butyl-3-{2,6-diamino-5-[4-(4-chloro-benzylamino)-phenyl]-pyrimidin-4-yl}-propionamide TFA salt as a foam. ¹H NMR (300 MHz, d₆-DMSO) δ 11.94 (s, 1H); 8.07 (s, 1H), 7.87 (t, 1H, J=5.4 Hz), 7.39 (m, 4H), 6.91 (d, 2H, J=8.8 Hz), 6.65 (d, 2H, J=8.8 Hz), 6.61 (s, 2H), 4.28 (s, 2H), 3.00 (m, 2H), 2.44 (t, 2H, J=7.1 Hz), 2.27 (t, 2H, J=7.0 Hz), 1.33 (m, 2H), 1.22 (m, 2H), 0.85 (t, 3H, J=7.3 Hz); MS (ESI) m/z 453 [M+H]⁺.

### EXAMPLE 62

### 3-(2,6-Diamino-5-{4-[(4-chlorobenzyl)amino]phenyl}pyrimidin-4-yl)-N-(3-methylphenyl)propanamide

The titled was prepared according to the same procedure described for Example 61, substituting 16 mg (0.15 mmol) of m-toluidine for n-butylamine used in Example 61C. The yield was 10 mg (9%) of the TFA salt as a foam. ¹H NMR (300 MHz, d₆-DMSO) mixture of rotamers δ 11.87 (s, 1H), 9.86 (s, 1H), 8.10 (s, 1H), 7.35 (m, 9.5H), 7.29 (t, 1H, J=7.6 Hz), 6.90 (m, 3.5H), 6.64 (m, 4H), 4.26 (m, 2H), 2.50 (m, 4H), 2.26 (s, 3H), 2.26 (s, minor, 3H); MS (ESI) m/z 485 [M−H]⁺.

### EXAMPLE 63

### 6-[(Benzyloxy)methyl]-5-{4-[(4-chlorobenzyl)oxy]phenyl}pyrimidine-2,4-diamine

### EXAMPLE 63A

### 4-(2,4-Diamino-6-benzyloxymethyl-pyrimidin-5-yl)-phenol

To 654 mg (2.03 mmol) of 5-(4-amino-phenyl)-6-benzyloxymethyl-pyrimidine-2,4-diamine from Example 2 was added 7 mL of I M H₂SO₄. The solution was stirred at ambient temperature until all of the starting aniline had dissolved, then it was cooled with an ice bath. To the cold suspension was added a solution of 168 mg (2.43 mmol) of sodium nitrite dissolved in a minimum amount of water, and the reaction was stirred for 10 minutes at 0° C., warmed to ambient temperature over 10 minutes, then heated to reflux for 40 minutes. The reaction was cooled, treated with 10 mL of ethyl acetate and 15 mL of saturated NaHCO₃. A gummy precipitate formed which could be dissolved in a small amount of methanol, then partitioned between the aqueous and organic layers to speed dissolution. The aqueous layer was extracted with additional ethyl acetate (2×10 mL), then back extracted with brine (1×10 mL), dried over MgSO₄, filtered, and concentrated to a foam. The residue was taken up in methanol and reconcentrated to provide the title phenol (600 mg, 92%) as a yellow foam.

### EXAMPLE 63B

### 6-Benzyloxymethyl-5-[4-(4-chloro-benzyloxy)-phenyl]-pyrimidine-2,4-diamine

To a solution of 48 mg (0.15 mmol) of 4-(2,4-diamino-6-benzyloxymethyl-pyrimidin-5-yl)-phenol from Example 63A in 0.5 mL of ethanol was added 0.15 mmol of potassium ethoxide in 60 μL of ethanol. The solution was stirred for 2 minutes, then 31 mg (0.15 mmol) of 4-chlorobenzyl bromide was added. The reaction was stirred for 4.5 hour then 1 mL of water was added, and a yellow precipitate formed. The precipitate was collected, washed with water, then with diethyl ether, and dried on the filter to provide 44 mg (67%) of a pale yellow solid. Similar products prepared from other halides could be purified by recrystallization from i-PrOH/H₂O or ethanol/H₂O. Alternatively, the products could be purified by reverse phase HPLC, eluting with a 5 to 100% CH₃CN in 0.1% aq. TFA gradient to give the final compounds as its TFA salt. ¹H NMR (300 MHz, d₆-DMSO) δ 7.49 (m, 4H), 7.28 (m, 3H), 7.17 (m, 4H), 7.03 (d, 2H, J=8.8 Hz), 5.95 (s, 2H), 5.58 (s, 2H), 5.12 (s, 2H), 4.32 (s, 2H), 3.94 (s, 2H); MS (ESI) m/z 447 [M+H]⁺.

### EXAMPLE 64

### 6-[(Benzyloxy)methyl]-5-(4-{[(4-chlorobenzyl)amino]methyl}phenyl)pyrimidine-2,4-diamine

### EXAMPLE 64A

### 4-(2,4-Diamino-6-benzyloxymethyl-pyrimidin-5-yl)-benzonitrile

The titled compound was prepared according to the procedure described in Example 2, substituting 4-cyanophenylacetonitrile for 4-nitrophenylacetonitrile used in Example 2A. ¹HNMR (DMSO-d₆, 300MHz), δ 7.81 (d, J=8.5 Hz, 2H), 7.40 (d, J=8.5 Hz, 2H), 7.32-7.23 (m, 3H), 7.15-7.09 (m, 2H), 6.11 (s, 2H), 5.85 (s, 2H), 4.30 (s, 2H), 3.95 (s, 2H); MS (ESI) m/e 332 (M+H)⁺.

### EXAMPLE 64B

### 5-(4-Aminomethyl-phenyl)-6-benzyloxymethyl-pyrimidine-2,4-diamine

To a stirred suspension of phenylcyanide (600 mg, 1.8 mmol) from Example 67A in 1.0 N of NH₃/MeOH was added Raney Ni (75 mg, prewashed with MeOH and THF). The reaction flask was capped with a hydrogen balloon and hydrogenated at 60° C. for 4 hours. The almost clear solution was cooled to ambient temperature, filtered through celite, concentrated under reduced pressure to provide the titled compound as a beige solid (450 mg, 74% yield). ¹HNMR (DMSO-d₆, 300MHz), δ 27.36 (d, J=8.1 Hz, 2H), 7.34-7.12 (m, 5H), 7.15 (d, J=8.1 Hz, 2H), 5.97 (s, 2H), 5.55 (s, 2H), 4.44 (br m, 2H), 4.33 (s, 2H), 3.96 (s, 2H); MS (ESI) m/e 336 (M+H)⁺.

### EXAMPLE 64C

### 6-[(Benzyloxy)methyl]-5-(4-{[(4-chlorobenzyl)amino]methyl}phenyl)pyrimidine-2,4-diamine

The titled compound was prepared according to the procedure described in Example 2D, substituting benzylamine from Example 64B for the aniline used in Example 2D. ¹HNMR (DMSO-d₆, 300MHz), δ 7.45-7.12 (m, 13H), 5.97 (s, 2H), 5.57 (s, 2H), 5.24 (br m, 1H), 4.48 (s, 2H), 4.32 (s, 2H), 3.96 (s, 2H), 3.70 (s, 2H); MS (ESI) m/e 460, 462 (M+H)⁺.

### EXAMPLE 65

### 5-[4-(Benzylamino)phenyl]-6-[(benzyloxy)methyl]pyrimidine-2,4-diamine

The title compound was synthesized according to the procedure described in Example 2, substituting benzaldehyde for 4-chlorobenzaldehyde. ¹H NMR (400 MHz, DMSO-d₆) δ 3.95 (s, 2 H), 4.28 (d, J=5.83 Hz, 2 H), 4.33 (s, 2 H), 5.48 (s, 2 H), 5.85 (s, 2 H), 6.29 (t, J=5.98 Hz, 1 H), 6.63 (d, J=8.59 Hz, 2 H), 6.89 (d, J=8.59 Hz, 2 H), 7.18 (dd, J=7.83, 1.69 Hz, 2 H), 7.26 (m, 4 H), 7.34 (m, 2 H), and 7.40 (m, 2 H). MS (ESI) positive ion 412 (M+H)⁺; negative ion 410 (M−H)⁻.

### EXAMPLE 66

### 6-[(Benzyloxy)methyl]-5-(4-{[(4-nitrophenyl)amino]methyl}phenyl)pyrimidine-2,4-diamine

A mixture of benzylamine (30 mg, 0.089 mmol) from Example 64B, diisopropylethylamine in excess (150 μL), 1-fluoro-4-nitrobenzene (19 μL, 0.18 mmol) in 1.0 mL of NMP was heated at 200° C. for 20 minutes in a Personal Chemistry Optimizer MicroWave reactor. Solvent was removed on a Savant SpeedVac, and the crude residue was purified on a preparative TLC to provide the title compound as a light yellow solid (10 mg, 24% yield). ¹HNMR (DMSO-d₆, 300MHz), δ 7.98 (d, J=9.5 Hz, 2H), 7.86 (t, J=6.1 Hz, 1H), 7.37 (d, J=8.1 Hz, 2H), 7.30-7.09 (m, 9H), 6.7 (d, J=9.5 Hz, 2H), 6.01 (s, 2H), 5.58 (s, 2H), 4.46 (d, J=6.1 Hz, 2H), 4.29 (s, 2H), 3.94 (s, 2H); MS (ESI) m/e 460, 462 (M+H)⁺.

### EXAMPLE 67

### N-(4-{2,4-Diamino-6-[(benzyloxy)methyl]pyrimidin-5-yl}benzyl)-N′-propylurea

To a stirred suspension of amine (25 mg, 0.075 mmol) in 1.0 mL of methylene chloride was added Et₃N in excess (100 μL), and propyl isocyanate (11 mL, 0.11 mmol). The resulting mixture was refluxed for 1 hour, cooled to room temperature, concentrated under reduced pressure, and the crude residue purified on a Gilson Preparative HPLC to provide the title compound as an off-white solid (15 mg, 48% yield). ¹HNMR (DMSO-d₆, 300MHz), δ 7.33-7.13 (m, 9H), 6.29 (t, J=5.9 Hz, 1H), 5.98 (s, 2H), 5.95 (t, J=5.4 Hz, 1H), 5.57 (s, 2H), 4.33 (s, 2H), 4.26 (dd, J=6.1 Hz, 2H), 3.95 (s, 2H), 2.99 (q, J=6.1 Hz, 2H), 1.40 (q, J=6.8 Hz, 2H), 0.84 (t, J=7.5 Hz, 3H); MS (ESI) m/e 421 (M+H)⁺.

### EXAMPLE 68

### 4-{[(4-{2,4-Diamino-6-[(cyclobutylmethoxy)methyl]pyrimidin-5-yl}phenyl)amino]methyl}benzonitrile

### EXAMPLE 68A

### Cyclobutylmethoxyacetic acid

To NaH (1.72 g of 60%) in THF (10 ml) was added a solution of cyclobutanemethanol (3.0 g, 35 mmol) in THF (10 ml) at −15° C. The mixture was stirred at 25° C. for 1 hour, concentrated under reduced pressure afterwhich sodium chloroacetate (5.2 g, 45mmol) in DMSO (100 ml) was added. The mixture was stirred at room temperature for 20 hours, then diluted with 300 ml water and extracted with hexane (100 ml×2). The aqueous phase was acidified with 2N HCl to pH 2, and then extracted with ethyl acetate (100 ml×2). The combined ethyl acetate layers were washed twice with H₂O (100 ml) and dried over MgSO₄, filtered and concentrated under reduced pressure to provide the title cyclobutylmethoxyacetic acid as a pale yellow oil (4.2 g, 83%), which was used in the next step without purification.

### EXAMPLE 68B

### 1-Cyano-1-(4-nitropheny)-3-cyclobutylmethoxyacetone

To the cyclobutylmethoxyacetic acid from Example 68A (1.44 g, 10 mmol) in CH₂Cl₂ (20 ml) was added slowly oxalylchloride (2.54 g, 20 mmol) and DMF (0.1 ml) at 0° C. The mixture was stirred at 0° C. for 0.5 hour then at room temperature for 1 hour before being concentrated under reduced pressure. The residue in fresh CH₂Cl₂ (10 ml) was added to a solution of nitrophenyl acetonitrile (1.62, 10 mmol) in CH₂Cl₂ with Et₃N (1.68 ml, 12 mmol) and DMAP (162 mg). The mixture was stirred overnight, then H₂O (30 ml) and HCl (2N, 1 ml) were added. The organic layer was washed with brine (2×10 ml), dried over MgSO4, filtered, concentrated under reduced pressures and purification by crystallization using ethyl acetate and hexane to provided the title 1-cyano-1-(4-nitropheny)-3-cyclobutylmethoxy acetone (1.87 g, 65%). ¹H NMR (300 MHz, DMSO-D₆) δ 8.23 (d, J=9.0 Hz, 2H), 7.96 (d, J=9.0 Hz, 2H), 4.39 (s, 2H), 3.48 (d, J=9.0 Hz, 2H), 2.57 (m, 1H), 2.51 (s, 1H), 2.04-1.71 (m, 6H). MS (ESI) positive ion 287 (M+H)⁺.

### EXAMPLE 68C

### 2,4-Diamino-6-[(cyclobutylmethoxy)methyl]-5-(4-nitrophenyl)pyrimidine

To 1-cyano-1-(4-nitropheny)-3-cyclobutylmethoxyacetone from Example 68B (450 mg, 1.56 mmol) was added CH₂N₂ (3 mmol in 10 ml ether) at 0° C. The mixture was stirred for 10 minutes then the solvent removed under reduced pressure to provide a vinyl ether compound (470 mg, 99%). ¹H NMR (300 MHz, DMSO-D₆) δ 8.26 (d, J=9.0 Hz, 2H), 7.89 (d, J=9.0 Hz, 2H), 4.63 (s, 2H), 4.08 (s, 3H), 2.54 (d, J=9.0 Hz, 1H), 2.59 (m, 1H), 2.04-1.71 (m, 6H). MS (ESI) positive ion 301 (M+H)^{+,} negative ion 299 (M−H)⁻. To the residue (453 mg, 1.5 mmol) in ethanol (20 mL) was added guanidine carbonate (270 mg, 1.5 mmol). The mixture was refluxed for 2 hours, then cooled to room temperature and filtered. The solid was washed with ethyl acetate (10 ml×3) and dried to provide the title compound as pale yellow crystals (385 mg, 78%).¹H NMR (300 MHz, DMSO-D₆) δ 8.24(d, J=9.0 Hz, 2H), 7.51 (d, J=9.0 Hz, 2H), 6.14 (s, 2H), 3.91 (s, 2H), 3.17 (d, J=9.0 Hz, 1H), 2.31 (m, 1H), 1.90-1.49 (m, 6H). MS (ESI) positive ion 330 (M+H)⁺, negative ion 328 (M−H)⁻.

### EXAMPLE 68D

### 2,4-Diamino-6-[(cyclobutylmethoxy)methyl]-5-(4-aminophenyl)pyrimidine

To 2,4-diamino-6-[(cyclobutylmethoxy)methyl]-5-(4-nitrophenyl)pyrimidine from Example 68C (380 mg, 1.15 mmol) in 10 ml methanol was added Pd/C (122 mg, 10%). The mixture was stirred under 1 atmosphere of hydrogen at room temperature for 2 hours, then filtered and concentrated under reduced pressure to provide the title compound (330 mg, 96%). ¹H NMR (300 MHz, DMSO-d₆) δ 6.85 (d, J=9.0 Hz, 2H), 6.60 (d, J=9.0 Hz, 2H), 5.85 (s, 2H), 5.20 (s, 2H), 5.11 (s, 2H), 3.85 (s, 2H), 3.21 (d, J=9.0 Hz, 1H), 2.39 (m, 1H), 1.95-1.59 (m, 6H). MS (ESI) positive ion 300 (M+H)⁺, negative ion 298(M−H)⁻.

### EXAMPLE 68E

### 4-{[(4-{2,4-Diamino-6-[(cyclobutylmethoxy)methyl]pyrimidin-5-yl}phenyl)amino]methyl}benzonitrile

To 2,4-diamino-6-[(cyclobutylmethoxy)methyl]-5-(4-aminophenyl)pyrimidine from Example 68D (30 mg, 0.1 mmol) in methanol (2 mL) and a buffer solution of acetic acid and sodium acetate (1 mL, pH 4-5) was added 4-cyanobenzaldehyde (14.5 mg, 0.11 mmol) followed by NaBH₃CN (76 mg, 0.12 mmol). The mixture was stirred at room temperature for 2 hours afterwhich the solvents were removed under reduced pressure. The residue was purified by column chromatography to provide the title compound (23 mg, 55%). ¹H NMR (300 MHz, DMSO-d₆) δ 7.80 (d, J=9.0 Hz, 2H), 7.58 (d, J=9.0 Hz, 2H), 6.90 (d, J=9.0 Hz, 2H), 6.59 (d, J=9.0 Hz, 2H), 6.49 (t, J=3.0 Hz, 1H), 5.98 (s, 2H), 5.62(s, 2H), 4.40 (d, J=6.0 Hz, 2H), 3.84 (s, 2H), 3.18 (d, J=6.0 Hz, 2H), 2.35 (m, 1H), 1.91-1.54 (m, 6H). MS (ESI) positive ion 415 (M+H)⁺; negative ion 413 (M−H)⁻.

### EXAMPLE 69

### 4-[(4-{2,4-Diamino-6-[(benzyloxy)methyl]pyrimidin-5-yl}phenoxy)methyl]benzonitrile

The titled compound was prepared by the same procedure described in Example 63, substituting 4-cyanobenzyl bromide for 4-chlorobenzyl bromide used in Example 63B. The product was purified by recrystallization from i-PrOH/H₂O or ethanol/H₂O to give 40 mg (58%) of a yellow solid. ¹H NMR (300 MHz, d₆-DMSO) δ 7.92 (d, 2H, J=8.1 Hz), 7.65 (d, 2H, J=8.1 Hz), 7.26 (m, 3H), 7.15 (m, 4H), 7.05 (m, 2H), 5.94 (s, 2H), 5.56 (s, 2H), 5.25 (s, 2H), 4.28 (s, 2H), 3.92 (s, 2H); MS (ESI) m/z 438 [M+H]⁺.

### EXAMPLE 70

### 5-{4-[(4-Chlorobenzyl)amino]phenyl}-6-[(tetrahydro-2H-pyran-2-ylmethoxy)methyl]pyrimidine-2,4-diamine

### EXAMPLE 70A

### 5-(4-Amino-phenyl)-6-(tetrahydro-pyran-2-ylmethoxymethyl)-pyrimidine-2,4-diamine

The titled compound was prepared according to the procedure described in Example 68A-D, substituting 2-tetrahydropyranmethanol for cyclobutanemethanol used in Example 68A.

### EXAMPLE 70B

### 5-{4-[(4-Chlorobenzyl)amino]phenyl}-6-[(tetrahydro-2H-pyran-2-ylmethoxy)methyl]pyrimidine-2,4-diamine

To a stirred solution of 5-(4-amino-phenyl)-6-(tetrahydropyran-2-ylmethoxymethyl)-pyrimidine-2,4-diamine from Example 70A (60 mg, 0.182 mmol) in MeOH (1.8 mL) was added 4-chlorobenzaldehyde (26 mg, 0.182 mmol). After 30 minutes at room temperature, the reaction was cooled to 0° C. Glacial acetic acid (0.04 mL, 0.6 mmol) was added followed by NaCNBH₃ (14 mg, 0.218 mmol). The reaction warmed to room temperature over 1 h. To the reaction was added saturated NaHCO₃ (7 mL). It was washed with EtOAc (3×7 mL). The combined organic layers were washed with brine (7 mL), dried over MgSO₄, and concentrated. The residue was triturated with isopropanol and filtered to give an off-white solid (29 mg, 35%). ¹H NMR (300 MHz, DMSO-d₆) δ 7.36-7.43 (m, 4H), 6.88 (d, J=8.48 Hz, 2H), 6.59 (d, J=8.48 Hz, 2H), 6.36 (t, J=5.93 Hz, 1H), 5.85 (s, 2H), 5.47 (s, 2H), 4.27 (d, J=5.67 Hz, 2H), 3.86 (s, 2H), 3.76 (dd, J=11.02, 2.88 Hz, 1H), 3.17-3.25 (m, 3H), 3.10-3.13 (m, 1H), 1.69-1.71 (m, 1H), 1.32-1.42 (m, 4H), 1.03-1.07 (m, 1H). MS (ESI) positive ion 454 (M+H)⁺; negative ion 452 (M−H)⁻.

### EXAMPLE 71

### 6-[(Benzyloxy)methyl]-5-[4-({[6-(trifluoromethyl)pyridin-3-yl]methyl}amino)phenyl]pyrimidine-2,4-diamine

The titled compound was prepared according to the procedure described in Example 2, substituting 6-trifluolo-pyridine-3-carbaldehyde for 4-chloro-benzaldehyde. ¹H NMR (300 MHz, DMSO-d₆) δ 8.79-7.76 (m, 1H), 8.01-7.98 (m, 2H), 7.30-7.15 (m, 5H), 6.92 (d, J=8.5 Hz, 2H), 6.63 (d, J=8.5 Hz, 2H), 6.49 (t, J=6.1 Hz, 1H), 5.87 (s, 2H), 5.48 (bs, 2H), 4.45 (d, J=6.1 Hz, 2H), 4.31 (s, 2H), 3.93 (s, 2H). MS (ESI) positive ion 438 (M+H)⁺; negative ion 436 (M−H)⁻.

### EXAMPLE 72

### 4-[(4-{2,4-Diamino-6-[(benzyloxy)methyl]pyrimidin-5-yl}benzyl)amino]benzonitrile

The titled compound was prepared according to the procedure described in Example 66, substituting 4-fluorobenzonitrile for 1-fluoro-4-nitrobenzene used in Example 66. ¹HNMR (DMSO-d₆, 300MHz), δ 7.92 (m, 1H), 7.49-7.09 (m, 9H), 7.43 (d, J=8.8 Hz, 2H), 6.68 (d, J=8.8 Hz, 2H), 6.17 (s, 2H), 5.89 (s, 2H), 4.38 (d, J=6.1 Hz, 2H), 4.31 (s, 2H), 3.96 (s, 2H); MS (ESI) m/e 437 (M+H)⁺.

### EXAMPLE 73

### 3-[(4-{2,4-Diamino-6-[(benzyloxy)methyl]pyrimidin-5-yl}phenoxy)methyl]benzonitrile

The titled compound was prepared by the same procedure described for Example 63, substituting 3-cyanobenzyl bromide for 4-chlorobenzyl bromide used in Example 63B. The product was purified by recrystallization from i-PrOH/H₂O or ethanol/H₂O to give 33 mg (49%) of a yellow solid. ¹H NMR (300 MHz, d₆-DMSO) δ 7.95 (s, 1H), 7.82 (m, 2H), 7.61 (t, 1H, J=7.8 Hz), 7.26 (m, 3H), 7.16 (m, 4H), 7.06 (m, 2H), 5.94 (s, 2H), 5.57 (s, 2H), 5.18 (s, 2H), 4.29 (s, 2H), 3.96 (s, 2H); MS (ESI) m/z 438 [M+H]⁺.

### EXAMPLE 74

### 5-{[(4-{2,4-Diamino-6-[(benzyloxy)methyl]pyrimidin-5-yl}phenyl)amino]methyl}pyridine-2-carbonitrile

The titled compound was prepared according to the procedure described in Example 2, substituting 6-cyano-pyridine-3-carbaldehyde (Ashimori, Atsuyuki et al.; Chem.Pharm.Bull.; EN; 38; 9; 1990; 2446-2458) for 4-chloro-benzaldehyde. ¹H NMR (300 MHz, DMSO-d₆) δ 8.45 (d, J=2.4 Hz, 1H), 7.85 (dd, J=8.5 Hz, J=2.4 Hz, 1H), 7.49 (d, J=8.5 Hz, 1H), 7.28-7.15 (m, 5H), 6.92 (d, J=8.8 Hz, 2H), 6.63 (d, J=8.8 Hz, 2H), 6.39 (t, J=6.1 Hz, 1H), 5.87 (s, 2H), 5.50 (bs, 2H), 4.32, 4.33 (s, s, 4H), 3.93 (s, 2H). MS (ESI) positive ion 447 (M+H)⁺; negative ion 445 (M−H)⁻.

### EXAMPLE 75

### 6-[(Benzyloxy)methyl]-5-{4-[2-(4-chlorophenyl)ethoxy]phenyl}pyrimidine-2,4-diamine

The titled compound was prepared by the same procedure described for Example 63, substituting 4-chlorophenethyl bromide (Saunders, W. H. Jr.; Williams, R. A. J. Am. Chem. Soc. 1957, 79, 3712) for 4-chlorobenzyl bromide used in Example 63B. The product was purified by reverse phase HPLC to give 17 mg (19%) of a yellow solid. ¹H NMR (300 MHz, d₆-DMSO) δ 11.69 (s, 1H), 8.24 (s, 1H), 7.62 (bs, 2H), 7.38 (s, 4H), 7.27 (m, 5H), 7.12 (m, 2H), 7.03 (m, 2H), 6.90 (m, 1H), 4.47 (s, 2H), 4.22 (t, 2H, J=6.4 Hz), 4.15 (s, 2H), 3.06 (t, 2H, J=6.4 Hz); MS (ESI) m/z 461 [M+H]⁺.

### EXAMPLE 76

### 6-[(Benzyloxy)methyl]-5-[4-(pyridin-3-ylmethoxy)phenyl]pyrimidine-2,4-diamine

The titled compound was prepared by the same procedure described for Example 63, substituting 3-(chloromethyl)pyridine hydrochloride for 4-chlorobenzyl bromide used in Example 63B and adding an additional 0.15 mmol potassium ethoxide. Heating at reflux was required to complete the substitution reaction The product was purified by HPLC to give 21 mg (22%) of the bis[trifluoroacetate] salt as a foam. ¹H NMR (300 MHz, d₆-DMSO) δ 11.65 (s, 1H), 8.77 (d, 1H, J=1.4 Hz), 8.61 (dd, 1H, J=4.7, 1.7 Hz), 8.27 (s, 1H), 8.00 (ddd, 1H, J=7.8, 2.0, 1.7 Hz), 7.59 (bs, 2H), 7.54 (ddd, J=7.8, 5.1, 0.7 Hz), 7.32 (m, 6H), 7.15 (m, 4H), 6.95 (s, 1H), 5.22 (s, 2H), 4.52 (s, 2H), 4.17 (s, 2H); MS (ESI) m/z 414 [M+H]⁺.

### EXAMPLE 77

### 6-[(Benzyloxy)methyl]-5-{4-[(tetrahydro-2H-pyran-4-ylmethyl)amino]phenyl}pyrimidine-2,4-diamine

### EXAMPLE 77A

### 4-(Hydroxymethyl)tetrahydropyran

To an ice-cooled solution of 2.60 g (20.0 mmol) of tetrahydropyran-4-carboxylic acid in 8 mL of THF was added 21 mL (21 mmol) of 1.0M borane in THF. The reaction was stirred at 0 C for 1 h, then quenched by dropwise addition of 2 mL of water. After stirring for 10 min at ambient temperature, solid K₂CO₃ was added and swirled until free flowing. The salts were filtered, and the supernatant was concentrated to 1.25 g (54%) of 4-(hydroxymethyl)tetrahydropyran as a colorless oil.

### EXAMPLE 77B

### 4-Tetrahydropyran carboxaldehyde

To a solution of 116 mg (1.00 mmol) of 4-(hydroxymethyl)tetrahydropyran from Example 77A in 2 mL of CH₂Cl₂ was added 424 mg (1.0 mmol) of the Dess-Martin periodinane. The mixture was stirred at ambient temperature for 1 h, then filtered through diatomaceous earth. The filter cake was washed with about 3 mL of CH₂Cl₂, then the tilted aldehyde solution was used directly in the next step.

### EXAMPLE 77C

### 6-[(Benzyloxy)methyl]-5-{4-[(tetrahydro-2H-pyran-4-ylmethyl)amino]phenyl}pyrimidine-2,4-diamine

To a solution of 1 mmol of 4-tetrahydropyran carboxaldehyde in about 5 mL of CH₂Cl₂ was added 200 mg of 5-(4-amino-phenyl)-6-benzyloxymethyl-pyrimidine-2,4-diamine. The solution was stirred for 10 min, then concentrated in vacuo. The residue was dissolved in 2 mL of methanol and 0.4 mL of glacial acetic acid. To the solution was added 100 mg (1.59 mmol) of sodium cyanoborohydride. The reaction was stirred at ambient temperature for 1 h, then concentrated in vauco. The residue was dissolved in 5 mL of 2M NaOH_{(aq.)}, and extracted with ethyl acetate (2×5 mL). The combined organic layers were back extracted with 2M NaOH (1×5 mL), and brine (1×5 mL), dried over MgSO₄, filtered, and concentrated to 222 mg of a foam. A 99 mg portion of this crude product was purified by reverse phase HPLC, eluting with 5% to 100% CH₃CN in aq 0.1% trifluoroacetic acid to give 48 mg (27%) of the product as its bis(trifluoroacetate) salt. ¹H NMR (300 MHz, d₆-DMSO) δ 11.51 (s, 1H), 8.27 (s, 1H), 7.53 (bs, 2H), 7.32 (m, 6H), 6.90 (d, 2H, J=8.5 Hz), 6.85 (s, 1H), 6.62 (d, 2H, J=8.8 Hz), 4.48 (s, 2H), 4.20 (s, 2H), 3.95 (dd, 2H, J=11.5, 2.7 Hz), 3.28 (td, 2H, J=11.7, 2.0 Hz), d.92 (d, 2H, J=6.4 Hz), 1.79 (m, 1H), 1.69 (m, 2H), 1.27 (dd, 1H, J=11.9, 4.4 Hz), 1.19 (dd, 1H, J=11.9, 3.7 Hz); MS (ESI) m/z 420 [M+H]⁺.

### EXAMPLE 78

### 6-[(Benzyloxy)methyl]-5-(4-{[4-(trifluoromethoxy)benzyl]amino}phenyl)pyrimidine-2,4-diamine

The titled compound was prepared according to the procedure described in Example 2, substituting 4-trifluoromethoxy-benzaldehyde for 4-chloro-benzaldehyde. ¹H NMR (300 MHz, DMSO-d₆) δ 7.52 (d, J=8.5 Hz, 2H), 7.34-7.15 (m, 7H), 6.91 (d, J=8.5 Hz, 2H), 6.62 (d, J=8.5 Hz, 2H), 6.38 (t, J=5.8 Hz, 1H), 5.87 (s, 2H), 5.47 (bs, 2H), 4.34-4.30 (m, 4H), 3.94 (s, 2H); MS (ESI) positive ion 496 (M+H)⁺; negative ion 494 (M−H)⁻.

### EXAMPLE 79

### 5-{4-[(4-Chlorobenzyl)amino]phenyl}-6-[(cyclohexylmethoxy)methyl]pyrimidine-2,4-diamine

### EXAMPLE 79A

### 5-(4-Amino-phenyl)-6-cyclohexylmethoxymethyl-pyrimidine-2,4-diamine

The titled compound was prepared according to the procedure described in Example 68A-D, substituting cyclohexanemethanol for cyclobutanemethanol used in Example 68A.

### EXAMPLE 79B

### 5-{4-[(4-Chlorobenzyl)amino]phenyl}-6-[(cyclohexylmethoxy)methyl]pyrimidine-2,4-diamine

5-(4-Amino-phenyl)-6-cyclohexylmethoxymethyl-pyrimidine-2,4-diamine from Example 79A (50 mg, 0.14 mmol) was combined with 10% Pd/C (5 mg), taken up in glacial acetic acid (1.4 mL) and placed under an atmosphere of H₂. The reaction was complete after 4 h at room temperature. It was filtered through Celite, rinsed with MeOH and concentrated. The residue was dissolved in MeOH (1.4 mL) and 4-chlorobenzaldehyde (20 mg, 0.14 mmol) was added. The reaction is cooled to 0° C. and glacial acetic acid (0.03 mL) and NaCNBH₃ (11 mg, 0.17 mmol) were added. The reaction warmed to room temperature and was complete after 1.0 h. Saturated NaHCO₃ (5 mL) and the aqueous is extracted with EtOAc (2×10 mL). The combined organic layers were washed with brine, dried over MgSO4 and concentrated. The resulting residue was purified by reverse-phase HPLC (0-70% CH₃CN, aqueous NH₄OAc) to give a white powder (6 mg, 10%). ¹H NMR (300 MHz, DMSO-d₆) δ 7.36-7.42 (m, 4H), 6.88 (d, J=8.48 Hz, 2H), 6.58 (d, J=8.48 Hz, 2H), 6.36 (t, J=5.93 Hz, 1H), 5.86 (s, 2H), 5.50 (s, 2H), 4.27 (d, J=5.76 Hz, 2H), 3.80 (s, 2H), 3.11-3.24 (m, 1H), 2.99 (d, J=6.44 Hz, 2H), 1.54-1.58 (m, 4H), 1.23-1.35 (m, 2H), 1.08-1.14 (m, 2H), 0.76-0.90 (m, 2H). MS (ESI) positive ion 452 (M+H)⁺; negative ion 450 (M−H)⁻.

### EXAMPLE 80

### 4-{[(4-{2,4-Diamino-6-[(benzyloxy)methyl]pyrimidin-5-yl}phenyl)amino]methyl}pyridine-2-carbonitrile

The titled compound was prepared according to the procedure described in Example 2, substituting 2-cyano-pyridine-4-carbaldehyde (Ashimori, Atsuyuki et al.; Chem. Pharm. Bull.; EN; 38; 9; 1990; 2446-2458) for 4-chloro-benzaldehyde. ¹H NMR (300 MHz, DMSO-d₆) δ 8.68 (d, J=5.1 Hz, 1H), 8.0 (s, 1H), 7.72 (d, J=5.1 Hz, 1H), 7.30-7.15 (m, 5H), 6.92 (d, J=8.8 Hz, 2H), 6.60 (d, J=8.8 Hz, 2H), 6.52 (t, J=6.1 Hz, 1H), 5.88 (s, 2H), 5.52 (bs, 2H), 4.42 (d, J=6.1 Hz, 2H), 4.31 (s, 2H), 3.93 (s, 2H). MS (ESI) positive ion 438 (M+H)⁺; negative ion 436 (M−H)⁻.

### EXAMPLE 81

### 6-[(4-{2,4-Diamino-6-[(benzyloxy)methyl]pyrimidin-5-yl}benzyl)amino]nicotinonitrile

The titled compound was prepared according to the procedure described in Example 66, substituting 6-chloronictonitrile for 1-fluoro-4-nitrobenzene used in Example 66. ¹HNMR (DMSO-d₆, 300MHz), δ 8.42-8.36 (m, 1H), 8.17-8.11 (m, 1H), 7.37-7.12 (m, 8H), 6.64 (d, J=8.8 Hz, 2H), 5.97 (s, 2H), 5.57 (s, 2H), 4.60 (d, J=6.1 Hz, 2H), 4.31 (s, 2H), 3.94 (s, 2H); MS (ESI) m/e 438 (M+H)⁺.

### EXAMPLE 82

### 5-{4-[(4-Chlorobenzyl)amino]phenyl}-6-{[(3-chlorobenzyl)oxy]methyl}pyrimidine-2,4-diamine

To a solution of {2,6-diamino-5-[4-(4-chloro-benzylamino)-phenyl]-pyrimidin-4-yl}-methanol (51.2 mg, 0.14 mmol) in anhydrous DMF (0.5 mL) was added 3-chlorobenzylbromide (16.5 mL, 0.126 mmol). The reaction was stirred a few minutes and then sodium t-butoxide was added (14.6 mg, 0.154 mmol) and stirred for 24 h. Dilute reaction mixture to 2 mL with methanol and purify by preperative HPLC (5-100% CH₃CN/0.1% TFA in H₂O, Synergi Hydro-RP by Phenomenex). The desired fractions were concentrated in vaccuo to yield 22.3 mg (22%) of white solid. ¹H NMR (300 MHz, DMSO-d₆) δ 11.53 (s, 1H), 8.25 (br s, 1H), 7.41 (s, 4H), 7.34-7.37 (m, 3H), 7.25-7.20 (m, 1H), 6.91 (s, 1H), 6.88 (s, 1H), 6.65 (s, 1H), 6.62 (s, 1H), 4.47 (s, 2H), 4.28 (d, 2H), 4.18 (s, 2H). MS (DCI/NH₃): 480, 482, 484 (M+H)⁺.

### EXAMPLE 83

### 5-{4-[(4-Chlorobenzyl)amino]phenyl}-6-{[(2-methylbenzyl)oxy]methyl}pyrimidine-2,4-diamine

The titled compound was prepared according to the procedure described in Example 82, substituting 2-methylbenzylbromide (16.5 mL, 0.126 mmol) for 3-chlorobenzylbromide (16.5 mL, 0.126 mmol) to yield 29.0 mg (29%) of the titled compound as a white solid. ¹H NMR (300 MHz, DMSO-d₆) δ 11.51 (s, 1H), 8.25 (br s, 1H), 7.41 (s, 4H), 7.09-7.20 (m, 4H), 6.93 (s, 1H), 6.90 (s, 1H), 6.65 (s, 1H), 6.62 (s, 1H), 4.44 (s, 2H), 4.29 (d, 2H), 4.17 (s, 2H), 2.22 (s, 3H). MS (DCI/NH₃) m/e 460,462 (M+H)⁺.

### EXAMPLE 84

### 6-[(Benzyloxy)methyl]-5-{4-[(4-nitrobenzyl)amino]phenyl}pyrimidine-2,4-diamine

The title compound was synthesized according to the procedure described in Example 2, substituting 4-nitrobenzaldehyde for 4-chlorobenzaldehyde. ¹H NMR (300 MHz, DMSO-d₆) δ 3.95 (s, 2 H), 4.32 (s, 2 H), 4.44 (d, J=6.10 Hz, 2 H), 5.61 (s, 2 H), 5.97 (s, 2 H), 6.55 (t, J=6.27 Hz, 1 H), 6.60 (d, J=8.48 Hz, 2 H), 6.90 (d, J=8.48 Hz, 2 H), 7.16 (dd, J=7.46, 2.03 Hz, 2 H), 7.25 (m, 3 H), 7.66 (d, J=8.81 Hz, 2 H), and 8.20 (ddd, J=9.07, 2.54, 2.29 Hz, 2 H); MS (ESI) positive ion 457 (M+H)⁺; negative ion 455 (M−H)⁻.

### EXAMPLE 85

### 6-Ethyl-5-{4-[(4-nitrobenzyl)amino]phenyl}pyrimidine-2,4-diamine

The titled compound was prepared according to the procedure described in Example 1, substituting 4-nitro-benzaldehyde for 4-chloro-benzaldehyde. ¹H NMR (300 MHz, DMSO-d₆) δ 8.22 (d, J=8.8 Hz, 2H), 7.67 (d, J=8.8 Hz, 2H), 6.86.(d, J=8.5 Hz, 2H), 6.62 (d, J=8.5 Hz, 2H), 6.51 (t, J=6.1 Hz, 1H), 5.71 (s, 2H), 5.23 (bs, 2H), 4.44 (d, J=6.1 Hz, 2H), 2.10 (q, J=7.5 Hz, 2H), 0.94 (t, J=7.5 Hz, 3H). MS (ESI) positive ion 365 (M+H)⁺.

### EXAMPLE 86

### 6-[(Benzyloxy)methyl]-5-(4-{[(2-chloropyridin-4-yl)methyl]amino}phenyl)pyrimidine-2,4-diamine

The titled compound was prepared according to the procedure described in Example 2, substituting 2-chloro-pyridine-4-carbaldehyde (Watson, Samuel E. et al.; Heterocycles; EN; 48; 10; 1998; 2149-2156) for 4-chloro-benzaldehyde. ¹H NMR (300 MHz, DMSO-d₆) δ 8.34 (d, J=5.1 Hz, 1H), 7.49 (s, 1H), 7.42 (d, J=5.1 Hz, 1H), 7.30-7.15 (m, 5H), 6.91 (d, J=8.5 Hz, 2H), 6.60 (d, J=8.5 Hz, 2H), 6.49 (t, J=6.5 Hz, 1H), 5.87 (s, 2H), 5.50 (bs, 2H), 4.36 (d, J=6.5 Hz, 2H), 4.31 (s, 2H), 3.93 (s, 2H). MS (ESI) positive ion 447 (M+H)⁺; negative ion 445 (M−H)⁻.

### EXAMPLE 87

### 6-[(Benzyloxy)methyl]-5-{4-[(pyrimidin-5-ylmethyl)amino]phenyl}pyrimidine-2,4-diamine

### EXAMPLE 87A

### Pyrimidine-5-carboxaldehyde

A modified procedure of Rho and Abuh (Syn. Commun. 1994, 24, 253-256) was followed for the preparation of the titled aldehyde. Under nitrogen, to a solution of 5-bromopyrimidine (1 g, 6.3 mmol) in 60 mL anhydrous THF, was added BuLi (2.5 M, 2.6 mL, 6.5 mmol) at −78° C. The resulting yellow solution was stirred for 20 min, after which ethyl formate (0.55 mL, 6.7 mmol) was added dropwise over 5 min. After 20 min, the reaction was quenched with 1.5 M THF/HCl solution (4.5 mL, 6.7 mmol). The cold bath was removed, and the reaction mixture was stirred for 1 h. THF was removed in vacco, 10 mL of water was then added. The mixture was extracted with CHCl₃ (2×10 mL), and the combined organics were dried (MgSO₄) and concentrated. The crude product was purified via flash column chromatography (5% MeOH/CHCl₃) to give 0.35 g (51%) of the titled pyrimidine-5-carboxaldehyde.

### EXAMPLE 87B

### 6-[(Benzyloxy)methyl]-5-{4-[(pyrimidin-5-ylmethyl)amino]phenyl}pyrimidine-2,4-diamine

The titled compound was then prepared according to the procedure described in Example 2, substituting pyrimidine-5-carboxaldehyde from Example 87A for 4-chlorobenzaldehyde used in Example 2. ¹H NMR (300 Hz, DMSO-d₆) δ 9.08 (s, 1H), 8.82 (s, 2H), 7.27-7.16 (m, 5H), 6.93 (d, J=9 Hz, 2H), 6.66 (d, J=6 Hz, 2H), 4.32 (s, 2H), 3.94 (s, J=6 Hz, 1H), 5.87 (s, 2H), 5.50 (s, 2H), 4.35 (d, J=6 Hz, 2H), 4.32 (s, 2H), 3.94 (s, 2H). MS(ESI) positive ion 414 (M+H)⁺; negative ion 412 (M−H)⁻.

### EXAMPLE 88

### 6-[(Benzyloxy)methyl]-5-{4-[(thien-2-ylmethyl)amino]phenyl}pyrimidine-2,4-diamine

The titled compound was then prepared according to the procedure described in Example 2, substituting thiophene-2-carboxaldehyde for 4-chlorobenzaldehyde used in Example 2. ¹H NMR (300 Hz, DMSO-d₆) δ 7.38 (d, J=6 Hz, 1H), 7.31-7.17 (m, 5H), 7.20 (d, J=3 Hz, 1H), 6.98 (d, J=3 Hz, 1H), 6.92 (d, J=9 Hz, 2H), 6.68 (d, J=9 Hz, 2H), 6.33 (t, J=6 Hz, 1H), 5.86 (s, 2H), 5.48 (s, 2H), 4.46 (d, J=6 Hz, 2H), 4.33 (s, 2H), 3.95 (s, 2H). MS(ESI) positive ion 418 (M+H)⁺; negative ion 416 (M−H)⁻.

### EXAMPLE 89

### 6-[(Benzyloxy)methyl]-5-{4-[(thien-3-ylmethyl)amino]phenyl}pyrimidine-2,4-diamine

The titled compound was then prepared according to the procedure described in Example 2, substituting thiophene-3-carboxaldehyde for 4-chlorobenzaldehyde used in Example 2. ¹H NMR (300 Hz, DMSO-D₆) δ 7.49 (d, J=6 Hz, 1H), 7.38 (d, J=3 Hz, 1H), 7.31-7.18 (m, 5H), 7.13 (d, J=6 Hz, 1H), 6.91 (d, J=9 Hz, 2H), 6.65 (d, J=6 Hz, 2H), 6.17 (t, J=6 Hz, 1H), 5.86 (s, 2H), 5.49 (s, 2H), 4.33 (s, 2H), 4.26 (d, J=6 Hz, 2H), 3.96 (s, 2H). MS(ESI) positive ion 418 (M+H)⁺; negative ion 416 (M−H)⁻.

### EXAMPLE 90

### 6-[(Benzyloxy)methyl]-5-[4-({[1-(4-chlorophenyl)ethyl]amino}methyl)phenyl]pyrimidine-2,4-diamine

To 4-{(2,4-diamino-6-[(cyclobutylmethoxy)methyl]pyrimidin-5-yl}benzylamine from Example 64 (33.5 mg, 0.1 mmol)) in methanol (2 ml) and a buffer solution of acetic acid and sodium acetate (1 ml, pH 4-5) was added 4-chloroacetophenone (18.5 mg, 0.12 mmol), then NaBH₃CN (76 mg, 0.12 mmol). The reaction mixture was stirred at r.t for 2 h before the solvents were removed on evaporator under pressure. The residue was purified by column chromatography to yield the titled compound (29 mg, 61%). ¹H NMR (300 MHz, DMSO-d₆) δ 7.38 (s, 5H), 7.32-7.13 (m, 8H), 5.97 (s, 2H), 5.56(s, 2H), 4.32 (s, 2H), 3.96 (s, 2H), 3.73 (q, J=6.0 Hz, 1H), 3.52 (d, J=3.0 Hz, 2H), 1.26(d, 3H). MS (ESI) positive ion 474 (M+H)⁺; negative ion 472 (M−H)⁻.

### EXAMPLE 91

### 6-[(Benzyloxy)methyl]-5-(4-{[2-(4-nitrophenyl)ethyl]amino}phenyl)pyrimidine-2,4-diamine

The titled compound was prepared according to the procedure described in Example 2, substituting (4-nitro-phenyl)-acetaldehyde (Ashwell, Mark A. et al, Bioorg.Med.Chem.Lett.; EN; 11; 24; 2001; 3123-3128) for 4-chloro-benzaldehyde. ¹H NMR (300 MHz, DMSO-d₆) δ 8.18 (d, J=8.8 Hz, 2H), 7.60 (d, J=8.8 Hz, 2H), 7.32-7.18 (m, 5H), 6.93 (d, J=8.5 Hz, 2H), 6.64 (d, J=8.5 Hz, 2H), 5.87 (s, 2H), 5.81 (t, J=6.1 Hz, 1H), 5.47 (bs, 2H), 4.35 (s, 2H), 3.97 (s, 2H), 3.38-3.22 (m, 2H), 3.01 (t, J=7.1 Hz, 2H). MS (ESI) positive ion 471 (M+H)⁺.

### EXAMPLE 92

### 6-[(Benzyloxy)methyl]-5-(4-{[2-(4-chlorophenyl)ethyl]amino}phenyl)pyrimidine-2,4-diamine

The titled compound was prepared according to the procedure described in Example 2, substituting (4-chloro-phenyl)-acetaldehyde for 4-chloro-benzaldehyde. ¹H NMR (300 MHz, DMSO-d₆) δ 7.38-7.18 (m, 5H), 6.93 (d, J=8.5 Hz, 2H), 6.63 (d, J=8.5 Hz, 2H), 5.87 (s, 2H), 5.76 (t, J=6.1 Hz, 1H), 5.51 (bs, 2H), 4.35 (s, 2H), 3.97 (s, 2H), 3.38-3.22 (m, 2H), 2.85 (t, J=7.1 Hz, 2H). MS (ESI) positive ion 460 (M+H)⁺; negative ion 458 (M−H)⁻.

### EXAMPLE 93

### 6-[(Benzyloxy)methyl]-5-{4-[(cycloheptylamino)methyl]phenyl}pyrimidine-2,4-diamine

The titled compound was prepared according to the procedure described in Example 90, substituting cycloheptanone for 4-chloroacetophenone used in Example 90 (78% yield). ¹H NMR (300 MHz, DMSO-D₆) δ 7.37-7.14 (m, 9H), 5.96 (s, 2H), 5.56(s, 2H), 4.31 (s, 2H), 3.96 (s, 2H), 3.73 (s, 2H), 2.64 (m, 1H), 1.88-1.33(m, 13H). MS (ESI) positive ion 432 (M+H)⁺; negative ion 430 (M−H)⁻.

### EXAMPLE 94

### 6-Benzyloxymethyl-5-[4-(pyridin-4-ylmethoxy)-phenyl]-pyrimidine-2,4-diamine

The titled compound was prepared by the same procedure described for Example 63, substituting 4-(chloromethyl)pyridine hydrochloride for 4-chlorobenzyl bromide used in Example 63B, and adding an additional 0.15 mmol potassium ethoxide. Heating at reflux was required to complete the substitution reaction. The product was purified by recrystallization from i-PrOH/H₂O or ethanol/H₂O to give 8 mg (13%) of a solid. ¹H NMR (300 MHz, d₆-DMSO) δ 8.59 (d, 2H, J=6.1 Hz), 7.48 (d, 2H, J=5.8 Hz), 7.27 (m, 3H), 7.15 (m, 4H), 7.04 (d, 2H, J=8.6 Hz), 5.94 (s, 2H), 5.59 (s, 2H), 5.15 (s, 2H), 4.26 (s, 2H), 3.88 (s, 2H); MS (ESI) m/z 414 [M+H]⁺.

1. A compound according to formula (I), 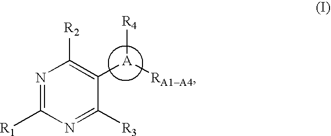
or a therapeutically suitable salt or prodrug thereof, wherein
R₁ is a member selected from the group consisting of hydrogen, alkyl, alkoxy, aryl, arylalkyl, cyano, cycloalkyl, cycloalkylalkyl, haloalkoxy, haloalkyl, halogen, heteroaryl, heteroarylalkyl, heterocycle, heterocyclealkyl, hydroxy, mercapto, nitro, and —NR_{A}R_{B};
R_{A} and R_{B} are each independently a member selected from the group consisting of hydrogen, alkoxycarbonyl, alkyl, alkylcarbonyl, alkoxysulfonyl, alkylsulfonyl, aryl, arylalkyl, and formyl;
R₂ is a member selected from the group consisting of hydrogen, alkyl, alkoxy, alkoxycarbonyl, aryl, arylalkyl, cyano, cycloalkyl, cycloalkylalkyl, haloalkoxy, haloalkyl, halogen, heteroaryl, heteroarylalkyl, heterocycle, heterocyclealkyl, hydroxy, mercapto, nitro, —NR_{C}R_{D}, and (NR_{C}R_{D})alkyl;
R_{C} and R_{D} are each independently a member selected from the group consisting of hydrogen, alkoxycarbonyl, alkenyl, alkyl, alkylcarbonyl, alkoxysulfonyl, alkylsulfonyl, aryl, arylalkyl, cycloalkyl, cycloalkylalkyl, formyl, and hydroxyalkyl;
R₃ is a member selected from the group consisting of alkenyl, alkenylalkoxyalkyl, alkenyloxy, alkenyloxyalkyl, alkoxy, alkoxyalkoxy, alkoxyalkoxyalkyl, alkoxyalkyl, alkoxycarbony, alkoxycarbonylalkyl, alkoxysulfonyl, alkylcarbonyl, alkylcarbonylalkyl, alkylcarbonyloxy, alkylsulfinyl, alkylsulfinylalkyl, alkylsulfonyl, alkylsulfonylalkyl, alkylthio, alkylthioalkyl, alkynyl, alkynylalkoxyalkyl, alkynyloxy, alkynyloxyalkyl, aryl, arylalkoxy, arylalkoxyalkyl, arylalkyl, arylalkylthio, arylalkylthioalkyl, aryloxy, aryloxyalkyl, arylthio, arylthioalkyl, carboxy, carboxyalkyl, cyanoalkyl, cycloalkenyl, cycloalkenylalkoxy, cycloalkenylalkoxyalkyl, cycloalkenylalkyl, cycloalkenylalkylthio, cycloalkenylalkylthioalkyl, cycloalkenyloxy, cycloalkenyloxyalkyl, cycloalkenylthio, cycloalkenylthioalkyl, cycloalkyl, cycloalkylalkoxy, cycloalkylalkoxyalkyl, cycloalkylalkyl, cycloalkylalkylthio, cycloalkylalkylthioalkyl, cycloalkyloxy, cycloalkyloxyalkyl, cycloalkylthio, cycloalkylthioalkyl, formyl, haloalkoxy, halogen, heteroaryl, heteroarylalkoxy, heteroarylalkoxyalkyl, heteroarylalkyl, heteroarylalkylthio, heteroarylalkylthioalkyl, heteroaryloxy, heteroaryloxyalkyl, heteroarylthio, heteroarylthioalkyl, heterocycle, heterocyclealkoxy, heterocyclealkoxyalkyl, heterocyclealkyl, heterocyclealkylthio, heterocyclealkylthioalkyl, heterocycleoxy, heterocycleoxyalkyl, heterocyclethio, heterocyclethioalkyl, hydroxy, hydroxyalkyl, mercapto, nitro, —NR_{E}R_{H}, (NR_{E}R_{H})alkyl, (NR_{E}R_{F})carbonylalkenyl (NR_{E}R_{F})carbonylalkyl, (NR_{E}R_{F})sulfonyl, and (NR_{E}R_{F})sulfonylalkyl;
R_{E} and R_{F} are each independently a member selected from the group consisting of hydrogen, alkoxyalkyl, alkoxyalkylcarbonyl, alkoxycarbonyl, alkoxysulfonyl, alkyl, alkylcarbonyl, alkylsulfonyl, alkylthioalkyl, alkylthioalkylcarbonyl, alkylthiocarbonyl, aryl, arylalkoxyalkyl, arylalkyl, arylcarbonyl, cycloalkyl, cycloalkylalkyl, cycloalkylcarbonyl, formyl, heteroaryl, heteroarylalkyl, heteroarylcarbonyl, heterocycle, heterocyclealkyl, heterocyclecarbonyl, (NZ₁Z₂)alkyl, and (NZ₁Z₂)carbonyl;
Z₁ and Z₂ are each independently a member selected from the group consisting of hydrogen, alkoxycarbonyl, alkyl, alkylcarbonyl, alkoxysulfonyl, alkylsulfonyl, aryl, arylalkyl, arylcarbonyl, cycloalkyl, cycloalkylalkyl, cycloalkylcarbonyl, formyl, heteroaryl, heteroarylalkyl, heteroarylcarbonyl, heterocycle, heterocyclealkyl, and heterocyclecarbonyl;
R₄ is a member selected from the group consisting of alkenyl, alkenyloxy, alkenyloxyalkyl, alkoxy, alkoxyalkoxy, alkoxyalkoxyalkyl, alkoxyalkyl, alkoxycarbonyl, alkoxycarbonylalkyl, alkoxysulfonyl, alkyl, alkylcarbonyl, alkylcarbonylalkyl, alkylcarbonyloxy, alkylsulfinyl, alkylsulfinylalkyl, alkylsulfonyl, alkylsulfonylalkyl, alkylthio, alkylthioalkyl, alkynyl, alkynyloxy, alkynyloxyalkyl, aryl, arylalkoxy, arylalkoxyalkyl, arylalkyl, arylalkylthio, arylalkylthioalkyl, aryloxy, aryloxyalkyl, arylthio, arylthioalkyl, carboxy, carboxyalkyl, cyano, cyanoalkyl, cycloalkenyl, cycloalkenylalkoxy, cycloalkenylalkoxyalkyl, cycloalkenylalkyl, cycloalkenylalkylthio, cycloalkenylalkylthioalkyl, cycloalkenyloxy, cycloalkenyloxyalkyl, cycloalkenylthio, cycloalkenylthioalkyl, cycloalkyl, cycloalkylalkoxy, cycloalkylalkoxyalkyl, cycloalkylalkyl, cycloalkylalkylthio, cycloalkylalkylthioalkyl, cycloalkyloxy, cycloalkyloxyalkyl, cycloalkylthio, cycloalkylthioalkyl, formyl, formylalkyl, haloalkoxy, haloalkyl, halogen, heteroaryl, heteroarylalkoxy, heteroarylalkoxyalkyl, heteroarylalkyl, heteroarylalkylthio, heteroarylalkylthioalkyl, heteroaryloxy, heteroaryloxyalkyl, heteroarylthio, heteroarylthioalkyl, heterocycle, heterocyclealkoxy, heterocyclealkoxyalkyl, heterocyclealkyl, heterocyclealkylthio, heterocyclealkylthioalkyl, heterocycleoxy, heterocycleoxyalkyl, heterocyclethio, heterocyclethioalkyl, hydroxy, hydroxyalkyl, mercapto, nitro, —NR_{G}R_{H}, (NR_{G}R_{H})alkyl, (NR_{G}R_{H})carbonyl, and (NR_{C}R_{H})sulfonyl;
R_{G} and R_{H} are each independently a member selected from the group consisting of hydrogen, alkoxyalkyl, alkoxyalkylcarbonyl, alkoxycarbonyl, alkoxysulfonyl, alkyl, alkylcarbonyl, alkylsulfonyl, alkylthioalkyl, alkylthioalkylcarbonyl, alkylthiocarbonyl, aryl, arylalkoxyalkyl, arylalkyl, arylcarbonyl, cycloalkyl, cycloalkylalkoxyalkyl, cycloalkylalkyl, cycloalkylcarbonyl, formyl, heteroaryl, heteroarylalkoxyalkyl, heteroarylalkyl, heteroarylcarbonyl, heterocycle, heterocyclealkoxyalkyl, heterocyclealkyl, heterocyclecarbonyl, (NZ₃Z₄)alkyl, and (NZ₃Z₄)carbonyl;
Z₃ and Z₄ are each independently a member selected from the group consisting of hydrogen, alkoxycarbonyl, alkoxysulfonyl, alkyl, alkylcarbonyl, alkylsulfonyl, aryl, arylalkyl, arylcarbonyl, cycloalkyl, cycloalkylalkyl, cycloalkylcarbonyl,.formyl, heteroaryl, heteroarylalkyl, heteroarylcarbonyl, heterocycle, heterocyclealkyl, and heterocyclecarbonyl;
A is a member selected from the group consisting of aryl, cycloalkyl, cycloalkenyl, heteroaryl, and heterocycle;
R_{A1}, R_{A2}, R_{A3}, and R_{A4} are each independently a member selected from the group consisting of hydrogen, alkenyl, alkenyloxy, alkoxy, alkoxyalkoxy, alkoxyalkoxyalkyl, alkoxyalkyl, alkoxycarbonyl, alkoxycarbonylalkyl, alkoxysulfonyl, alkyl, alkylcarbonyl, alkylcarbonylalkyl, alkylcarbonyloxy, alkylsulfinyl, alkylsulfinylalkyl, alkylsulfonyl, alkylsulfonylalkyl, alkylthio, alkylthioalkyl, alkynyl, aryl, carboxy, carboxyalkyl, cyano, cyanoalkyl, cycloalkyl, formyl, haloalkoxy, haloalkyl, halogen, heteroaryl, heterocyle, hydroxy, hydroxyalkyl, mercapto, nitro, —NR_{J}R_{K}, (NR_{J}R_{K})alkyl, (NR_{J}R_{K})carbonyl, and (NR_{J}R_{K})sulfonyl; and
R_{J} and R_{K} are each independently a member selected from the group consisting of hydrogen, alkoxycarbonyl, alkyl, alkylcarbonyl, alkoxysulfonyl, alkylsulfonyl, aryl, arylalkyl, and formyl.

2. A compound according to formula (Ia), 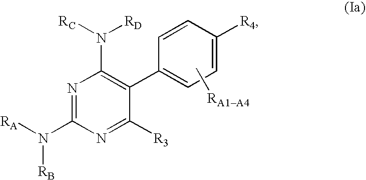
or a therapeutically suitable salt or prodrug thereof, wherein
R_{A} and R_{B} are each independently a member selected from the group consisting of hydrogen, alkoxycarbonyl, alkyl, alkylcarbonyl, alkoxysulfonyl, alkylsulfonyl, aryl, arylalkyl, and formyl;
R_{C} and R_{D} are each independently a member selected from the group consisting of hydrogen, alkoxycarbonyl, alkenyl, alkyl, alkylcarbonyl, alkoxysulfonyl, alkylsulfonyl, aryl, arylalkyl, cycloalkyl, cycloalkylalkyl, formyl, and hydroxyalkyl;
R₃ is a member selected from the group consisting of alkenyl, alkenylalkoxyalkyl, alkenyloxy, alkenyloxyalkyl, alkoxy, alkoxyalkoxy, alkoxyalkoxyalkyl, alkoxyalkyl, alkoxycarbony, alkoxycarbonylalkyl, alkoxysulfonyl, alkylcarbonyl, alkylcarbonylalkyl, alkylcarbonyloxy, alkylsulfinyl, alkylsulfinylalkyl, alkylsulfonyl, alkylsulfonylalkyl, alkylthio, alkylthioalkyl, alkynyl, alkynylalkoxyalkyl, alkynyloxy, alkynyloxyalkyl, aryl, arylalkoxy, arylalkoxyalkyl, arylalkyl, arylalkylthio, arylalkylthioalkyl, aryloxy, aryloxyalkyl, arylthio, arylthioalkyl, carboxy, carboxyalkyl, cyanoalkyl, cycloalkenyl, cycloalkenylalkoxy, cycloalkenylalkoxyalkyl, cycloalkenylalkyl, cycloalkenylalkylthio, cycloalkenylalkylthioalkyl, cycloalkenyloxy, cycloalkenyloxyalkyl, cycloalkenylthio, cycloalkenylthioaikyl, cycloalkyl, cycloalkylalkoxy, cycloalkylalkoxyalkyl, cycloalkylalkyl, cycloalkylalkylthio, cycloalkylalkylthioalkyl, cycloalkyloxy, cycloalkyloxyalkyl, cycloalkylthio, cycloalkylthioalkyl, formyl, haloalkoxy, halogen, heteroaryl, heteroarylalkoxy, heteroarylalkoxyalkyl, heteroarylalkyl, heteroarylalkylthio, heteroarylalkylthioalkyl, heteroaryloxy, heteroaryloxyalkyl, heteroarylthio, heteroarylthioalkyl, heterocycle, heterocyclealkoxy, heterocyclealkoxyalkyl, heterocyclealkyl, heterocyclealkylthio, heterocyclealkylthioalkyl, heterocycleoxy, heterocycleoxyalkyl, heterocyclethio, heterocyclethioalkyl, hydroxy, hydroxyalkyl, mercapto, nitro, —NR_{E}R_{H}, (NR_{E}R_{H})alkyl, (NR_{E}R_{F})carbonylalkenyl (NR_{E}R_{F})carbonylalkyl, (NR_{E}R_{F})sulfonyl, and (NR_{E}R_{F})sulfonylalkyl;
R_{E} and R_{F} are each independently a member selected from the group consisting of hydrogen, alkoxyalkyl, alkoxyalkylcarbonyl, alkoxycarbonyl, alkoxysulfonyl, alkyl, alkylcarbonyl, alkylsulfonyl, alkylthioalkyl, alkylthioalkylcarbonyl, alkylthiocarbonyl, aryl, arylalkoxyalkyl, arylalkyl, arylcarbonyl, cycloalkyl, cycloalkylalkyl, cycloalkylcarbonyl, formyl, heteroaryl, heteroarylalkyl, heteroarylcarbonyl, heterocycle, heterocyclealkyl, heterocyclecarbonyl, (NZ₁Z₂)alkyl, and (NZ₁Z₂)carbonyl;
Z₁ and Z₂ are each independently a member selected from the group consisting of hydrogen, alkoxycarbonyl, alkyl, alkylcarbonyl, alkoxysulfonyl, alkylsulfonyl, aryl, arylalkyl, arylcarbonyl, cycloalkyl, cycloalkylalkyl, cycloalkylcarbonyl, formyl, heteroaryl, heteroarylalkyl, heteroarylcarbonyl, heterocycle, heterocyclealkyl, and heterocyclecarbonyl;
R₄ is a member selected from the group consisting of alkenyl, alkenyloxy, alkenyloxyalkyl, alkoxy, alkoxyalkoxy, alkoxyalkoxyalkyl, alkoxyalkyl, alkoxycarbonyl, alkoxycarbonylalkyl, alkoxysulfonyl, alkyl, alkylcarbonyl, alkylcarbonylalkyl, alkylcarbonyloxy, alkylsulfinyl, alkylsulfinylalkyl, alkylsulfonyl, alkylsulfonylalkyl, alkylthio, alkylthioalkyl, alkynyl, alkynyloxy, alkynyloxyalkyl, aryl, arylalkoxy, arylalkoxyalkyl, arylalkyl, arylalkylthio, arylalkylthioalkyl, aryloxy, aryloxyalkyl, arylthio, arylthioalkyl, carboxy, carboxyalkyl, cyano, cyanoalkyl, cycloalkenyl, cycloalkenylalkoxy, cycloalkenylalkoxyalkyl, cycloalkenylalkyl, cycloalkenylalkylthio, cycloalkenylalkylthioalkyl, cycloalkenyloxy, cycloalkenyloxyalkyl, cycloalkenylthio, cycloalkenylthioalkyl, cycloalkyl, cycloalkylalkoxy, cycloalkylalkoxyalkyl, cycloalkylalkyl, cycloalkylalkylthio, cycloalkylalkylthioalkyl, cycloalkyloxy, cycloalkyloxyalkyl, cycloalkylthio, cycloalkylthioalkyl, formyl, formylalkyl, haloalkoxy, haloalkyl, halogen, heteroaryl, heteroarylalkoxy, heteroarylalkoxyalkyl, heteroarylalkyl, heteroarylalkylthio, heteroarylalkylthioalkyl, heteroaryloxy, heteroaryloxyalkyl, heteroarylthio, heteroarylthioalkyl, heterocycle, heterocyclealkoxy, heterocyclealkoxyalkyl, heterocyclealkyl, heterocyclealkylthio, heterocyclealkylthioalkyl, heterocycleoxy, heterocycleoxyalkyl, heterocyclethio, heterocyclethioalkyl, hydroxy, hydroxyalkyl, mercapto, nitro, —NR_{G}R_{H}, (NR_{G}R_{H})alkyl, (NR_{G}R_{H})carbonyl, and (NR_{G}R_{H})sulfonyl;
R_{G} and R_{H} are each independently a member selected from the group consisting of hydrogen, alkoxyalkyl, alkoxyalkylcarbonyl, alkoxycarbonyl, alkoxysulfonyl, alkyl, alkylcarbonyl, alkylsulfonyl, alkylthioalkyl, alkylthioalkylcarbonyl, alkylthiocarbonyl, aryl, arylalkoxyalkyl, arylalkyl, arylcarbonyl, cycloalkyl, cycloalkylalkoxyalkyl, cycloalkylalkyl, cycloalkylcarbonyl, formyl, heteroaryl, heteroarylalkoxyalkyl, heteroarylalkyl, heteroarylcarbonyl, heterocycle, heterocyclealkoxyalkyl, heterocyclealkyl, heterocyclecarbonyl, (NZ₃Z₄)alkyl, and (NZ₃Z₄)carbonyl;
Z₃ and Z₄ are each independently a member selected from the group consisting of hydrogen, alkoxycarbonyl, alkoxysulfonyl, alkyl, alkylcarbonyl, alkylsulfonyl, aryl, arylalkyl, arylcarbonyl, cycloalkyl, cycloalkylalkyl, cycloalkylcarbonyl, formyl, heteroaryl, heteroarylalkyl, heteroarylcarbonyl, heterocycle, heterocyclealkyl, and heterocyclecarbonyl;
R_{A1}, R_{A2}, R_{A3}, and R_{A4} are each independently a member selected from the group consisting of hydrogen, alkenyl, alkenyloxy, alkoxy, alkoxyalkoxy, alkoxyalkoxyalkyl, alkoxyalkyl, alkoxycarbonyl, alkoxycarbonylalkyl, alkoxysulfonyl, alkyl, alkylcarbonyl, alkylcarbonylalkyl, alkylcarbonyloxy, alkylsulfinyl, alkylsulfinylalkyl, alkylsulfonyl, alkylsulfonylalkyl, alkylthio, alkylthioalkyl, alkynyl, aryl, carboxy, carboxyalkyl, cyano, cyanoalkyl, cycloalkyl, formyl, haloalkoxy, haloalkyl, halogen, heteroaryl, heterocyle, hydroxy, hydroxyalkyl, mercapto, nitro, —NR_{J}R_{K}, (NR_{J}R_{K})alkyl, (NR_{J}R_{K})carbonyl, and (NR_{J}R_{K})sulfonyl; and
R_{J} and R_{K} are each independently a member selected from the group consisting of hydrogen, alkoxycarbonyl, alkyl, alkylcarbonyl, alkoxysulfonyl, alkylsulfonyl, aryl, arylalkyl, and formyl.

3. A compound according to formula (Ib), 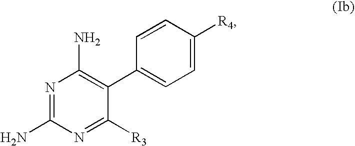
or a therapeutically suitable salt or prodrug thereof, wherein
R₃ is a member selected from the group consisting of alkenyl, alkenylalkoxyalkyl, alkenyloxy, alkenyloxyalkyl, alkoxy, alkoxyalkoxy, alkoxyalkoxyalkyl, alkoxyalkyl, alkoxycarbony, alkoxycarbonylalkyl, alkoxysulfonyl, alkylcarbonyl, alkylcarbonylalkyl, alkylcarbonyloxy, alkylsulfinyl, alkylsulfinylalkyl, alkylsulfonyl, alkylsulfonylalkyl, alkylthio, alkylthioalkyl, alkynyl, alkynylalkoxyalkyl, alkynyloxy, alkynyloxyalkyl, aryl, arylalkoxy, arylalkoxyalkyl, arylalkyl, arylalkylthio, arylalkylthioalkyl, aryloxy, aryloxyalkyl, arylthio, arylthioalkyl, carboxy, carboxyalkyl, cyanoalkyl, cycloalkenyl, cycloalkenylalkoxy, cycloalkenylalkoxyalkyl, cycloalkenylalkyl, cycloalkenylalkylthio, cycloalkenylalkylthioalkyl, cycloalkenyloxy, cycloalkenyloxyalkyl, cycloalkenylthio, cycloalkenylthioalkyl, cycloalkyl, cycloalkylalkoxy, cycloalkylalkoxyalkyl, cycloalkylalkyl, cycloalkylalkylthio, cycloalkylalkylthioalkyl, cycloalkyloxy, cycloalkyloxyalkyl, cycloalkylthio, cycloalkylthioalkyl, formyl, haloalkoxy, halogen, heteroaryl, heteroarylalkoxy, heteroarylalkoxyalkyl, heteroarylalkyl, heteroarylalkylthio, heteroarylalkylthioalkyl, heteroaryloxy, heteroaryloxyalkyl, heteroarylthio, heteroarylthioalkyl, heterocycle, heterocyclealkoxy, heterocyclealkoxyalkyl, heterocyclealkyl, heterocyclealkylthio, heterocyclealkylthioalkyl, heterocycleoxy, heterocycleoxyalkyl, heterocyclethio, heterocyclethioalkyl, hydroxy, hydroxyalkyl, mercapto, nitro, —NR_{E}R_{H}, (NR_{E}R_{H})alkyl, (NR_{E}R_{F})carbonylalkenyl (NR_{E}R_{F})carbonylalkyl, (NR_{E}R_{F})sulfonyl, and (NR_{E}R_{F})sulfonylalkyl;
R_{E} and R_{F} are each independently a member selected from the group consisting of hydrogen, alkoxyalkyl, alkoxyalkylcarbonyl, alkoxycarbonyl, alkoxysulfonyl, alkyl, alkylcarbonyl, alkylsulfonyl, alkylthioalkyl, alkylthioalkylcarbonyl, alkylthiocarbonyl, aryl, arylalkoxyalkyl, arylalkyl, arylcarbonyl, cycloalkyl, cycloalkylalkyl, cycloalkylcarbonyl, formyl, heteroaryl, heteroarylalkyl, heteroarylcarbonyl, heterocycle, heterocyclealkyl, heterocyclecarbonyl, (NZ₁Z₂)alkyl, and (NZ₁Z₂)carbonyl;
Z₁ and Z₂ are each independently a member selected from the group consisting of hydrogen, alkoxycarbonyl, alkyl, alkylcarbonyl, alkoxysulfonyl, alkylsulfonyl, aryl, arylalkyl, arylcarbonyl, cycloalkyl, cycloalkylalkyl, cycloalkylcarbonyl, formyl, heteroaryl, heteroarylalkyl, heteroarylcarbonyl, heterocycle, heterocyclealkyl, and heterocyclecarbonyl;
R₄ is a member selected from the group consisting of alkenyl, alkenyloxy, alkenyloxyalkyl, alkoxy, alkoxyalkoxy, alkoxyalkoxyalkyl, alkoxyalkyl, alkoxycarbonyl, alkoxycarbonylalkyl, alkoxysulfonyl, alkyl, alkylcarbonyl, alkylcarbonylalkyl, alkylcarbonyloxy, alkylsulfinyl, alkylsulfinylalkyl, alkylsulfonyl, alkylsulfonylalkyl, alkylthio, alkylthioalkyl, alkynyl, alkynyloxy, alkynyloxyalkyl, aryl, arylalkoxy, arylalkoxyalkyl, arylalkyl, arylalkylthio, arylalkylthioalkyl, aryloxy, aryloxyalkyl, arylthio, arylthioalkyl, carboxy, carboxyalkyl, cyano, cyanoalkyl, cycloalkenyl, cycloalkenylalkoxy, cycloalkenylalkoxyalkyl, cycloalkenylalkyl, cycloalkenylalkylthio, cycloalkenylalkylthioalkyl, cycloalkenyloxy, cycloalkenyloxyalkyl, cycloalkenylthio, cycloalkenylthioalkyl, cycloalkyl, cycloalkylalkoxy, cycloalkylalkoxyalkyl, cycloalkylalkyl, cycloalkylalkylthio, cycloalkylalkylthioalkyl, cycloalkyloxy, cycloalkyloxyalkyl, cycloalkylthio, cycloalkylthioalkyl, formyl, formylalkyl, haloalkoxy, haloalkyl, halogen, heteroaryl, heteroarylalkoxy, heteroarylalkoxyalkyl, heteroarylalkyl, heteroarylalkylthio, heteroarylalkylthioalkyl, heteroaryloxy, heteroaryloxyalkyl, heteroarylthio, heteroarylthioalkyl, heterocycle, heterocyclealkoxy, heterocyclealkoxyalkyl, heterocyclealkyl, heterocyclealkylthio, heterocyclealkylthioalkyl, heterocycleoxy, heterocycleoxyalkyl, heterocyclethio, heterocyclethioalkyl, hydroxy, hydroxyalkyl, mercapto, nitro, —NR_{G}R_{H}, (NR_{G}R_{H})alkyl, (NR_{G}R_{H})carbonyl, and (NR_{G}R_{H})sulfonyl;
R_{G} and R_{H} are each independently a member selected from the group consisting of hydrogen, alkoxyalkyl, alkoxyalkylcarbonyl, alkoxycarbonyl, alkoxysulfonyl, alkyl, alkylcarbonyl, alkylsulfonyl, alkylthioalkyl, alkylthioalkylcarbonyl, alkylthiocarbonyl, aryl, arylalkoxyalkyl, arylalkyl, arylcarbonyl, cycloalkyl, cycloalkylalkoxyalkyl, cycloalkylalkyl, cycloalkylcarbonyl, formyl, heteroaryl, heteroarylalkoxyalkyl, heteroarylalkyl, heteroarylcarbonyl, heterocycle, heterocyclealkoxyalkyl, heterocyclealkyl, heterocyclecarbonyl, (NZ₃Z₄)alkyl, and (NZ₃Z₄)carbonyl; and
Z₃ and Z₄ are each independently a member selected from the group consisting of hydrogen, alkoxycarbonyl, alkoxysulfonyl, alkyl, alkylcarbonyl, alkylsulfonyl, aryl, arylalkyl, arylcarbonyl, cycloalkyl, cycloalkylalkyl, cycloalkylcarbonyl, formyl, heteroaryl, heteroarylalkyl, heteroarylcarbonyl, heterocycle, heterocyclealkyl, and heterocyclecarbonyl.

4. A compound according to claim 3, that is a member selected from the group consisting of
6-[(Benzyloxy)methyl]-5-{4-[(4-chlorobenzyl)amino]phenyl}pyrimidine-2,4-diamine;
5-{4-[(4-Chlorobenzyl)amino]phenyl}-6-(methoxymethyl)pyrimidine-2,4-diamine;
5-{4-[(4-Chlorobenzyl)amino]phenyl}-6-{[(2-fluoro-3-methylbenzyl)oxy]methyl}pyrimidine-2,4-diamine;
5-{4-[(4-Chlorobenzyl)amino]phenyl}-6-(3-phenylpropyl)pyrimidine-2,4-diamine;
5-{4-[(4-Chlorobenzyl)amino]phenyl}-6-(phenoxymethyl)pyrimidine-2,4-diamine;
5-{4-[(4-Chlorobenzyl)amino]phenyl}-6-{[(3-methylbenzyl)oxy]methyl}pyrimidine-2,4-diamine;
5-{4-[(4-Chlorobenzyl)amino]phenyl}-6-{[(2-methoxybenzyl)oxy]methyl}pyrimidine-2,4-diamine;
5-{4-[(4-Chlorobenzyl)amino]phenyl}-6-{[(3-methoxybenzyl)oxy]methyl}pyrimidine-2,4-diamine;
5-{4-[(4-Chlorobenzyl)amino]phenyl}-6-{[(4-methoxybenzyl)oxy]methyl}pyrimidine-2,4-diamine;
5-{4-[(4-Chlorobenzyl)amino]phenyl}-6-{[(2-fluorobenzyl)oxy]methyl}pyrimidine-2,4-diamine;
5-{4-[(4-Chlorobenzyl)amino]phenyl}-6-{[(4-fluorobenzyl)oxy]methyl}pyrimidine-2,4-diamine;
5-{4-[(4-Chlorobenzyl)amino]phenyl}-6-{[(2-chlorobenzyl)oxy]methyl}pyrimidine-2,4-diamine;
5-{4-[(4-Chlorobenzyl)amino]phenyl}-6-{[(4-chlorobenzyl)oxy]methyl}pyrimidine-2,4-diamine;
6-{[(2-Bromobenzyl)oxy]methyl}-5-{4-[(4-chlorobenzyl)amino]phenyl}pyrimidine-2,4-diamine;
5-{4-[(4-Chlorobenzyl)amino]phenyl}-6-({[3-(trifluoromethyl)benzyl]oxy}methyl)pyrimidine-2,4-diamine;
5-{4-[(4-Chlorobenzyl)amino]phenyl}-6-( {[4-(methylthio)benzyl]oxy}methyl)pyrimidine-2,4-diamine;
5-{4-[(4-Chlorobenzyl)amino]phenyl}-6-{[(2,4-dimethylbenzyl)oxy]methyl}pyrimidine-2,4-diamine;
5-{4-[(4-Chlorobenzyl)amino]phenyl}-6-{[(3,5-dimethylbenzyl)oxy]methyl}pyrimidine-2,4-diamine;
5-{4-[(4-Chlorobenzyl)amino]phenyl}-6-{[(2,3-dichlorobenzyl)oxy]methyl}pyrimidine-2,4-diamine;
5-{4-[(4-Chlorobenzyl)amino]phenyl}-6-{[(2,5-dichlorobenzyl)oxy]methyl}pyrimidine-2,4-diamine;
5-{4-[(1,3-Benzodioxol-4-ylmethyl)amino]phenyl}-6-[(benzyloxy)methyl]pyrimidine-2,4-diamine;
tert-butyl 2-[(4-{2,4-Diamino-6-[(benzyloxy)methyl]pyrimidin-5-yl}phenyl)amino]ethylcarbamate;
6-[(Benzyloxy)methyl]-5-{4-[(3-furylmethyl)amino]phenyl}pyrimidine-2,4-diamine;
6-[(Benzyloxy)methyl]-5-{4-[(tetrahydrofuran-3-ylmethyl)amino]phenyl}pyrimidine-2,4-diamine;
4-Chloro-N-(4-{2,4-diamino-6-[(benzyloxy)methyl]pyrimidin-5-yl}phenyl)benzamide;
6-[(Benzyloxy)methyl]-5-{4-[(pyridin-2-ylmethyl)amino]phenyl}pyrimidine-2,4-diamine;
6-[(Benzyloxy)methyl]-5-{4-[(pyridin-3-ylmethyl)amino]phenyl}pyrimidine-2,4-diamine;
6-[(Benzyloxy)methyl]-5-{4-[(1H-imidazol-4-ylmethyl)amino]phenyl}pyrimidine-2,4-diamine;
6-[(Benzyloxy)methyl]-5-[4-(dimethylamino)phenyl]pyrimidine-2,4-diamine;
6-[(Benzyloxy)methyl]-5-[4-(methylamino)phenyl]pyrimidine-2,4-diamine;
6-[(Benzyloxy)methyl]-5-[4-(ethylamino)phenyl]pyrimidine-2,4-diamine;
6-[(Benzyloxy)methyl]-5-[4-(propylamino)phenyl]pyrimidine-2,4-diamine;
6-[(Benzyloxy)methyl]-5-[4-(isobutylamino)phenyl]pyrimidine-2,4-diamine;
6-[(Benzyloxy)methyl]-5-[4-(neopentylamino)phenyl]pyrimidine-2,4-diamine;
6-[(Benzyloxy)methyl]-5-{4-[(cyclopropylmethyl)amino]phenyl}pyrimidine-2,4-diamine;
2-Butoxy-N-(4-{2,4-diamino-6-[(benzyloxy)methyl]pyrimidin-5-yl}phenyl)acetamide;
5-{4-[(4-Chlorobenzyl)amino]phenyl}-6-tetrahydrofuran-2-ylpyrimidine-2,4-diamine;
6-[(2-Butoxyethoxy)methyl]-5-{4-[(4-chlorobenzyl)amino]phenyl}pyrimidine-2,4-diamine;
6-[(Benzyloxy)methyl]-5-{4-[(1-ethylpropyl)amino]phenyl}pyrimidine-2,4-diamine;
4-{[(4-{2,4-Diamino-6-[(benzyloxy)methyl]pyrimidin-5-yl}phenyl)amino]methyl}benzonitrile;
4-{[(4-{2,4-Diamino-6-[(benzyloxy)methyl]pyrimidin-5-yl}phenyl)(methyl)amino]methyl}benzonitrile;
5-{4-[(4-Chlorobenzyl)amino]phenyl}-6-[(3-methylbutoxy)methyl]pyrimidine-2,4-diamine;
N-(4-{2,4-Diamino-6-[(benzyloxy)methyl]pyrimidin-5-yl}phenyl)propanamide;
6-[(Benzyloxy)methyl]-5-{4-[(pyridin-4-ylmethyl)amino]phenyl}pyrimidine-2,4-diamine;
N-(4-Chlorobenzyl)-N-(4-{2,4-diamino-6-[(benzyloxy)methyl]pyrimidin-5-yl}phenyl)acetamide;
4-Chlorobenzyl(4-{2,4-diamino-6-[(benzyloxy)methyl]pyrimidin-5-yl}phenyl)formamide;
6-[(Benzyloxy)methyl]-5-{4-[(1H-imidazol-2-ylmethyl)amino]phenyl}pyrimidine-2,4-diamine;
6-[(Benzyloxy)methyl]-5-(4-{[(6-chloropyridin-3-yl)methyl]amino}phenyl)pyrimidine-2,4-diamine;
N-benzyl-3-(2,6-diamino-5-{4-[(4-chlorobenzyl)amino]phenyl}pyrimidin-4-yl)propanamide;
3-(2,6-Diamino-5-{4-[(4-chlorobenzyl)amino]phenyl}pyrimidin-4-yl)-N-phenylpropanamide;
6-[(Benzyloxy)methyl]-5-{4-[( 1-pyridin-4-ylethyl)amino]phenyl }pyrimidine-2,4-diamine;
4-{1-[(4-{2,4-Diamino-6-[(benzyloxy)methyl]pyrimidin-5-yl}phenyl)amino]ethyl}benzonitrile;
6-[(Benzyloxy)methyl]-5-{4-[(4-methoxybenzyl)amino]phenyl}pyrimidine-2,4-diamine;
6-[(Benzyloxy)methyl]-5-(4-{[1-(4-chlorophenyl)ethyl]amino}phenyl)pyrimidine-2,4-diamine;
6-[(Benzyloxy)methyl]-5-{4-[(cyclohexylmethyl)amino]phenyl}pyrimidine-2,4-diamine;
N-butyl-3-(2,6-diamino-5-{4-[(4-chlorobenzyl)amino]phenyl}pyrimidin-4-yl)propanamide;
3-(2,6-Diamino-5-{4-[(4-chlorobenzyl)amino]phenyl}pyrimidin-4-yl)-N-(3-methylphenyl)propanamide;
6-[(Benzyloxy)methyl]-5-{4-[(4-chlorobenzyl)oxy]phenyl}pyrimidine-2,4-diamine;
6-[(Benzyloxy)methyl]-5-(4-{[(4-chlorobenzyl)amino]methyl}phenyl)pyrimidine-2,4-diamine;
5-[4-(Benzylamino)phenyl]-6-[(benzyloxy)methyl]pyrimidine-2,4-diamine;
6-[(Benzyloxy)methyl]-5-(4-{[(4-nitrophenyl)amino]methyl}phenyl)pyrimidine-2,4-diamine;
N-(4-{2,4-Diamino-6-[(benzyloxy)methyl]pyrimidin-5-yl}benzyl)-N′-propylurea;
4-{[(4-{2,4-Diamino-6-[(cyclobutylmethoxy)methyl]pyrimidin-5-yl}phenyl)amino]methyl}benzonitrile;
4-[(4-{2,4-Diamino-6-[(benzyloxy)methyl]pyrimidin-5-yl}phenoxy)methyl]benzonitrile;
5-{4-[(4-Chlorobenzyl)amino]phenyl}-6-[(tetrahydro-2H-pyran-2-ylmethoxy)methyl]pyrimidine-2,4-diamine;
6-[(Benzyloxy)methyl]-5-[4-({[6-(trifluoromethyl)pyridin-3-yl]methyl}amino)phenyl]pyrimidine-2,4-diamine;
4-[(4-{2,4-Diamino-6-[(benzyloxy)methyl]pyrimidin-5-yl}benzyl)amino]benzonitrile;
3-[(4-{2,4-Diamino-6-[(benzyloxy)methyl]pyrimidin-5-yl}phenoxy)methyl]benzonitrile;
5-{[(4-{2,4-Diamino-6-[(benzyloxy)methyl]pyrimidin-5-yl}phenyl)amino]methyl}pyridine-2-carbonitrile;
6-[(Benzyloxy)methyl]-5-{4-[2-(4-chlorophenyl)ethoxy]phenyl}pyrimidine-2,4-diamine;
6-[(Benzyloxy)methyl]-5-[4-(pyridin-3-ylmethoxy)phenyl]pyrimidine-2,4-diamine;
6-[(Benzyloxy)methyl]-5-{4-[(tetrahydro-2H-pyran-4-ylmethyl)amino]phenyl}pyrimidine-2,4-diamine;
6-[(Benzyloxy)methyl]-5-(4-{[4-(trifluoromethoxy)benzyl]amino}phenyl)pyrimidine-2,4-diamine;
5-{4-[(4-Chlorobenzyl)amino]phenyl}-6-[(cyclohexylmethoxy)methyl]pyrimidine-2,4-diamine;
4-{[(4-{2,4-Diamino-6-[(benzyloxy)methyl]pyrimidin-5-yl}phenyl)amino]methyl}pyridine-2-carbonitrile;
6-[(4-{2,4-Diamino-6-[(benzyloxy)methyl]pyrimidin-5-yl}benzyl)amino]nicotinonitrile;
5-{4-[(4-Chlorobenzyl)amino]phenyl}-6-{[(3-chlorobenzyl)oxy]methyl}pyrimidine-2,4-diamine;
5-{4-[(4-Chlorobenzyl)amino]phenyl}-6-{[(2-methylbenzyl)oxy]methyl}pyrimidine-2,4-diamine;
6-[(Benzyloxy)methyl]-5-{4-[(4-nitrobenzyl)amino]phenyl}pyrimidine-2,4-diamine;
6-[(Benzyloxy)methyl]-5-(4-{[(2-chloropyridin-4-yl)methyl]amino}phenyl)pyrimidine-2,4-diamine;
6-[(Benzyloxy)methyl]-5-{4-[(pyrimidin-5-ylmethyl)amino]phenyl}pyrimidine-2,4-diamine;
6-[(Benzyloxy)methyl]-5-{4-[(thien-2-ylmethyl)amino]phenyl}pyrimidine-2,4-diamine;
6-[(Benzyloxy)methyl]-5-{4-[(thien-3-ylmethyl)amino]phenyl}pyrimidine-2,4-diamine;
6-[(Benzyloxy)methyl]-5-[4-({[1-(4-chlorophenyl)ethyl]amino}methyl)phenyl]pyrimidine-2,4-diamine;
6-[(Benzyloxy)methyl]-5-(4-{[2-(4-nitrophenyl)ethyl]amino}phenyl)pyrimidine-2,4-diamine;
6-[(Benzyloxy)methyl]-5-(4-{[2-(4-chlorophenyl)ethyl]amino}phenyl)pyrimidine-2,4-diamine;
6-[(Benzyloxy)methyl]-5-{4-[(cycloheptylamino)methyl]phenyl}pyrimidine-2,4-diamine; and
6-Benzyloxymethyl-5-[4-(pyridin-4-ylmethoxy)-phenyl]-pyrimidine-2,4-diamine.

5. A compound according to claim 3, that is a member selected from the group consisting of
5-{4-[(4-Chlorobenzyl)amino]phenyl}-6-ethylpyrimidine-2,4-diamine;
4-({[4-(2,4-Diamino-6-ethylpyrimidin-5-yl)phenyl]amino}methyl)benzonitrile;
5-{4-[(3,4-Dichlorobenzyl)amino]phenyl}-6-ethylpyrimidine-2,4-diamine;
5-{4-[(4-Chlorobenzyl)amino]phenyl}-6-propylpyrimidine-2,4-diamine;
5-(4-{[2-(Benzyloxy)ethyl]amino}phenyl)-6-ethylpyrimidine-2,4-diamine; and
6-Ethyl-5-{4-[(4-nitrobenzyl)amino]phenyl}pyrimidine-2,4-diamine.

6. A pharmaceutical composition comprising a therapeutically effective amount of a compound of formula (I) and a pharmaceutically suitable carrier.

7. A method of treating a disorder regulated by ghrelin receptors in a mammal, comprising administring of a compound of formula (I).

8. The method according to claim 7 wherein the disorder is a member selected from the group consisting of anorexia, cancer cachexia, eating disorders, age-related decline in body composition, weight gain, obesity, and diabetes mellitus.

9. A method of treating a disorder regulated by ghrelin receptors in a mammal comprising administering to the mammal a therapeutically effective amount of a compound of formula (II) 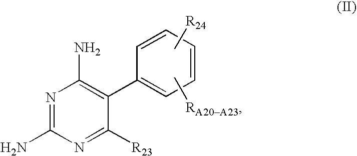
or a therapeutically suitable salt or prodrug thereof, wherein
R₂₃ is a member selected from the group consisting of hydrogen, alkyl, haloalkyl, cyano, and (NR₂₅R₂₆)carbonyl;
R₂₄ is a member selected from the group consisting of alkenyl, alkenyloxy, alkoxy, alkoxyalkoxy, alkoxyalkoxyalkyl, alkoxyalkyl, alkoxycarbonyl, alkoxycarbonylalkyl, alkoxysulfonyl, alkyl, alkylcarbonyl, alkylcarbonylalkyl, alkylcarbonyloxy, alkylsulfinyl, alkylsulfinylalkyl, alkylsulfonyl, alkylsulfonylalkyl, alkylthio, alkylthioalkyl, alkynyl, aryl, arylalkoxy, arylalkoxyalkyl, arylalkyl, arylalkylthio, arylalkylthioalkyl, aryloxy, aryloxyalkyl, arylthio, arylthioalkyl, carboxy, carboxyalkyl, cyano, cyanoalkyl, cycloalkenyl, cycloalkenylalkoxy, cycloalkenylalkoxyalkyl, cycloalkenylalkyl, cycloalkenylalkylthio, cycloalkenylalkylthioalkyl, cycloalkenyloxy, cycloalkenyloxyalkyl, cycloalkenylthio, cycloalkenylthioalkyl, cycloalkyl, cycloalkylalkoxy, cycloalkylalkoxyalkyl, cycloalkylalkyl, cycloalkylalkylthio, cycloalkylalkylthioalkyl, cycloalkyloxy, cycloalkyloxyalkyl, cycloalkylthio, cycloalkylthioalkyl, formyl, formylalkyl, haloalkoxy, haloalkyl, halogen, heteroaryl, heteroarylalkoxy, heteroarylalkoxyalkyl, heteroarylalkyl, heteroarylalkylthio, heteroarylalkylthioalkyl, heteroaryloxy, heteroaryloxyalkyl, heteroarylthio, heteroarylthioalkyl, heterocycle, heterocyclealkoxy, heterocyclealkoxyalkyl, heterocyclealkyl, heterocyclealkylthio, heterocyclealkylthioalkyl, heterocycleoxy, heterocycleoxyalkyl, heterocyclethio, heterocyclethioalkyl, hydroxy, hydroxyalkyl, mercapto, nitro, —NR₂₇R₂₈, (NR₂₇R₂₈)alkyl, (NR₂₇R₂₈)carbonyl, and (NR₂₇R₂₈)sulfonyl;
R₂₅ and R₂₆ are each independently a member selected from the group consisting of hydrogen, alkyl, and alkylcarbonyl;
R₂₇ and R₂₈ are each independently a member selected from the group consisting of hydrogen, alkoxyalkyl, alkoxyalkylcarbonyl, alkoxycarbonyl, alkoxysulfonyl, alkyl, alkylcarbonyl, alkylsulfonyl, alkylthioalkyl, alkylthioalkylcarbonyl, alkylthiocarbonyl, aryl, arylalkoxyalkyl, arylalkyl, arylcarbonyl, cycloalkyl, cycloalkylalkoxyalkyl, cycloalkylalkyl, cycloalkylcarbonyl, formyl, heteroaryl, heteroarylalkoxyalkyl, heteroarylalkyl, heteroarylcarbonyl, heterocycle, heterocyclealkoxyalkyl, heterocyclealkyl, heterocyclecarbonyl, (NZ₂₃Z₂₄)alkyl, and (NZ₂₃Z₂₄)carbonyl;
Z₂₃ and Z₂₄ are each independently a member selected from the group consisting of hydrogen, alkoxycarbonyl, alkoxysulfonyl, alkyl, alkylcarbonyl, alkylsulfonyl, aryl, arylalkyl, arylcarbonyl, cycloalkyl, cycloalkylalkyl, cycloalkylcarbonyl, formyl, heteroaryl, heteroarylalkyl, heteroarylcarbonyl, heterocycle, heterocyclealkyl, and heterocyclecarbonyl;
R_{A20}, R_{A21}, R_{A22}, and R_{A23} are each independently a member selected from the group consisting of hydrogen, alkenyl, alkenyloxy, alkoxy, alkoxyalkoxy, alkoxyalkoxyalkyl, alkoxyalkyl, alkoxycarbonyl, alkoxycarbonylalkyl, alkoxysulfonyl, alkyl, alkylcarbonyl, alkylcarbonylalkyl, alkylcarbonyloxy, alkylsulfinyl, alkylsulfinylalkyl, alkylsulfonyl, alkylsulfonylalkyl, alkylthio, alkylthioalkyl, alkynyl, carboxy, carboxyalkyl, cyano, cyanoalkyl, formyl, haloalkoxy, haloalkyl, halogen, hydroxy, hydroxyalkyl, mercapto, nitro, —NR₃₀R₃₁, (NR₃₀R₃₁)alkyl, (NR₃₀R₃₁)carbonyl, and (NR₃₀R₃₁)sulfonyl; and
R₃₀ and R₃₁ are each independently a member selected from the group consisting of hydrogen, alkoxycarbonyl, alkyl, alkylcarbonyl, alkoxysulfonyl, alkylsulfonyl, aryl, arylalkyl, and formyl.

10. A method of treating a disorder regulated by ghrelin receptors in a mammal comprising administering to the mammal a therapeutically effective amount of a compound of formula (IIa) 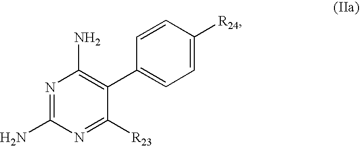
or a therapeutically suitable salt or prodrug thereof, wherein
R₂₃ is a member selected from the group consisting of hydrogen, alkyl, haloalkyl, cyano, and (NR₂₅R₂₆)carbonyl;
R₂₄ is a member selected from the group consisting of alkenyl, alkenyloxy, alkenyloxyalkyl, alkoxy, alkoxyalkoxy, alkoxyalkoxyalkyl, alkoxyalkyl, alkoxycarbonyl, alkoxycarbonylalkyl, alkoxysulfonyl, alkyl, alkylcarbonyl, alkylcarbonylalkyl, alkylcarbonyloxy, alkylsulfinyl, alkylsulfinylalkyl, alkylsulfonyl, alkylsulfonylalkyl, alkylthio, alkylthioalkyl, alkynyl, alkynyloxy, alkynyloxyalkyl, aryl, arylalkoxy, arylalkoxyalkyl, arylalkyl, arylalkylthio, arylalkylthioalkyl, aryloxy, aryloxyalkyl, arylthio, arylthioalkyl, carboxy, carboxyalkyl, cyano, cyanoalkyl, cycloalkenyl, cycloalkenylalkoxy, cycloalkenylalkoxyalkyl, cycloalkenylalkyl, cycloalkenylalkylthio, cycloalkenylalkylthioalkyl, cycloalkenyloxy, cycloalkenyloxyalkyl, cycloalkenylthio, cycloalkenylthioalkyl, cycloalkyl, cycloalkylalkoxy, cycloalkylalkoxyalkyl, cycloalkylalkyl, cycloalkylalkylthio, cycloalkylalkylthioalkyl, cycloalkyloxy, cycloalkyloxyalkyl, cycloalkylthio, cycloalkylthioalkyl, formyl, formylalkyl, haloalkoxy, haloalkyl, halogen, heteroaryl, heteroarylalkoxy, heteroarylalkoxyalkyl, heteroarylalkyl, heteroarylalkylthio, heteroarylalkylthioalkyl, heteroaryloxy, heteroaryloxyalkyl, heteroarylthio, heteroarylthioalkyl, heterocycle, heterocyclealkoxy, heterocyclealkoxyalkyl, heterocyclealkyl, heterocyclealkylthio, heterocyclealkylthioalkyl, heterocycleoxy, heterocycleoxyalkyl, heterocyclethio, heterocyclethioalkyl, hydroxy, hydroxyalkyl, mercapto, nitro, —NR₂₇R₂₈, (NR₂₇R₂₈)alkyl, (NR₂₇R₂₈)carbonyl, and (NR₂₇R₂₈)sulfonyl;
R₂₅ and R₂₆ are each independently a member selected from the group consisting of hydrogen, alkyl, and alkylcarbonyl;
R₂₇ and R₂₈ are each independently a member selected from the group consisting of hydrogen, alkoxyalkyl, alkoxyalkylcarbonyl, alkoxycarbonyl, alkoxysulfonyl, alkyl, alkylcarbonyl, alkylsulfonyl, alkylthioalkyl, alkylthioalkylcarbonyl, alkylthiocarbonyl, aryl, arylalkoxyalkyl, arylalkyl, arylcarbonyl, cycloalkyl, cycloalkylalkoxyalkyl, cycloalkylalkyl, cycloalkylcarbonyl, formyl, heteroaryl, heteroarylalkoxyalkyl, heteroarylalkyl, heteroarylcarbonyl, heterocycle, heterocyclealkoxyalkyl, heterocyclealkyl, heterocyclecarbonyl, (NZ₂₃Z₂₄)alkyl, and (NZ₂₃Z₂₄)carbonyl; and
Z₂₃ and Z₂₄ are each independently a member selected from the group consisting of hydrogen, alkoxycarbonyl, alkoxysulfonyl, alkyl, alkylcarbonyl, alkylsulfonyl, aryl, arylalkyl, arylcarbonyl, cycloalkyl, cycloalkylalkyl, cycloalkylcarbonyl, formyl, heteroaryl, heteroarylalkyl, heteroarylcarbonyl, heterocycle, heterocyclealkyl, and heterocyclecarbonyl.

11. A pharmaceutical composition comprising a therapeutically effective amount of a compound of formula (II) and a pharmaceutically suitable carrier.

12. The method according to claim 9 wherein the disorder is a member selected from the group consisting of anorexia, cancer cachexia, eating disorders, age-related decline in body composition, weight gain, obesity, and diabetes mellitus.
